SEQUENCE LISTING (35 sequences)

SEQ ID NO: 1 - DNA, 51 bp, Homo Sapiens
  variation (26)..(26): StatinSNP1_C26G
acttacggccaccttgctcctccgcscttcacctcatcgccccctctttct

SEQ ID NO: 2 - DNA, 51 bp, Homo Sapiens
  variation (26)..(26): StatinSNP2_A26G
accggaaacaaatggctgtcaagaartacttggcggccgtcctagggaaga

SEQ ID NO: 3 - DNA, 51 bp, Homo Sapiens
  variation (26)..(26): StatinSNP3_G26A
tggaatatctgggtttcgttaacagrttcaaattgttcaaaccatggactt

SEQ ID NO: 4 - DNA, 51 bp, Homo Sapiens
  variation (26)..(26): StatinSNP4_A26G
gtcttgcagcaagtgggaggacgaarcaggtttcataagagcagcgcccaa

SEQ ID NO: 5 - DNA, 51 bp, Homo Sapiens
  variation (26)..(26): StatinSNP5_C26T
gggccgccagccacagcctgtgcacytgctgcccctgcacgggatacagca

SEQ ID NO: 6 - DNA, 51 bp, Homo Sapiens
  variation (26)..(26): StatinSNP6_C26T
ccgcacccggccccaaacgtttaaayagatgtatttcagaactttcagata

SEQ ID NO: 7 - DNA, 51 bp, Homo Sapiens
  variation (26)..(26): StatinSNP7_A26T
tgatctttactcatttaatgaaattwctgattgtctcatatctattgtctt

SEQ ID NO: 8 - DNA, 51 bp, Homo Sapiens
  variation (26)..(26): StatinSNP8_T26G
ggtgccaccctgggtttgagagtgtktgtttgtttagctttttggtgattt

SEQ ID NO: 9 - DNA, 51 bp, Homo Sapiens
  variation (26)..(26): StatinSNP9_G26A
aaataattaccacctttgatttcctrttcaaaattttcagcctccaatctt

SEQ ID NO: 10 - DNA, 51 bp, Homo Sapiens
  variation (26)..(26): StatinSNP10_G26A
tacacgaagttacattagggagaaartaaaagaacaccaagcatcactgga

SEQ ID NO: 11 - DNA, 51 bp, Homo Sapiens
  variation (26)..(26): StatinSNP11_C26T
gaagaatgctagcccatctggctgcygatctgctatcacctgcaactcttt

SEQ ID NO: 12 - DNA, 51 bp, Homo Sapiens
  variation (26)..(26): StatinSNP12_T26C
cagcctggctggggccacgggtgttyggggccgggtgtcgcggccgcgccc

SEQ ID NO: 13 - DNA, 51 bp, Homo Sapiens
  variation (26)..(26): StatinSNP13_G26C
atggagttgaagacccagtcctgatsgccctgtagcctgtctgacctgtgg

SEQ ID NO: 14 - DNA, 51 bp, Homo Sapiens
  variation (26)..(26): StatinSNP14_C26G
gcagctgtgtgccgtgaggtaaggcsaagggccaagcagtcttgggtagaa

SEQ ID NO: 15 - DNA, 51 bp, Homo Sapiens
  variation (26)..(26): StatinSNP15_C26T
cagcaccctttacaaacaaagatgcytttatgtcttgtaatggttactggg

SEQ ID NO: 16 - DNA, 51 bp, Homo Sapiens
  variation (26)..(26): StatinSNP16_G26C
gaatggtgggggccataacctgggtsggaacttgtaacagtctcccacatc

SEQ ID NO: 17 - DNA, 51 bp, Homo Sapiens
  variation (26)..(26): StatinSNP17_G26A
ccgtggagccacatggcgagatgaartttcagtgggacctcaatgcctgga

SEQ ID NO: 18 - DNA, 51 bp, Homo Sapiens
  variation (26)..(26): StatinSNP18_A26T
gccgggtagatgcatatatatatatwtttttctaactatagcaagcaagaa

SEQ ID NO: 19 - DNA, 51 bp, Homo Sapiens
  variation (26)..(26): StatinSNP19_C26T
cggggccaggcctgcggagtgctgaygcagagtgactgggtgtccggcagc

SEQ ID NO: 20 - DNA, 51 bp, Homo Sapiens
  variation (26)..(26): StatinSNP20_C26T
gctacggcgaggaggggcgcgattgytccttgttgccgctccgcttagtgg

SEQ ID NO: 21 - DNA, 51 bp, Homo Sapiens
  variation (26)..(26): StatinSNP21_A26G
tccggctctctggtccactcaaggarcagtatgctcaggtaggtggtgctt

SEQ ID NO: 22 - DNA, 51 bp, Homo Sapiens
  variation (26)..(26): StatinSNP22_G26A
cataaaaccctaatgtcttcctctgrgtaacaacacagagggagaaggtgg

SEQ ID NO: 23 - DNA, 51 bp, Homo Sapiens
  variation (26)..(26): StatinSNP23_A26G
tcttcctctgggtaacaacacagagrgagaaggtggggacaggtgcaggga

SEQ ID NO: 24 - DNA, 51 bp, Homo Sapiens
  variation (26)..(26): StatinSNP24_T26G
aagtatttcagggaatcatttaaatkatcattttaggtttctttgtttgat

SEQ ID NO: 25 - DNA, 51 bp, Homo Sapiens
  variation (26)..(26): StatinSNP25_A26G
aatattgtaaattggaaggtatgcartgttggttaaccactgtgacctcat

SEQ ID NO: 26 - DNA, 51 bp, Homo Sapiens
  variation (26)..(26): StatinSNP26_A26G
tcttggcccctacggacctcagaggragcatgagcacgtgtcacccgtcct

SEQ ID NO: 27 - DNA, 51 bp, Homo Sapiens
  variation (26)..(26): StatinSNP27_A26T
ctcatatcgctggtcttgacacagcwttttatttgtaaggataaaaaatag

SEQ ID NO: 28 - DNA, 51 bp, Homo Sapiens
  variation (26)..(26): StatinSNP28_C26T
cccgtgccccccttactatttagtgytgagattgattttttctctctcttt

SEQ ID NO: 29 - DNA, 51 bp, Homo Sapiens
  variation (26)..(26): StatinSNP29_C26T
gaaagtttcctcggggcttcaaattygacacggatgagctgaactttccca

SEQ ID NO: 30 - DNA, 51 bp, Homo Sapiens
  variation (26)..(26): StatinSNP30_C26T
aacctctgatgctgctgacactctcytccattgctttcagagtcacctggt

SEQ ID NO: 31 - DNA, 51 bp, Homo Sapiens
  variation (26)..(26): StatinSNP31_G26A
tgacgtgatggtgagctcccccttgrtgcccagctccagcgattcagccca

SEQ ID NO: 32 - DNA, 51 bp, Homo Sapiens
  variation (26)..(26): StatinSNP32_C26T
gaagcctcactcccttctctcctggygcagacacgtccccatcagaaggca

SEQ ID NO: 33 - DNA, 51 bp, Homo Sapiens
  variation (26)..(26): StatinSNP33_C26T
cagaatgagcgggaccggatcagcaytcgaaggtcaagctatgaggacagc

SEQ ID NO: 34 - DNA, 51 bp, Homo Sapiens
  variation (26)..(26): StatinSNP34_C26T
ggagttagggcttcactggccacatycacagggtttggccttcataatcta

SEQ ID NO: 35 - DNA, 51 bp, Homo Sapiens
  variation (26)..(26): StatinSNP35_G26A
atgtagatggccttggtttaggggtrttgccaaggtgggatgaggttgatg

(19) United States
(12) Patent Application Publication
(10) Pub. No.: US 2009/0208945 A1
(43) Pub. Date: Aug. 20, 2009

(54) Method for the Prediction of Adverse Drug Responses to Stains

(75) Inventors: Stephan Schwers, Koln (DE); Udo Stropp, Haan (DE); Harald Kallabis, Leverkusen (DE); Andreas Schuppert, Kurten (DE); Rolf Burghaus, Kaarst (DE); Christian Von Torne, Solingen (DE); Gerd Schmitz, Sinzing (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS DIAGNOSTICS GMBH, Erlangen (DE)

(21) Appl. No.: 12/097,961

(22) PCT Filed: Dec. 19, 2006
(86) PCT No.: PCT/EP2006/012265

§ 371 Date: Jun. 18, 2008

(30) Foreign Application Priority Data
Dec. 22, 2005 (EP) 05028157.5

(51) Int. Cl. C12Q 1/68

(52) U.S. Cl. 435/6

(57) ABSTRACT

The invention provides diagnostic methods and kits including oligo and/or polynucleotides or derivatives, including as well antibodies determining whether a human subject is at risk of getting adverse drug reaction after statin therapy. Still further the invention provides polymorphic sequences and other genes. The present invention further relates to isolated polynucleotides encoding a SADR gene polypeptide useful in methods to identify therapeutic agents and useful for preparation of a medicament to treat statin induced adverse drug reactions (SADR), the polynucleotide is selected from the group comprising: SEQ ID 1-35 with allelic variation as indicated in the sequences section contained in a functional surrounding like full length cDNA for SADR gene polypeptide and with or without the SADR gene promoter sequence.

### TECHNICAL FIELD

This invention relates to genetic polymorphisms useful for assessing the response to lipid lowering drug therapy and adverse drug reactions of those medicaments. In addition it relates to genetic polymorphisms useful for assessing risks in response to medications relevant to cardiovascular disease. Further, the present invention provides methods for the identification and therapeutic use of compounds as treatments of cardiovascular disease or as prophylactic therapy for cardiovascular diseases. Moreover, the present invention provides methods for the diagnostic monitoring of patients undergoing clinical evaluation for the treatment of cardiovascular disease, and for monitoring the efficacy of compounds in clinical trials. Still further, the present invention provides methods to use gene variations to predict personal medication schemes omitting adverse drug reactions and allowing an adjustment of the drug dose to achieve maximum benefit for the patient.

### BACKGROUND OF THE INVENTION

Cardiovascular disease (CVD) is a major health risk throughout the industrialized world. It is estimated that nearly 40% of all deaths annually are caused by CVD.

Cardiovascular diseases include but are not limited to the following disorders of the heart and the vascular system: congestive heart failure, myocardial infarction, atherosclerosis, ischemic diseases of the heart, coronary heart disease, all kinds of atrial and ventricular arrhythmias, hypertensive vascular diseases and peripheral vascular diseases.

At present, the only available treatments for cardiovascular disorders are pharmaceutical based medications that are not targeted to an individual's actual defect; examples include angiotensin converting enzyme (ACE) inhibitors and diuretics for hypertension, insulin supplementation for non-insulin dependent diabetes mellitus (NIDDM), cholesterol reduction strategies for dyslipidaemia (see below), anticoagulants, β blockers for cardiovascular disorders and weight reduction strategies for obesity.

### Dyslipidaemia Treatment and Adverse Drug Reactions

Adverse drug reactions (ADRs) remain a major clinical problem. A recent meta-analysis suggested that in the USA in 1994, ADRs were responsible for 100 000 deaths, making them between the fourth and sixth commonest cause of death (Lazarou 1998, J. Am. Med. Assoc. 279:1200). Although these figures have been heavily criticized, they emphasize the importance of ADRs. Indeed, there is good evidence that ADRs account for 5% of all hospital admissions and increase the length of stay in hospital by two days at an increased cost of ˜$2500 per patient. ADRs are also one of the commonest causes of drug withdrawal, which has enormous financial implications for the pharmaceutical industry. ADRs, perhaps fortunately, only affect a minority of those taking a particular drug. Although factors that determine susceptibility are unclear in most cases, there is increasing interest in the role of genetic factors. Indeed, the role of inheritable variations in predisposing patients to ADRs has been appreciated since the late 1950s and early 1960s through the discovery of deficiencies in enzymes such as pseudocholinesterase (butyrylcholinesterase) and glucose-6-phosphate dehydrogenase (G6PD). More recently, with the first draft of the human genome just completed, there has been renewed interest in this area with the introduction of terms such as pharmacogenomics and toxicogenomics. Essentially, the aim of pharmacogenomics and pharmacogenetics is to produce personalized medicines, whereby administration of the drug class and dosage is tailored to an individual genotype. Thus, the term pharmacogenetics embraces both efficacy and toxicity.

The 3-hydroxy-3-methylglutaryl coenzyme A (HMG-CoA) reductase inhibitors (“statins”) specifically inhibit the enzyme HMG-CoA reductase which catalyzes the rate limiting step in cholesterol biosynthesis. These drugs are effective in reducing the primary and secondary risk of coronary artery disease and coronary events, such as heart attack, in middle-aged and older men and women, in both diabetic and non-diabetic patients, and are often prescribed for patients with hyperlipidemia. Statins used in secondary prevention of coronary artery or heart disease significantly reduce the risk of stroke, total mortality and morbidity and attacks of myocardial ischemia; the use of statins is also associated with improvements in endothelial and fibrinolytic functions and decreased platelet thrombus formation.

Statins are the most widely prescribed drugs worldwide with annual growth rates of 15%. In addition to their proven efficacy regarding treatment of CVD, statins may be effective in indications as different as multiple sclerosis, dementia, osteoporosis and cancer. Those pleiotropic statin effects will probably lead to an even more widespread use of this drug class in the future.

The tolerability of statins during long term administration is an important issue. Adverse reactions involving skeletal muscle are not uncommon, and sometimes serious adverse reactions involving skeletal muscle such as myopathy and rhabdomyolysis may occur, requiring discontinuation of the drug. In addition an increase in serum creatine kinase (CK) may be a sign of a statin related adverse event. The dimension of such adverse events can be read from the extend of the CK level increase (as compared to the upper limit of normal [ULN]).

Occasionally arthralgia, alone or in association with myalgia, has been reported. Also an elevation of liver transaminases has been associated with statin administration.

It was shown that the drug response to statin therapy is a class effects, i.e. all known and presumably also all so far undiscovered statins share the same beneficial and harmful effects (Ucar, M. et al., Drug Safety 2000, 22:441). It follows that the discovery of diagnostic tools to predict the drug response to a single statin will also be of aid to guide therapy with other statins.

The present invention provides diagnostic tests to predict a patient's individual response to statin therapy. Such responses include, but are not limited to the extent of adverse drug reactions, the level of lipid lowering or the drug's influence on disease states. Those diagnostic tests may predict the response to statin therapy either alone or in combination with another diagnostic test or another drug regimen.

The invention may also be of use in confirming or corroborating the results of other diagnostic methods. The diagnosis of the invention may thus suitably be used either as an isolated technique or in combination with other methods and apparatus for diagnosis, in which latter case the invention provides a further test on which a diagnosis may be assessed.

Furthermore the present invention discloses genes that were found to be associated with statin ADR. Those genes, their gene products and the metabolic pathways in which they are involved are potential new therapeutic targets to treat statin induced ADR (SADR). In addition they might lead to new treatments for dyslipidaemia which are not prone to ADR. Furthermore they might lead to new treatments for all other indications in which drugs of the statin class are beneficial, including but not limited to multiple sclerosis, dementia, osteoporosis and cancer.

The present invention stems from using allelic association as a method for genotyping individuals; allowing the investigation of the molecular genetic basis for response to statin drugs. In a specific embodiment the invention tests for the polymorphisms in the sequences of the listed genes in the Examples. The invention demonstrates a link between this polymorphisms and predispositions to statin induced ADR by showing that allele frequencies significantly differ when individuals with good statin tolerability are compared to individuals exhibiting ADR under statin treatment (statin induced ADR, SADR). The meaning of “good statin tolerability” and “SADR” is defined in Table 1a.

Certain disease states would benefit, that is to say the suffering of the patient may be reduced or prevented or delayed, by administration of treatment or therapy in advance of disease appearance; this can be more reliably carried out if advance diagnosis of predisposition or susceptibility to disease can be diagnosed. Regarding dyslipidemia a number of different treatments exist, including but not limited to statins, bile acid binding resins (e.g. cholestyramine, colesevelam and colestipol), fibrates (e.g. clofibrate and gemfibrozil), nicotinic acids and others (e.g. ezetimibe). Hence if a diagnostic test as disclosed in the current invention would indicate a patient's predisposition to statin ADR, physicians could immediately start treatment with an alternative drug class.

### BRIEF DESCRIPTION OF THE INVENTION

The invention relates to a
1. Method for predicting drug response in a patient comprising the steps of
   (i) classification of said patient to one of several classes of patients using clinical parameters of said patient,
   (ii) predicting drug response of said patient from class specific genomic markers.
2. Method of count 1, wherein the drug response is an adverse drug reaction.
3. Method of count 1-2, wherein said genomic markers are a set of SNPs.
4. Method of counts 1-3 wherein said drug response is adverse drug reaction in statin therapy.
5. Method of count 14, wherein said clinical parameters are selected from the group consisting of
   (i) gender
   (ii) creatine kinase serum activity
   (iii) LDL serum level
   (iv) HDL serum level
   (v) cholesterol serum level
   (vi) alkaline phosphatase serum activity.
6. Method of counts 1-5, wherein said drug response is adverse drug reaction in statin therapy and said class specific genomic markers is selected from the group consisting of SEQ ID NO:1-SEQ ID NO:35.
7. Method of count 5 or 6 wherein said adverse drug reactions are myopathies and/or rhabdomyelosis and/or elevated creatine kinase levels.
8. Method of count 6 or 7, comprising the steps of
   i) determining the creatine kinase serum activity of a patient, wherein a patient having a creatine kinase serum activity of >80 is defined as being prone to statin adverse drug reactions; and wherein
   ii) for the remaining patients the LDL serum level, HDL serum level, cholesterin serum level and/or alkaline phosphatase serum level is determined, wherein a patient showing
      a) an LDL level of <=171 and having the SNPs as defined by SEQ ID NO: 31-33, and/or
      b) an HDL level of <=59 and having the SNPs as defined by SEQ ID NO: 34-35, and/or
      c) an cholesterol serum level of <=266 and having the SNPs as defined by SEQ ID NO: 31-33, and/or
      d) an alkaline phosphatase serum activity of >=103 and having the SNPs as defined by SEQ ID NO: 9-11, and/or
      e) an alkaline phosphatase serum activity of >=103 and having the SNPs as defined by SEQ ID NO: 6,
   is defined as being prone to statin adverse drug reactions; and wherein
   iii) remaining patients are screened for the presence of SNPs as defined by SEQ ID NO: 1-35, wherein a patient showing the SNPs as defined by SEQ ID NO: 1-35 is defined as being prone to statin adverse drug reactions.
9. Method of count 8, comprising the steps of
   i) determining the creatine kinase serum activity of a patient, wherein a patient having a creatine kinase serum activity of >70 is defined as being prone to statin adverse drug reactions; and wherein
   ii) for the remaining patients the LDL serum level, HDL serum level, cholesterin serum level and/or alkaline phosphatase serum level is determined, wherein a patient showing
      a) an LDL level of <=190 and having the SNPs as defined by SEQ ID NO: 31-33, and/or
      b) an HDL level of <=70 and having the SNPs as defined by SEQ ID NO: 34-35, and/or
      c) an cholesterol serum level of <=290 and having the SNPs as defined by SEQ ID NO: 31-33, and/or
      d) an alkaline phosphatase serum activity of >=90 and having the SNPs as defined by SEQ ID NO: 9-11, and/or
      e) an alkaline phosphatase serum activity of >=90 and having the SNPs as defined by SEQ ID NO: 6,
   is defined as being prone to statin adverse drug reactions; and wherein
   iii) remaining patients are screened for the presence of SNPs as defined by SEQ ID NO: 1-35, wherein a patient showing the SNPs as defined by SEQ ID NO: 1-35 is defined as being prone to statin adverse drug reactions.
10. Method of selecting a drug for a patient having hypercholisterinaemia, wherein statin drug response is predicted, and statin therapy or an alternative therapy is selected based on the outcome of the prediction, wherein the method of count 8 or 9 is used for statin drug response prediction, and patient still remaining after step iii) of count 8 or 9 are given statin therapy and patients defined as being prone to statin drug response should be assigned by the treating physician an alternative therapy.
11. Kit, suitable for performing a method according to counts 1-9.
12. Method according to count 8 or 9, wherein the single nucleotide polymorphisms as defined by SEQ ID NOs: 1-35 are detected on nucleotide basis.
13. Method according to count 8 or 9, wherein the polymorphisms as defined by SEQ ID NOs: 1-35 are defined on polypeptide or protein basis.
14. Method according to count 12 or 13, wherein at least one polymorphism-specific antibody specific for a polymorphism as defined by SEQ ID NOs: 1-35 is used.
15. Polymorphism-specific antibody, characterised in that the antibody is specific for a polymorphism selected from the group of polymorphisms as defined by SEQ ID NOs: 1-35.

### FIGURES

FIG. 1 shows the sequences of the SNPs of the invention, links the SNPs to the corresponding genes and to the SEQ ID NOs.

FIG. 2 shows schematically the overall workflow of the method of predicting statin adverse drug response.

FIG. 3 shows the 1^{st} and 2^{nd} step of the workflow to predict statin induced ADR

FIG. 4 shows the 3^{rd} step of the workflow to predict statin induced ADR

FIG. 5 (A-D) details a computer program which is necessary to conduct the 3^{rd} step of the workflow to predict statin induced ADR

### DETAILED DESCRIPTION OF THE INVENTION

The present invention is based at least in part on the discovery that a specific allele of a polymorphic region of a so called “candidate gene” (as defined below) is associated with an individuals response to a drug of the statin class. In order to predict those ADR other clinical parameters like the serum alkaline phosphatase levels of a patient may be of aid. Ultimately the combination of clinical serum parameters and genetic variations is helpful to predict SADR.

For the present invention the following candidate genes were analyzed:
Genes found to be expressed in cardiac tissue (Hwang et al., Circulation 1997, 96:4146-4203).
Genes from the following metabolic pathways and their regulatory elements:

### Lipid Metabolism

Numerous studies have shown a connection between serum lipid levels and cardiovascular diseases. Candidate genes falling into this group include but are not limited to genes of the cholesterol pathway, apolipoproteins and their modifying factors. As drugs of the statin class specifically target the pathway of lipid metabolism, genetic variations in those genes might influence the effect of statins on a patient.

### Drug Metabolism/ADME

The response to statin drugs is tightly linked to their bioavailability. Hence genes involved in absorption, distribution, metabolism and excretion (ADME) of drugs may be responsible for beneficial and adverse responses to statin treatment. Those genes include but are not limited to the cytochrome P450 system (e.g. CYP3A4, CYP2C9, CYP2C8), which have been shown to be involved in statin metabolism.

### Cell Structure/Motility

As it has been observed that statin treatment can lead to muscle related adverse events, genes involved in cell/muscle structure can also modulate adverse reactions to statins.

### Glucose and Energy Metabolism

As glucose and energy metabolism is interdependent with the metabolism of lipids (see above) also the former pathways contain candidate genes.

### Unclassified Genes

As stated above, the mechanisms that define the patient's individual response to drugs are not completely elucidated. Hence also candidate genes were analysed, which could not be assigned to the above listed categories. The present invention is based at least in part on the discovery of polymorphisms that lie in genomic regions of ill defined physiological function.

### Results

After conducting an association study, we surprisingly found polymorphic sites in a number of candidate genes which show a strong correlation with the response to statin medication. In detail gene variations and clinical parameters were found that could distinguish between “Tolerant patients” and “ADR patients”. “Tolerant patient” refers to individuals who can tolerate high doses of a medicament without exhibiting adverse drug reactions. “ADR patient” as used herein refers to individuals who suffer from ADR or show clinical symptoms (like creatine kinase elevation in blood) even after receiving only minor doses of a medicament (see Table 3) for a detailed definition of drug response phenotypes). As both clinical parameters and genetic variations are independent of statin treatment those variables could be assessed before onset of medication: If those parameters were found to be associated with statin ADR, alternative medications could be selected, and hence SADR could be efficiently avoided.

Polymorphic sites in candidate genes that were found to be significantly associated with SADR will be referred to as “SADR SNPs”. The respective genomic loci that harbour SADR SNPs will be referred to as “SADR genes”, irrespective of the actual biological function of this gene locus.

In particular we surprisingly found SNPs associated with statin induced adverse drug reactions (SADR) in the following genes listed in table 1:

TABLE 1
Genes identified with SNPs linked to statin induced adverse drug reactions

| HNF4A | Gene name: HNF4A |
|---|---|
| | Gene description: hepatocyte nuclear factor 4, alpha |
| | Gene aliases: TCF; HNF4; MODY; MODY1; NR2A1; TCF14; HNF4a7; |
| | HNF4a8; HNF4a9; NR2A21; FLJ39654 |
| | Summary: The protein encoded by this gene is a nuclear transcription factor |
| | which binds DNA as a homodimer. The encoded protein controls the |
| | expression of several genes, including hepatocyte nuclear factor 1 alpha, a |
| | transcription factor which regulates the expression of several hepatic genes. |
| | This gene may play a role in development of the liver, kidney, and intestines. |
| | Mutations in this gene have been associated with monogenic autosomal |
| | dominant non-insulin-dependent diabetes mellitus type I. Alternative splicing of |
| | this gene results in multiple transcript variants. |
| BAT3 | Gene name: BAT3 |
| | Gene description: HLA-B associated transcript 3 |
| | Gene aliases: G3; D6S52E |
| | Summary: A cluster of genes, BAT1-BAT5, has been localized in the vicinity |
| | of the genes for TNF alpha and TNF beta. These genes are all within the human |
| | major histocompatibility complex class III region. The protein encoded by this |
| | gene is a nuclear protein. It has been implicated in the control of apoptosis and |
| | regulating heat shock protein. There are three alternatively spliced transcript |
| | variants described for this gene. |
| CYP2C8 | Gene name: CYP2C8 |
| | Gene description: cytochrome P450, family 2, subfamily C, polypeptide 8 |
| | Gene aliases: CPC8; P450 MP-12/MP-20 |
| | Summary: This gene encodes a member of the cytochrome P450 superfamily of |
| | enzymes. The cytochrome P450 proteins are monooxygenases which catalyze |
| | many reactions involved in drug metabolism and synthesis of cholesterol, |
| | steroids and other lipids. This protein localizes to the endoplasmic reticulum |
| | and its expression is induced by phenobarbital. The enzyme is known to |
| | metabolize many xenobiotics, including the anticonvulsive drug mephenytoin, |
| | benzo(a)pyrene, 7-ethyoxycoumarin, and the anti-cancer drug taxol. Two |
| | transcript variants for this gene have been described; it is thought that the |
| | longer form does not encode an active cytochrome P450 since its protein |
| | product lacks the heme binding site. This gene is located within a cluster of |
| | cytochrome P450 genes on chromosome 10q24. |
| NDUFAB1 | Gene name: NDUFAB1 |
| | Gene description: NADH dehydrogenase (ubiquinone) 1, alpha/beta |
| | subcomplex, 1, 8 kDa |
| | Gene aliases: ACP; SDAP; MGC65095 |
| | The NADH: ubiquinone oxidoreductase (complex 1), provides the input to the |
| | respiratory chain from the NAD-linked dehydrogenases of the citric acid cycle. |
| | The complex couples the oxidation of NADH and the reduction of ubiquinone, |
| | to the generation of a proton gradient which is then used for ATP synthesis. |
| | The complex occurs in the mitochondria of eukaryotes. Mutations in this |
| | complex are associated with many disease conditions. |
| ATP1A2 | Gene name: ATP1A2 |
| | Gene description: ATPase, Na+/K+ transporting, alpha 2 (+) polypeptide |
| | Gene aliases: FHM2; MHP2; MGC59864 |
| | Summary: The protein encoded by this gene belongs to the family of P-type |
| | cation transport ATPases, and to the subfamily of Na+/K+-ATPases. Na+/K+- |
| | ATPase is an integral membrane protein responsible for establishing and |
| | maintaining the electrochemical gradients of Na and K ions across the plasma |
| | membrane. These gradients are essential for osmoregulation, for sodium- |
| | coupled transport of a variety of organic and inorganic molecules, and for |
| | electrical excitability of nerve and muscle. This enzyme is composed of two |
| | subunits, a large catalytic subunit (alpha) and a smaller glycoprotein subunit |
| | (beta). The catalytic subunit of Na+/K+-ATPase is encoded by multiple genes. |
| | This gene encodes an alpha 2 subunit. |
| HMGCS2 | Gene name: HMGCS2 |
| | Gene description: 3-hydroxy-3-methylglutaryl-Coenzyme A synthase 2 |
| | (mitochondrial) |
| | Mitochondrial 3-hydroxy-3-methylglutaryl-CoA synthase (mHMGS: EC |
| | 4.1.3.5) catalyses the first step of ketogenesis from acetyl-CoA and acetoacetyl- |
| | CoA and is considered to be the main control step in ketogenesis. The human |
| | protein is encoded by the HMGCS2 gene, which spans 20 kb genomic DNA on |
| | chromosome 1p13-p12 and contains 10 exons. mHMGS is expressed mainly in |
| | the liver and testis and is absent in other body cells. |
| APOD | Gene name: APOD |
| | Gene description: apolipoprotein D |
| | Summary: Apolipoprotein D (Apo-D) is a component of high density |
| | lipoprotein that has no marked similarity to other apolipoprotein sequences. It |
| | has a high degree of homology to plasma retinol-binding protein and other |
| | members of the alpha 2 microglobulin protein superfamily of carrier proteins, |
| | also known as lipocalins. It is a glycoprotein of estimated molecular weight 33 KDa. |
| | Apo-D is closely associated with the enzyme lecithin: cholesterol |
| | acyltransferase-an enzyme involved in lipoprotein metabolism. |
| XDH | Gene type: protein coding |
| | Gene name: XDH |
| | Gene description: xanthine dehydrogenase |
| | Gene aliases: XO; XOR |
| | Summary: Xanthine dehydrogenase belongs to the group of molybdenum- |
| | containing hydroxylases involved in the oxidative metabolism of purines. The |
| | enzyme is a homodimer. Xanthine dehydrogenase can be converted to xanthine |
| | oxidase by reversible sulfhydryl oxidation or by irreversible proteolytic |
| | modification. Defects in xanthine dehydrogenase cause xanthinuria, may |
| | contribute to adult respiratory stress syndrome, and may potentiate influenza |
| | infection through an oxygen metabolite-dependent mechanism. |
| LCAT | Gene type: protein coding |
| | Gene name: LCAT |
| | Gene description: lecithin-cholesterol acyltransferase |
| | Summary: This gene encodes the extracellular cholesterol esterifying enzyme, |
| | lecithin-cholesterol acyltransferase. The esterification of cholesterol is required |
| | for cholesterol transport. Mutations in this gene have been found to cause fish- |
| | eye disease as well as LCAT deficiency. |
| PMVK | Gene type: protein coding |
| | Gene name: PMVK |
| | Gene description: phosphomevalonate kinase |
| | Gene aliases: PMK; PMKA; PMKASE; HUMPMKI |
| | Summary: PMVK (EC 2.7.4.2) is a peroxisomal enzyme that catalyzes the |
| | conversion of mevalonate 5-phosphate into mevalonate 5-diphosphate as the |
| | fifth reaction of the cholesterol biosynthetic pathway. |
| NDUFV1 | Gene type: protein coding |
| | Gene name: NDUFV1 |
| | Gene description: NADH dehydrogenase (ubiquinone) flavoprotein 1, 51 kDa |
| | Gene aliases: UQOR1 |
| | The NNDH: ubiquinone oxidoreductase (complex 1), provides the input to the |
| | respiratory chain from the NAD-linked dehydrogenases of the citric acid cycle. |
| | The complex couples the oxidation of NADH and the reduction of ubiquinone, |
| | to the generation of a proton gradient which is then used for ATP synthesis. |
| | The complex occurs in the mitochondria of eukaryotes. Mutations in this |
| | complex are associated with many disease conditions. |
| TRIM28 | Gene type: protein coding |
| | Gene name: TRIM28 |
| | Gene description: tripartite motif-containing 28 |
| | Gene aliases: KAP1; TE1B; RNF96; TIF1B |
| | Summary: The protein encoded by this gene mediates transcriptional control by |
| | interaction with the Kruppel-associated box repression domain found in many |
| | transcription factors. The protein localizes to the nucleus and is thought to |
| | associate with specific chromatin regions. The protein is a member of the |
| | tripartite motif family. This tripartite motif includes three zinc-binding |
| | domains, a RING, a B-box type 1 and a B-box type 2, and a coiled-coil region. |
| PAK1 | Gene type: protein coding |
| | Gene name: PAK1 |
| | Gene description: p21/Cdc42/Rac1-activated kinase 1 (STE20 homolog, yeast) |
| | Gene aliases: PAKalpha |
| | Summary: PAK proteins are critical effectors that link RhoGTPases to |
| | cytoskeleton reorganization and nuclear signaling. PAK proteins, a family of |
| | serine/threonine p21-activating kinases, include PAK1, PAK2, PAK3 and |
| | PAK4. These proteins serve as targets for the small GTP binding proteins |
| | Cdc42 and Rac and have been implicated in a wide range of biological |
| | activities. PAK1 regulates cell motility and morphology. Alternative transcripts |
| | of this gene have been found, but their full-length natures have not yet been |
| | determined. |
| CALB2 | Gene type: protein coding |
| | Gene name: CALB2 |
| | Gene description: calbindin 2, 29 kDa (calretinin) |
| | Gene aliases: CAL2 |
| | Summary: Calbindin 2 (calretinin), closely related to calbindin 1, is an |
| | intracellular calcium-binding protein belonging to the troponin C superfamily. |
| | Calbindin 1 is known to be involved in the vitamin-D-dependent calcium |
| | absorption through intestinal and renal epithelia, while the function of neuronal |
| | calbindin 1 and calbindin 2 is poorly understood. The sequence of the calbindin |
| | 2 cDNA reveals an open reading frame of 271 codons coding for a protein of |
| | 31,520 Da, and shares 58% identical residues with human calbindin 1. |
| | Calbindin 2 contains five presumably active and one presumably inactive |
| | calcium-binding domains. Comparison with the partial sequences available for |
| | chick and guinea pig calbindin 2 reveals that the protein is highly conserved in |
| | evolution. The calbindin 2 message was detected in the brain, while absent |
| | from heart muscle, kidney, liver, lung, spleen, stomach and thyroid gland. |
| | There are two additional forms of alternatively spliced calbindin 2 mRNAs |
| | encoding C-terminally truncated proteins. Exon 7 can splice to exon 9, resulting |
| | in a frame shift and a translational stop at the second codon of exon 9, and |
| | encoding calretinin-20k. Exon 7 can also splice to exon 10, resulting in a frame |
| | shift and a translational stop at codon 15 of exon 10, and encoding calretinin- |
| | 22k. The truncated proteins are able to bind calcium. |
| ADCYAP1 | Gene type: protein coding |
| | Gene name: ADCYAP1 |
| | Gene description: adenylate cyclase activating polypeptide 1 (pituitary) |
| | Gene aliases: PACAP |
| | Summary: This gene encodes adenylate cyclase activating polypeptide 1. |
| | Mediated by adenylate cyclase activating polypeptide 1 receptors, this |
| | polypeptide stimulates adenylate cyclase and subsequently increases the cAMP |
| | level in target cells. Adenylate cyclase activating polypeptide 1 is not only a |
| | hypophysiotropic hormone, but also functions as a neurotransmitter and |
| | neuromodulator. In addition, it plays a role in paracrine and autocrine |
| | regulation of certain types of cells. This gene is composed of five exons. Exons |
| | 1 and 2 encode the 5′ UTR and signal peptide, respectively; exon 4 encodes an |
| | adenylate cyclase activating polypeptide 1-related peptide; and exon 5 encodes |
| | the mature peptide and 3′ UTR. This gene encodes three different mature |
| | peptides, including two isotypes: a shorter form and a longer form. |
| PRKAR1A | Gene type: protein coding |
| | Gene name: PRKAR1A |
| | Gene description: protein kinase, cAMP-dependent, regulatory, type I, alpha |
| | (tissue specific extinguisher 1) |
| | Gene aliases: CAR; CNC1; PKR1; TSE1; PRKAR1; MGC17251; |
| | DKFZp779L0468 |
| | Summary: cAMP is a signaling molecule important for a variety of cellular |
| | functions. cAMP exerts its effects by activating the cAMP-dependent protein |
| | kinase (AMPK), which transduces the signal through phosphorylation of |
| | different target proteins. The inactive holoenzyme of AMPK is a tetramer |
| | composed of two regulatory and two catalytic subunits. cAMP causes the |
| | dissociation of the inactive holoenzyme into a dimer of regulatory subunits |
| | bound to four cAMP and two free monomeric catalytic subunits. Four different |
| | regulatory subunits and three catalytic subunits of AMPK have been identified |
| | in humans. The protein encoded by this gene is one of the regulatory subunits. |
| | This protein was found to be a tissue-specific extinguisher that down-regulates |
| | the expression of seven liver genes in hepatoma x fibroblast hybrids. Functional |
| | null mutations in this gene cause Carney complex (CNC), an autosomal |
| | dominant multiple neoplasia syndrome. This gene can fuse to the RET |
| | protooncogene by gene rearrangement and form the thyroid tumor-specific |
| | chimeric oncogene known as PTC2. Three alternatively spliced transcript |
| | variants encoding the same protein have been observed. |
| NF1 | Gene type: protein coding |
| | Gene name: NF |
| | Gene description: neurofibromin 1 (neurofibromatosis, von Recklinghausen |
| | disease, Watson disease) |
| | Gene aliases: WSS; NFNS; VRNF; DKFZp686J1293 |
| | Summary: Mutations linked to neurofibromatosis type 1 led to the identification |
| | of NF1. NF1 encodes the protein neurofibromin, which appears to be a negative |
| | regulator of the ras signal transduction pathway. In addition to type 1 |
| | neurofibromatosis, mutations in NF1 can also lead to juvenile myelomonocytic |
| | leukemia. Alternatively spliced NF1 mRNA transcripts have been isolated, |
| | although their functions, if any, remain unclear. |

As SADR SNPs are linked to other SNPs in neighboring genes on a chromosome (Linkage Disequilibrium) those SNPs could also be used as marker SNPs. In a recent publication it was shown that SNPs are linked over 100 kb in some cases more than 150 kb (Reich D. E. et al. Nature 411, 199-204, 2001). Hence SNPs lying in regions neighbouring SADR SNPs could be linked to the latter and by this being a diagnostic marker. These associations could be performed as described for the gene polymorphism in methods.

TABLE 2
Clinical parameters and unit definitions

| Clinical Parameter | Abreviation | Unit definition and limit values |
|---|---|---|
| Creatine Kinase | CK | U/I* (measured at 25° C.) |
| | | Upper limit of normal: ♀ 70 U/I, |
| | | ♂ 80 U/I |
| Low Density | LDL | mg/dl |
| Lipoprotein | | |
| High Densitiy | HDL | mg/dl |
| Lipoprotein | | |
| Cholesterol | CHOL | mg/dl |
| Alkaline Phosphatase | ALP | U/I* (measured at 25° C.) |
| | | Upper limit of normal: ♀ + |
| | | ♂: 60-170 U/I |

*1 U = 16,67 nkat

### Methods for Assessing a Patient's Tolerability to Statin Drugs

The present invention provides diagnostic methods for assessing the predisposition of a patient for statin adverse drug reaction (SADR). It will be understood that a diagnosis of predisposition to statin ADR made by a medical practitioner encompasses clinical measurements and medical judgement. Predisposition markers according to the invention are assessed using conventional methods well known in the art. Statin adverse drug reactions include, among others, myopathies and/or rhabdomyelosis.

The methods are carried out by the steps of:
i) determining the creatine kinase serum activity of a patient, wherein a patient having a creatine kinase serum activity of >80 is defined as being prone to statin adverse drug reactions; and wherein
ii) for the remaining patients the LDL serum level, HDL serum level, cholesterin serum level and/or alkaline phosphatase serum level is determined, wherein a patient showing
   a) an LDL level of <=171 and having the SNPs as defined by SEQ ID NO: 31-33, and/or
   b) an HDL level of <=59 and having the SNPs as defined by SEQ ID NO: 34-35, and/or
   c) an cholesterol serum level of <=266 and having the SNPs as defined by SEQ ID NO: 31-33, and/or
   d) an alkaline phosphatase serum activity of >=103 and having the SNPs as defined by SEQ ID NO: 9-11, and/or
   e) an alkaline phosphatase serum activity of >=103 and having the SNPs as defined by SEQ ID NO: 6,
      is defined as being prone to statin adverse drug reactions; and wherein
iii) remaining patients are screened for the presence of SNPs as defined by SEQ ID NO: 1-35, wherein a patient showing the SNPs as defined by SEQ ID NO: 1-35 is defined as being prone to statin adverse drug reactions.

An alternative method comprises the steps of
i) determining the creatine kinase serum activity of a patient, wherein a patient having a creatine kinase serum activity of >70 is defined as being prone to statin adverse drug reactions; and wherein
ii) for the remaining patients the LDL serum level, HDL serum level, cholesterin serum level and/or alkaline phosphatase serum level is determined, wherein a patient showing
   a) an LDL level of <=190 and having the SNPs as defined by SEQ ID NO: 31-33, and/or
   b) an HDL level of <=70 and having the SNPs as defined by SEQ ID NO: 34-35, and/or
   c) an cholesterol serum level of <=290 and having the SNPs as defined by SEQ ID NO: 31-33, and/or
   d) an alkaline phosphatase serum activity of >=90 and having the SNPs as defined by SEQ ID NO: 9-11, and/or
   e) an alkaline phosphatase serum activity of >=90 and having the SNPs as defined by SEQ ID NO: 6,
      is defined as being prone to statin adverse drug reactions; and wherein
iii) remaining patients are screened for the presence of SNPs as defined by SEQ ID NO: 1-35, wherein a patient showing the SNPs as defined by SEQ ID NO: 1-35 is defined as being prone to statin adverse drug reactions.

FIG. 2 shows schematically the overall workflow of the method of predicting statin adverse drug response. “Case” means a patient identified as being prone to statin adverse drug response. “CK” means serum creatine kinase levels.

In another embodiment, the method involves comparing an individual's polymorphic pattern with polymorphic patterns of individuals who exhibit or have exhibited one or more drug related phenotypes, such as adverse drug reactions.

In practicing the methods of the invention, an individual's polymorphic pattern can be established by obtaining DNA from the individual and determining the sequence at predetermined polymorphic positions in the genes such as those described in this file.

The DNA may be obtained from any cell source. Non-limiting examples of cell sources available in clinical practice include blood cells, buccal cells, cervicovaginal cells, epithelial cells from urine, fetal cells, or any cells present in tissue obtained by biopsy. Cells may also be obtained from body fluids, including without limitation blood, saliva, sweat, urine, cerebrospinal fluid, feces, and tissue exudates at the site of infection or inflammation. DNA is extracted from the cell source or body fluid using any of the numerous methods that are standard in the art. It will be understood that the particular method used to extract DNA will depend on the nature of the source.

### Diagnostic and Prognostic Assays

The present invention provides methods for determining the molecular structure of at least one polymorphic region of a gene, specific allelic variants of said polymorphic region being associated with SADR.

In one embodiment, determining the molecular structure of a polymorphic region of a gene comprises determining the identity of the allelic variant. A polymorphic region of a gene, of which specific alleles are associated with statin induced ADR can be located in an exon, an intron, at an intron/exon border, or in the promoter of the gene.

The invention provides methods for determining whether a subject has, or is at risk, of developing SADR. Such disorder can be associated with an aberrant gene activity, e.g., abnormal binding to a form of a lipid, or an aberrant gene protein level. An aberrant gene protein level can result from an aberrant transcription or post-transcriptional regulation. Thus, allelic differences in specific regions of a gene can result in differences of gene protein due to differences in regulation of expression. In particular, some of the identified polymorphisms in the human gene may be associated with differences in the level of transcription, RNA maturation, splicing, or translation of the gene or transcription product.

In preferred embodiments, the methods of the invention can be characterized as comprising detecting, in a sample of cells from the subject, the presence or absence of a specific allelic variant of one or more polymorphic regions of a gene. The allelic differences can be: (i) a difference in the identity of at least one nucleotide or (ii) a difference in the number of nucleotides, which difference can be a single nucleotide or several nucleotides.

A preferred detection method is allele specific hybridization using probes overlapping the polymorphic site and having about 5, 10, 20, 25, or 30 nucleotides around the polymorphic region. Examples of probes for detecting specific allelic variants of the polymorphic region located in a SADR gene are probes comprising a nucleotide sequence set forth in any of SEQ ID NO. 1-35. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism of Seq ID 1 and that of other possible polymorphic regions can be determined in a single hybridization experiment.

In other detection methods, it is necessary to first amplify at least a portion of a gene prior to identifying the allelic variant. Amplification can be performed, e.g., by polymerase chain reaction (PCR) and/or ligase chain reaction (LCR), according to methods known in the art. In one embodiment, genomic DNA of a cell is exposed to two PCR primers and amplification for a number of cycles sufficient to produce the required amount of amplified DNA. In preferred embodiments, the primers are located between 40 and 350 base pairs apart.

Alternative amplification methods include: self sustained sequence replication (Guatelli, J. C. et al., 1990, Proc. Natl. Acad. Sci. U.S.A. 87:1874-1878), transcriptional amplification system (Kwoh, D. Y. et al., 1989, Proc. Natl. Acad. Sci. U.S.A. 86:1173-1177), Q-Beta Replicase (Lizardi, P. M. et al., 1988, Bio/Technology 6:1197), whole genome amplification (WGA) or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a gene and detect allelic variants, e.g., mutations, by comparing the sequence of the sample sequence with the corresponding wild-type (control) sequence. Exemplary sequencing reactions include those based on techniques developed by Maxam and Gilbert (Proc. Natl. Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a gene in DNA from a subject can be shown by restriction enzyme analysis. For example, a specific nucleotide polymorphism can result in a nucleotide sequence comprising a restriction site which is absent from the nucleotide sequence of another allelic variant.

In other embodiments, alterations in electrophoretic mobility are used to identify the type of gene allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl. Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of gene. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each LA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a gene.

Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990), Kuppuswamy, M. N. et al., Proc. Natl. Acad. Sci. (U.S.A.) 88:1143-1147 (1991); Prezant, T. R. et al., Hum. Mutat. 1: 159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For determining the identity of the allelic variant of a polymorphic region located in the coding region of a gene, yet other methods than those described above can be used. For example, identification of an allelic variant which encodes a mutated gene protein can be performed by using an antibody specifically recognizing the mutant protein in, e.g., immunohistochemistry or immunoprecipitation. Antibodies to wild-type gene protein are described, e.g., in Acton et al. (1999) Science 271:518 (anti-mouse gene antibody cross-reactive with human gene). Other antibodies to wild-type gene or mutated forms of gene proteins can be prepared according to methods known in the art. Alternatively, one can also measure an activity of a gene protein, such as binding to a lipid or lipoprotein. Binding assays are known in the art and involve, e.g., obtaining cells from a subject, and performing binding experiments with a labeled lipid, to determine whether binding to the mutated form of the receptor differs from binding to the wild-type of the receptor.

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and SSCP.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific gene allelic variant.

Sample nucleic acid for using in the above-described diagnostic and prognostic methods can be obtained from any cell type or tissue of a subject. For example, a subject's bodily fluid (e.g. blood) can be obtained by known techniques (e.g. venipuncture) or from human tissues like heart (biopsies, transplanted organs). Alternatively, nucleic acid tests can be performed on dry samples (e.g. hair or skin). Fetal nucleic acid samples for prenatal diagnostics can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, New York).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

### Advantage of the Invention

For example the present invention can identify patients exhibiting a combination of clinical parameters and genetic polymorphisms which indicate an increased risk for statin induced adverse drug reactions. In that case the drug dose should be lowered in a way that the risk for SADR is diminished.

It is self evident that the ability to predict a patient's individual drug response should affect the formulation of a drug, i.e. drug formulations should be tailored in a way that they suit the different patient classes (low/high responder, poor/good metabolizer, ADR prone patients). Those different drug formulations may encompass different doses of the drug, i.e. the medicinal products contain low or high amounts of the active substance. In another embodiment of the invention the drug formulation may contain additional substances that facilitate the beneficial effects and/or diminish the risk for ADR (Folkers et al. 1991, U.S. Pat. No. 5,316,765).

### Isolated Polymorphic Nucleic Acids, Probes, and Vectors

The present invention provides isolated nucleic acids comprising the polymorphic positions described herein for human genes; vectors comprising the nucleic acids; and transformed host cells comprising the vectors. The invention also provides probes which are useful for detecting these polymorphisms.

In practicing the present invention, many conventional techniques in molecular biology, microbiology, and recombinant DNA, are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Ausubel et al., Current Protocols in Molecular Biology, 1997, (John Wiley and Sons); and Methods in Enzymology Vol. 154 and Vol. 155 (Wu and Grossman, and Wu, eds., respectively).

Insertion of nucleic acids (typically DNAs) comprising the sequences in a functional surrounding like full length cDNA of the present invention into a vector is easily accomplished when the termini of both the DNAs and the vector comprise compatible restriction sites. If this cannot be done, it may be necessary to modify the termini of the DNAs and/or vector by digesting back single-stranded DNA overhangs generated by restriction endonuclease cleavage to produce blunt ends, or to achieve the same result by filling in the single-stranded termini with an appropriate DNA polymerase.

Alternatively, any site desired may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., 1988, Science 239:48. The cleaved vector and the DNA fragments may also be modified if required by homopolymeric tailing.

The nucleic acids may be isolated directly from cells or may be chemically synthesized using known methods. Alternatively, the polymerase chain reaction (PCR) method can be used to produce the nucleic acids of the invention, using either chemically synthesized strands or genomic material as templates. Primers used for PCR can be synthesized using the sequence information provided herein and can further be designed to introduce appropriate new restriction sites, if desirable, to facilitate incorporation into a given vector for recombinant expression.

The nucleic acids of the present invention may be flanked by native gene sequences, or may be associated with heterologous sequences, including promoters, enhancers, response elements, signal sequences, polyadenylation sequences, introns, 5′- and 3′-noncoding regions, and the like. The nucleic acids may also be modified by many means known in the art. Non-limiting examples of such modifications include methylation, “caps”, substitution of one or more of the naturally occurring nucleotides with an analog, internucleotide modifications such as, for example, those with uncharged linkages (e.g., methyl phosphonates, phosphotriesters, phosphoroamidates, carbamates, morpholines etc.) and with charged linkages (e.g., phosphorothioates, phosphorodithioates, etc.). Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. PNAs are also included. The nucleic acid may be derivatized by formation of a methyl or ethyl phosphotriester or an alkyl phosphoramidate linkage. Furthermore, the nucleic acid sequences of the present invention may also be modified with a label capable of providing a detectable signal, either directly or indirectly. Exemplary labels include radioisotopes, fluorescent molecules, biotin, and the like.

The invention also provides nucleic acid vectors comprising the gene sequences or derivatives or fragments thereof of genes described in the Examples. A large number of vectors, including plasmid and fungal vectors, have been described for replication and/or expression in a variety of eukaryotic and prokaryotic hosts, and may be used for gene therapy as well as for simple cloning or protein expression. Non-limiting examples of suitable vectors include without limitation pUC plasmids, pET plasmids (Novagen, Inc., Madison, Wis.), or pRSET or pREP (Invitrogen, San Diego, Calif.), and many appropriate host cells, using methods disclosed or cited herein or otherwise known to those skilled in the relevant art. The particular choice of vector/host is not critical to the practice of the invention.

Suitable host cells may be transformed/transfected/infected as appropriate by any suitable method including electroporation, CaCl₂ mediated DNA uptake, fungal or viral infection, microinjection, microprojectile, or other established methods. Appropriate host cells included bacteria, archebacteria, fungi, especially yeast, and plant and animal cells, especially mammalian cells. A large number of transcription initiation and termination regulatory regions have been isolated and shown to be effective in the transcription and translation of heterologous proteins in the various hosts. Examples of these regions, methods of isolation, manner of manipulation, etc. are known in the art. Under appropriate expression conditions, host cells can be used as a source of recombinantly produced peptides and polypeptides encoded by genes of the Examples. Nucleic acids encoding peptides or polypeptides from gene sequences of the Examples may also be introduced into cells by recombination events. For example, such a sequence can be introduced into a cell and thereby effect homologous recombination at the site of an endogenous gene or a sequence with substantial identity to the gene. Other recombination-based methods such as non-homologous recombinations or deletion of endogenous genes by homologous recombination may also be used.

In case of proteins that form heterodimers or other multimers, both or all subunits have to be expressed in one system or cell.

The nucleic acids of the present invention find use as probes for the detection of genetic polymorphisms and as templates for the recombinant production of normal or variant peptides or polypeptides encoded by genes listed in the Examples.

Probes in accordance with the present invention comprise without limitation isolated nucleic acids of about 10-100 bp, preferably 15-75 bp and most preferably 17-25 bp in length, which hybridize at high stringency to one or more of the polymorphic sequences disclosed herein or to a sequence immediately adjacent to a polymorphic position. Furthermore, in some embodiments a full-length gene sequence may be used as a probe. In one series of embodiments, the probes span the polymorphic positions in genes disclosed herein. In another series of embodiments, the probes correspond to sequences immediately adjacent to the polymorphic positions.

### Polymorphic Polypeptides and Polymorphism-Specific Antibodies

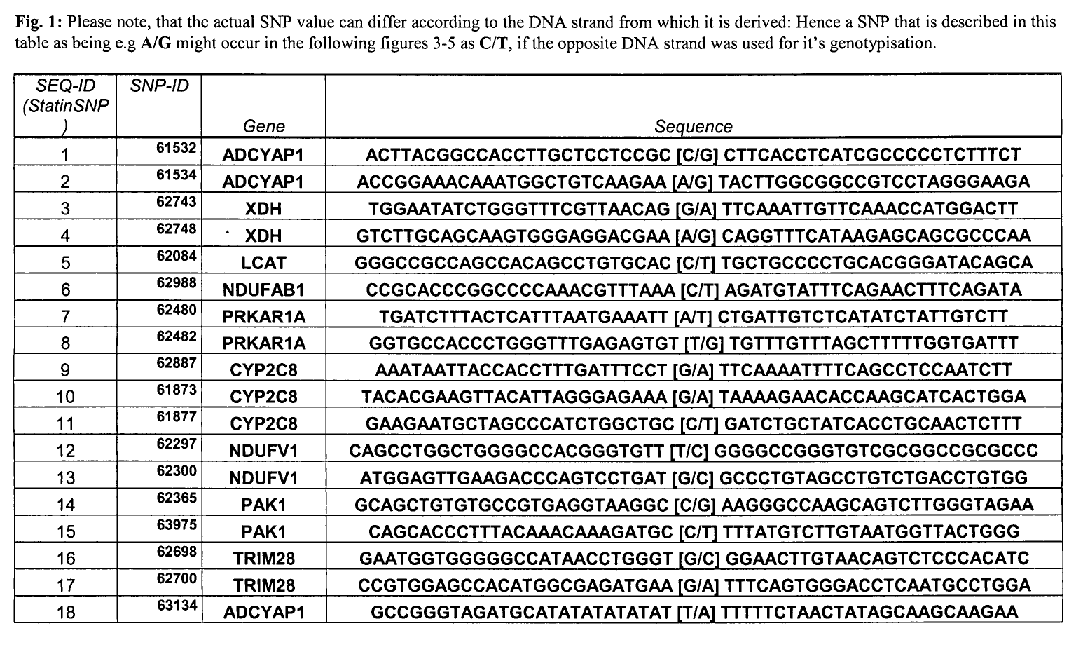

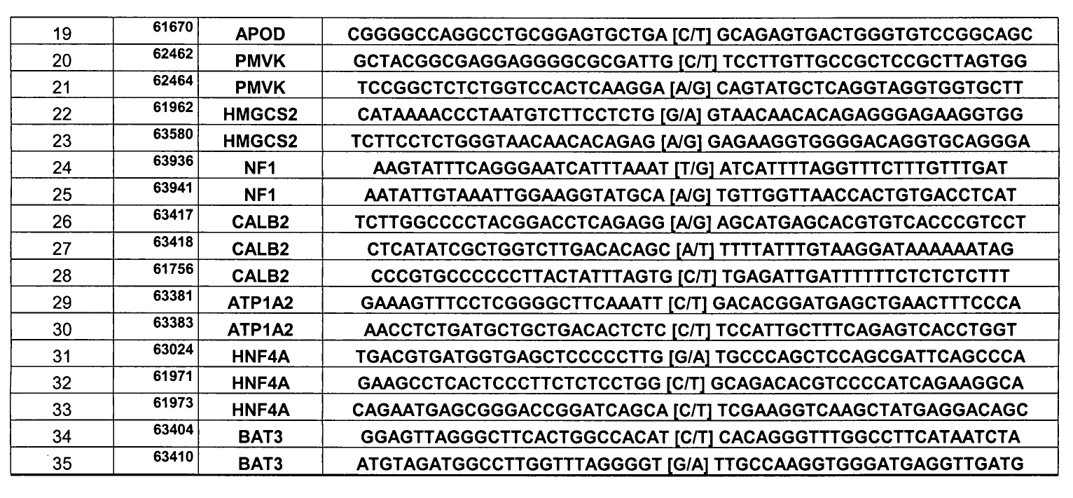

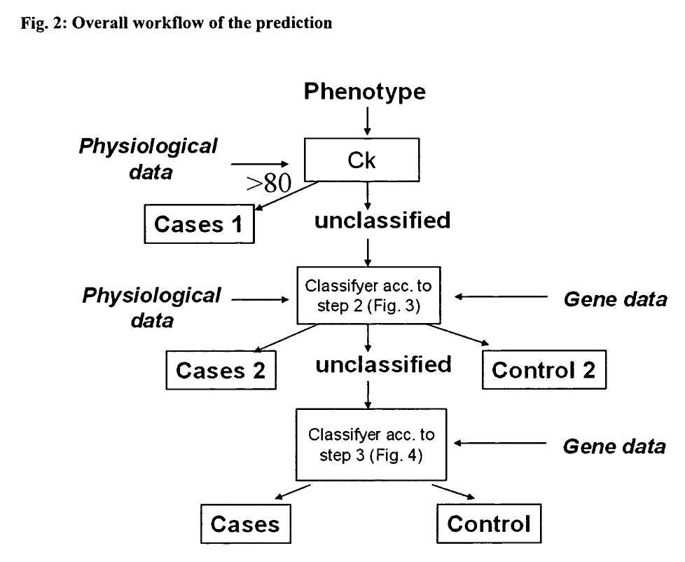

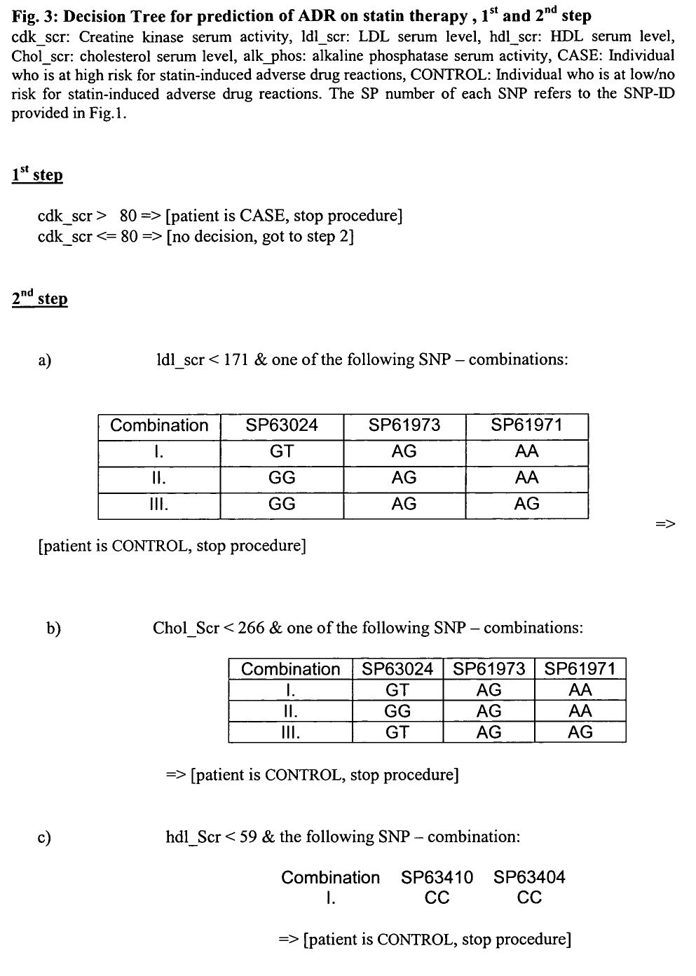

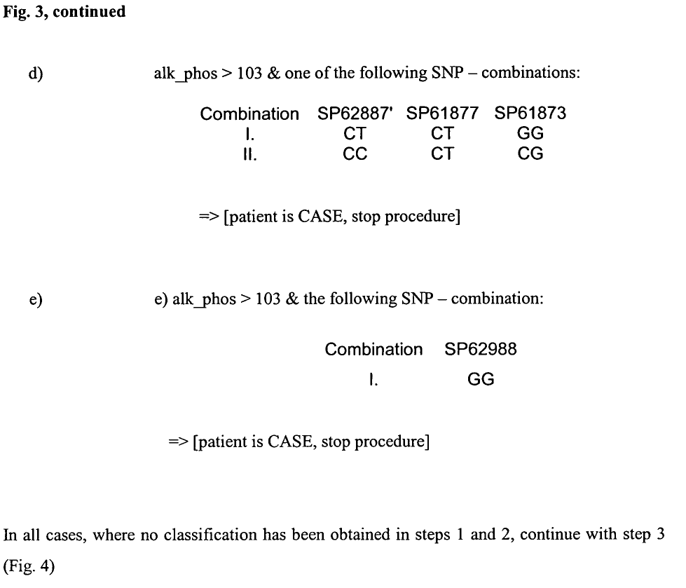

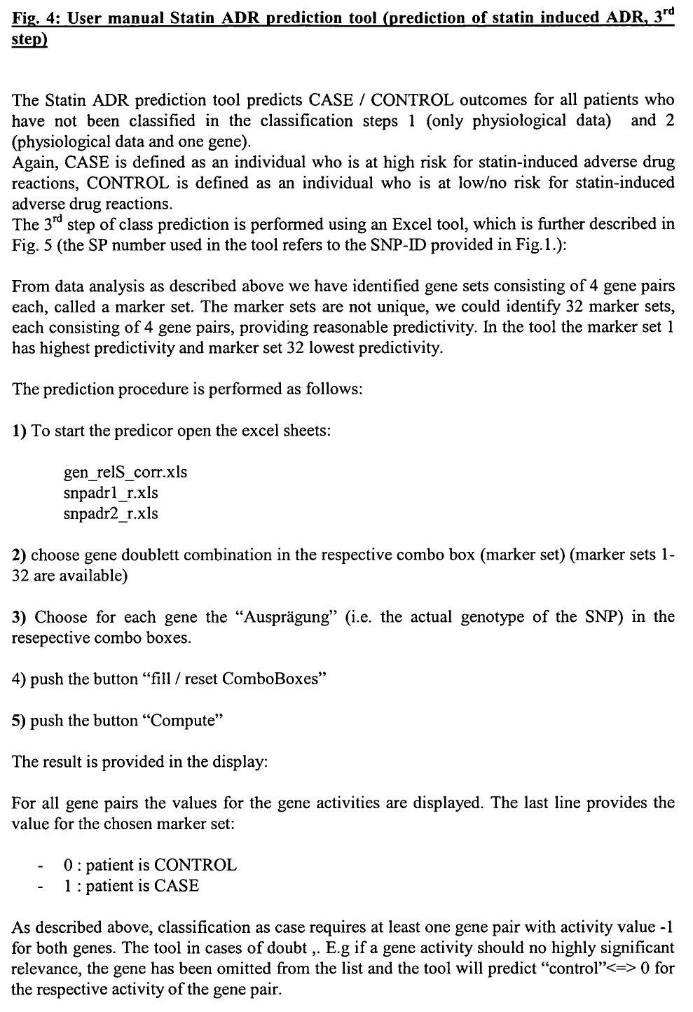

The present invention encompasses isolated peptides and polypeptides encoded by genes listed in table 1 comprising polymorphic positions disclosed herein (see e.g. FIG. 1). In one preferred embodiment, the peptides and polypeptides are useful screening targets to identify cardiovascular drugs. In another preferred embodiments, the peptides and polypeptides are capable of eliciting antibodies in a suitable host animal that react specifically with a polypeptide comprising the polymorphic position and distinguish it from other polypeptides having a different sequence at that position.

Polypeptides according to the invention are preferably at least five or more residues in length, preferably at least fifteen residues. Methods for obtaining these polypeptides are described below. Many conventional techniques in protein biochemistry and immunology are used. Such techniques are well known and are explained in Immunochemical Methods in Cell and Molecular Biology, 1987 (Mayer and Waler, eds; Academic Press, London); Scopes, 1987, Protein Purification: Principles and Practice, Second Edition (Springer-Verlag, N.Y.) and Handbook of Experimental Immunology, 1986, Volumes I-IV (Weir and Blackwell eds.).

Nucleic acids comprising protein-coding sequences can be used to direct the ITT recombinant expression of polypeptides encoded by genes disclosed herein in intact cells or in cell-free translation systems. The known genetic code, tailored if desired for more efficient expression in a given host organism, can be used to synthesize oligonucleotides encoding the desired amino acid sequences. The polypeptides may be isolated from human cells, or from heterologous organisms or cells (including, but not limited to, bacteria, fungi, insect, plant, and mammalian cells) into which an appropriate protein-coding sequence has been introduced and expressed. Furthermore, the polypeptides may be part of recombinant fusion proteins.

Peptides and polypeptides may be chemically synthesized by commercially available automated procedures, including, without limitation, exclusive solid phase synthesis, partial solid phase methods, fragment condensation or classical solution synthesis. The polypeptides are preferably prepared by solid phase peptide synthesis as described by Merrifield, 1963, J. Am. Chem. Soc. 85:2149.

Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against peptides encoded by genes disclosed herein, can be used as purification reagents. Other purification methods are possible.

The present invention also encompasses derivatives and homologues of the polypeptides. For some purposes, nucleic acid sequences encoding the peptides may be altered by substitutions, additions, or deletions that provide for functionally equivalent molecules, i.e., function-conservative variants. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids.

The isolated polypeptides may be modified by, for example, phosphorylation, sulfation, acylation, or other protein modifications. They may also be modified with a label capable of providing a detectable signal, either directly or indirectly, including, but not limited to, radioisotopes and fluorescent compounds.

The present invention also encompasses antibodies that specifically recognize the polymorphic positions of the invention and distinguish a peptide or polypeptide containing a particular polymorphism from one that contains a different sequence at that position. Such polymorphic position-specific antibodies according to the present invention include polyclonal and monoclonal antibodies. The antibodies may be elicited in an animal host by immunization with peptides encoded by genes disclosed herein or may be formed by in vitro immunization of immune cells. The immunogenic components used to elicit the antibodies may be isolated from human cells or produced in recombinant systems. The antibodies may also be produced in recombinant systems programmed with appropriate antibody-encoding DNA. Alternatively, the antibodies may be constructed by biochemical reconstitution of purified heavy and light chains. The antibodies include hybrid antibodies (i.e., containing two sets of heavy chain/light chain combinations, each of which recognizes a different antigen), chimeric antibodies (i.e., in which either the heavy chains, light chains, or both, are fusion proteins), and univalent antibodies (i.e., comprised of a heavy chain/light chain complex bound to the constant region of a second heavy chain). Also included are Fab fragments, including Fab′ and F(ab).sub.2 fragments of antibodies. Methods for the production of all of the above types of antibodies and derivatives are well-known in the art and are discussed in more detail below. For example, techniques for producing and processing polyclonal antisera are disclosed in Mayer and Walker, 1987, Immunochemical Methods in Cell and Molecular Biology, (Academic Press, London). The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. See, e.g., Schreier et al., 1980, Hybridoma Techniques; U.S. Pat. Nos. 4,341,761; 4,399,121; 4,427,783; 4,444,887; 4,466,917; 4,472,500; 4,491,632; and 4,493,890. Panels of monoclonal antibodies produced against peptides encoded by genes disclosed herein can be screened for various properties; i.e. for isotype, epitope affinity, etc.

The antibodies of this invention can be purified by standard methods, including but not limited to preparative disc-gel electrophoresis, isoelectric focusing, HPLC, reversed-phase HPLC, gel filtration, ion exchange and partition chromatography, and countercurrent distribution. Purification methods for antibodies are disclosed, e.g., in The Art of Antibody Purification, 1989, Amicon Division, W. R. Grace & Co. General protein purification methods are described in Protein Purification: Principles and Practice, R. K. Scopes, Ed., 1987, Springer-Verlag, New York, N.Y.

Methods for determining the immunogenic capability of the disclosed sequences and the characteristics of the resulting sequence-specific antibodies and immune cells are well-known in the art. For example, antibodies elicited in response to a peptide comprising a particular polymorphic sequence can be tested for their ability to specifically recognize that polymorphic sequence, i.e., to bind differentially to a peptide or polypeptide comprising the polymorphic sequence and thus distinguish it from a similar peptide or polypeptide containing a different sequence at the same position.

### Kits

As set forth herein, the invention provides diagnostic methods, e.g., for determining the identity of the allelic variants of polymorphic regions present in the gene loci of genes disclosed herein, wherein specific allelic variants of the polymorphic region are associated with cardiovascular diseases. In a preferred embodiment, the diagnostic kit can be used to determine whether a subject is at risk of developing SADR. This information could then be used, e.g., to optimize treatment of such individuals.

In preferred embodiments, the kit comprises a probe or primer which is capable of hybridizing to a gene and thereby identifying whether the gene contains an allelic variant of a polymorphic region which is associated with a risk for cardiovascular disease. The kit preferably further comprises instructions for use in diagnosing a subject as having, or having a predisposition, towards developing SADR. The probe or primers of the kit can be any of the probes or primers described in this file.

Preferred kits for amplifying a region of a gene comprising a polymorphic region of interest comprise one, two or more primers.

### Antibody-Based Diagnostic Methods and Kits:

The invention also provides antibody-based methods for detecting polymorphic patterns in a biological sample. The methods comprise the steps of: (i) contacting a sample with one or more antibody preparations, wherein each of the antibody preparations is specific for a particular polymorphic form of the proteins encoded by genes disclosed herein, under conditions in which a stable antigen-antibody complex can form between the antibody and antigenic components in the sample; and (ii) detecting any antigen-antibody complex formed in step (i) using any suitable means known in the art, wherein the detection of a complex indicates the presence of the particular polymorphic form in the sample.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

The present invention also provides kits suitable for antibody-based diagnostic applications. Diagnostic kits typically include one or more of the following components:
(i) Polymorphism-specific antibodies. The antibodies may be pre-labeled; alternatively, the antibody may be unlabelled and the ingredients for labeling may be included in the kit in separate containers, or a secondary, labeled antibody is provided; and
(ii) Reaction components: The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput and/or automated operation.

### Drug Targets and Screening Methods

According to the present invention, nucleotide sequences derived from genes disclosed herein and peptide sequences encoded by genes disclosed herein, particularly those that contain one or more polymorphic sequences, comprise useful targets to identify cardiovascular drugs, i.e., compounds that are effective in treating one or more clinical symptoms of cardiovascular disease. Furthermore, especially when a protein is a multimeric protein that are build of two or more subunits, is a combination of different polymorphic subunits very useful.

Drug targets include without limitation (i) isolated nucleic acids derived from the genes disclosed herein, and (ii) isolated peptides and polypeptides encoded by genes disclosed herein, each of which comprises one or more polymorphic positions.

### In Vitro Screening Methods:

In one series of embodiments, an isolated nucleic acid comprising one or more polymorphic positions is tested in vitro for its ability to bind test compounds in a sequence-specific manner. The methods comprise:
(i) providing a first nucleic acid containing a particular sequence at a polymorphic position and a second nucleic acid whose sequence is identical to that of the first nucleic acid except for a different sequence at the same polymorphic position;
(ii) contacting the nucleic acids with a multiplicity of test compounds under conditions appropriate for binding; and
(iii) identifying those compounds that bind selectively to either the first or second nucleic acid sequence.

Selective binding as used herein refers to any measurable difference in any parameter of binding, such as, e.g., binding affinity, binding capacity, etc.

In another series of embodiments, an isolated peptide or polypeptide comprising one or more polymorphic positions is tested in vitro for its ability to bind test compounds in a sequence-specific manner. The screening methods involve:
(i) providing a first peptide or polypeptide containing a particular sequence at a polymorphic position and a second peptide or polypeptide whose sequence is identical to the first peptide or polypeptide except for a different sequence at the same polymorphic position;
(ii) contacting the polypeptides with a multiplicity of test compounds under conditions appropriate for binding; and
(iii) identifying those compounds that bind selectively to one of the nucleic acid sequences.

In preferred embodiments, high-throughput screening protocols are used to survey a large number of test compounds for their ability to bind the genes or peptides disclosed above in a sequence-specific manner.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means.

### In Vivo Screening Methods:

Intact cells or whole animals expressing polymorphic variants of genes disclosed herein can be used in screening methods to identify candidate cardiovascular drugs.

In one series of embodiments, a permanent cell line is established from an individual exhibiting a particular polymorphic pattern. Alternatively, cells (including without limitation mammalian, insect, yeast, or bacterial cells) are programmed to express a gene comprising one or more polymorphic sequences by introduction of appropriate DNA. Identification of candidate compounds can be achieved using any suitable assay, including without limitation (i) assays that measure selective binding of test compounds to particular polymorphic variants of proteins encoded by genes disclosed herein; (ii) assays that measure the ability of a test compound to modify (i.e., inhibit or enhance) a measurable activity or function of proteins encoded by genes disclosed herein; and (iii) assays that measure the ability of a compound to modify (i.e., inhibit or enhance) the transcriptional activity of sequences derived from the promoter (i.e., regulatory) regions of genes disclosed herein.

In another series of embodiments, transgenic animals are created in which (i) one or more human genes disclosed herein, having different sequences at particular polymorphic positions are stably inserted into the genome of the transgenic animal; and/or (ii) the endogenous genes disclosed herein are inactivated and replaced with human genes disclosed herein, having different sequences at particular polymorphic positions. See, e.g., Coffman, Semin. Nephrol. 17:404, 1997; Esther et al., Lab. Invest. 74:953, 1996; Murakami et al., Blood Press. Suppl. 2:36, 1996. Such animals can be treated with candidate compounds and monitored for one or more clinical markers of cardiovascular status.

The following are intended as non-limiting examples of the invention.

### Material and Methods

Genotyping of patient DNA was performed using MALDI TOF mass spectrometry (van den Boom et al., Int J Mass Spectrometry 2004, 238(2):173-188).

### EXAMPLES

The method of predicting statin adverse drug reaction has been validated by a test run with control and case (being prone to SADR) patients. Table 3 shows the criteria used to define control and case patients.

TABLE 3
Definition of controls and patients suffering from statin
induced adverse drug reactions

| | |
|---|---|
| Control patient | No diagnosis of muscle cramps, muscle pain, |
| (good statin | muscle weakness, myalgia or myopathy after |
| onset tolerability) | of statin treatment |
| | AND |
| | serum creatine kinase (CK) levels below 70 U/I in |
| | women and below 80 U/I in men. |
| Case patient | Diagnosis of muscle cramps, muscle pain, muscle |
| (with statin induced | weakness, myalgia or myopathy |
| adverse drug reactions) | OR |
| | serum CK levels higher than 140 U/I in women |
| | and 160 U/I in men. |

An informed consent was signed by the patients and control people. Blood was taken by a physician according to medical standard procedures.

Samples were collected anonymous and labeled with a patient number.

DNA was extracted using kits from Qiagen.

### Results:

The overall specificity was 98.1% and the overall sensitivity was 80% in average test sets. The overall sensitivity in average training sets (specificity=100%) was 94%. This data were measured by cross-validation (85% training set data, 15% test set data chosen by randomized selection). Overall specificity and sensitivity data are means from the respective predictions on all test sets, where in each run the model has been trained only on the training set data.

Identification of ADR Patients (Cases) and Individuals with No Risk for ADR (Controls)

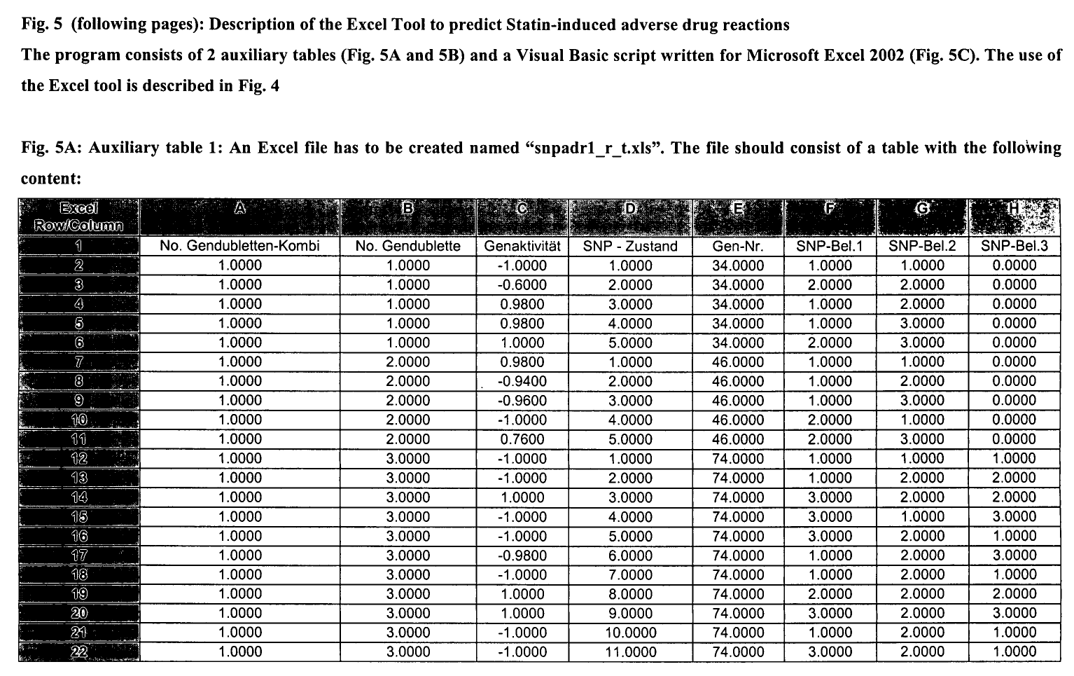

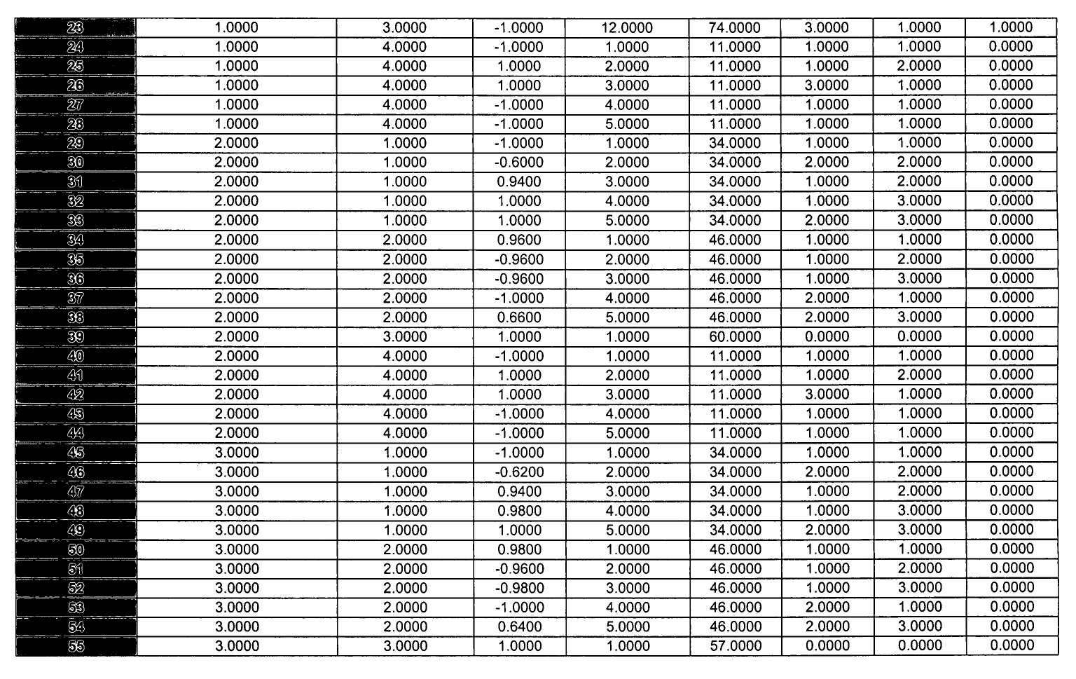

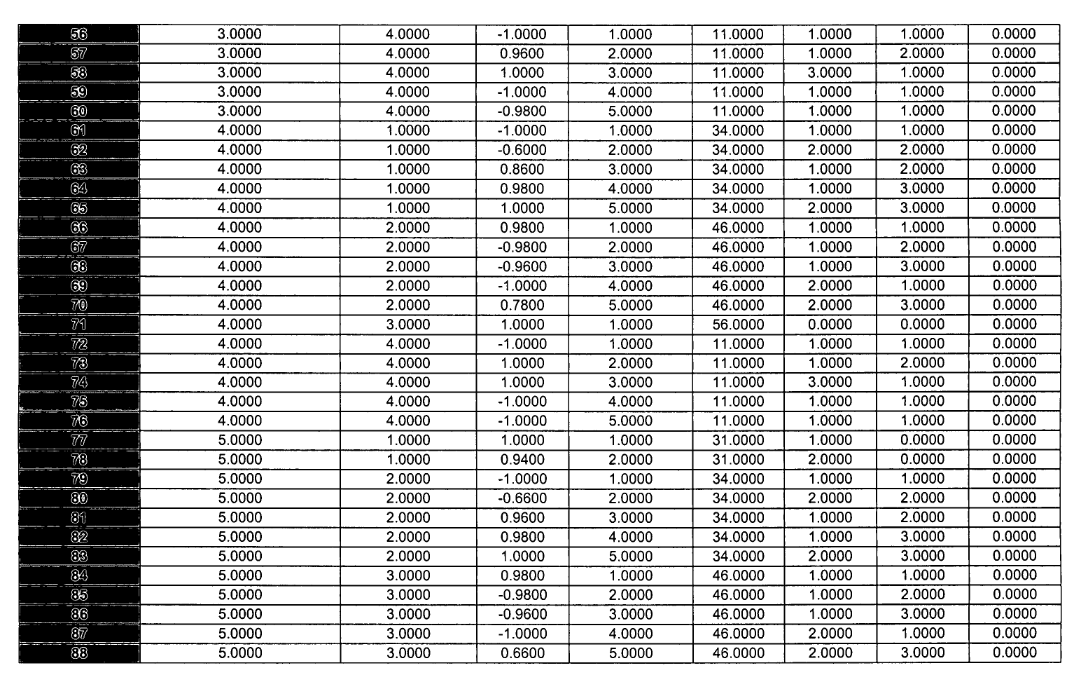

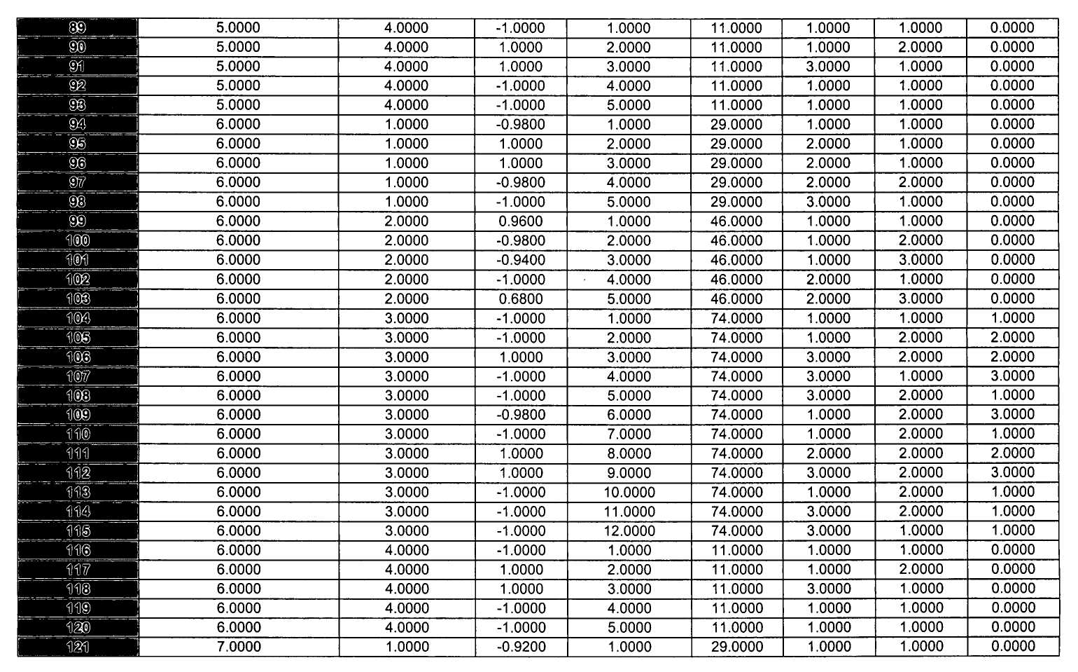

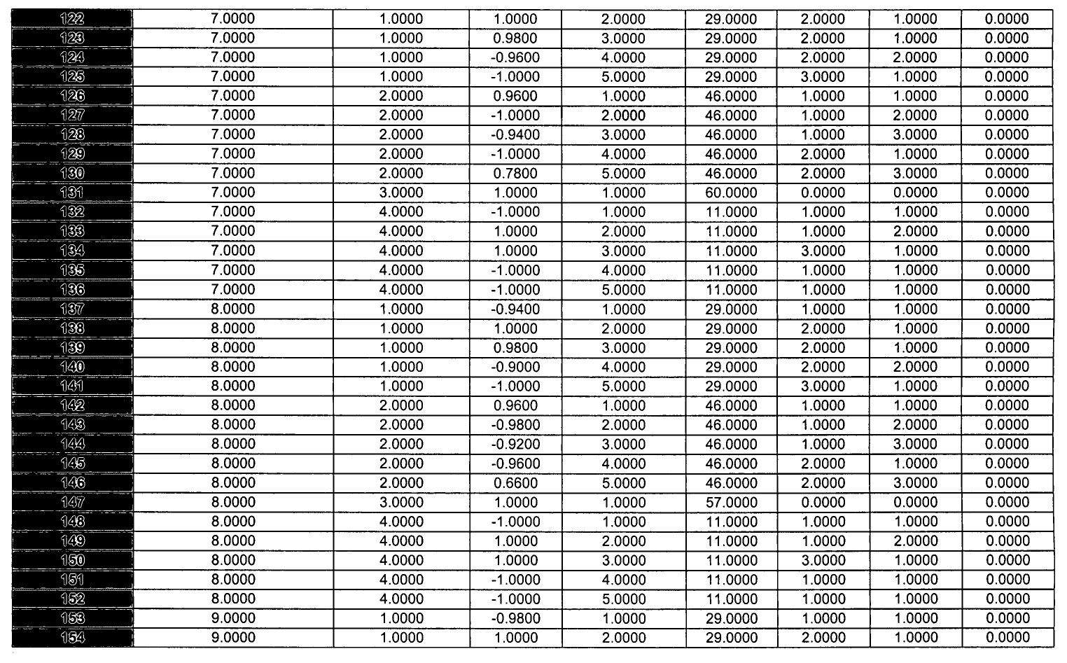

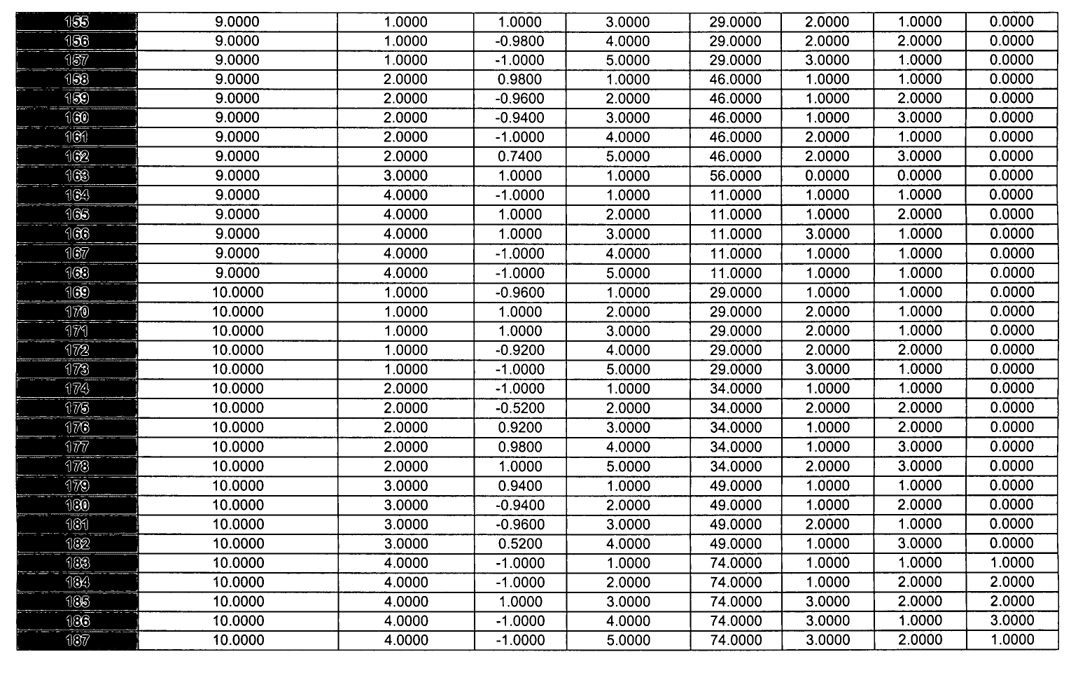

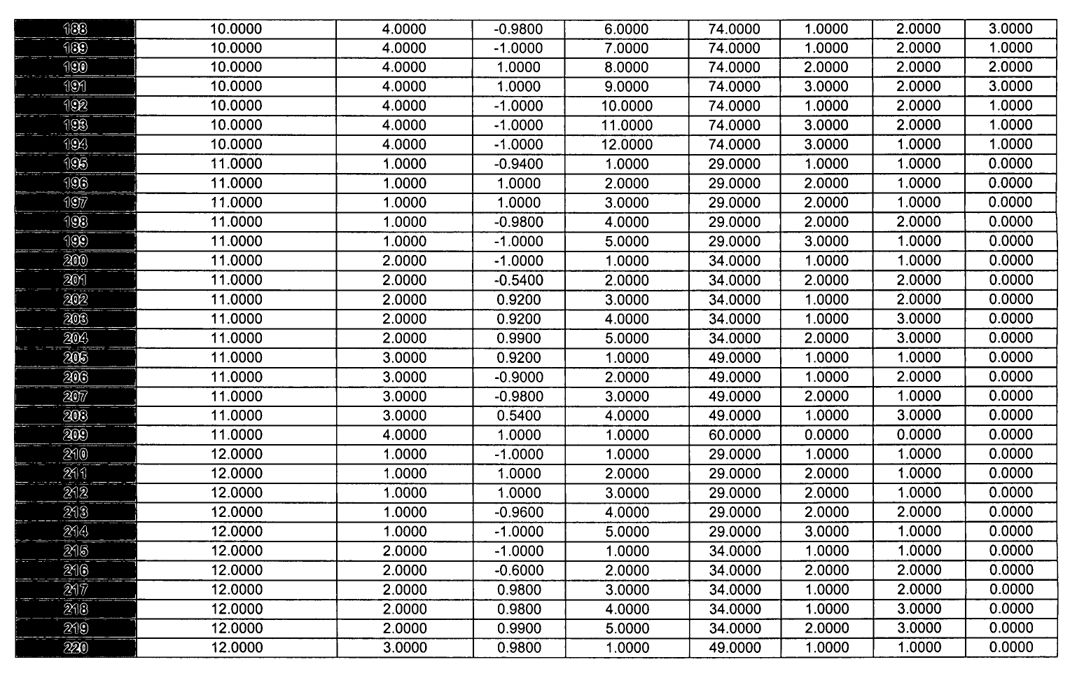

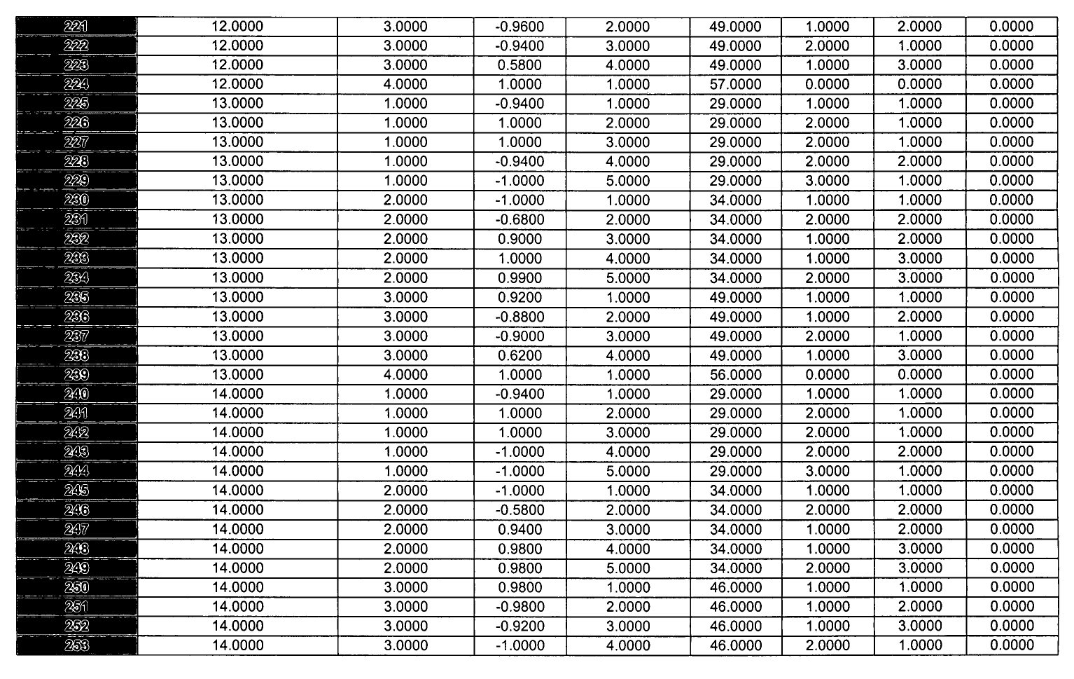

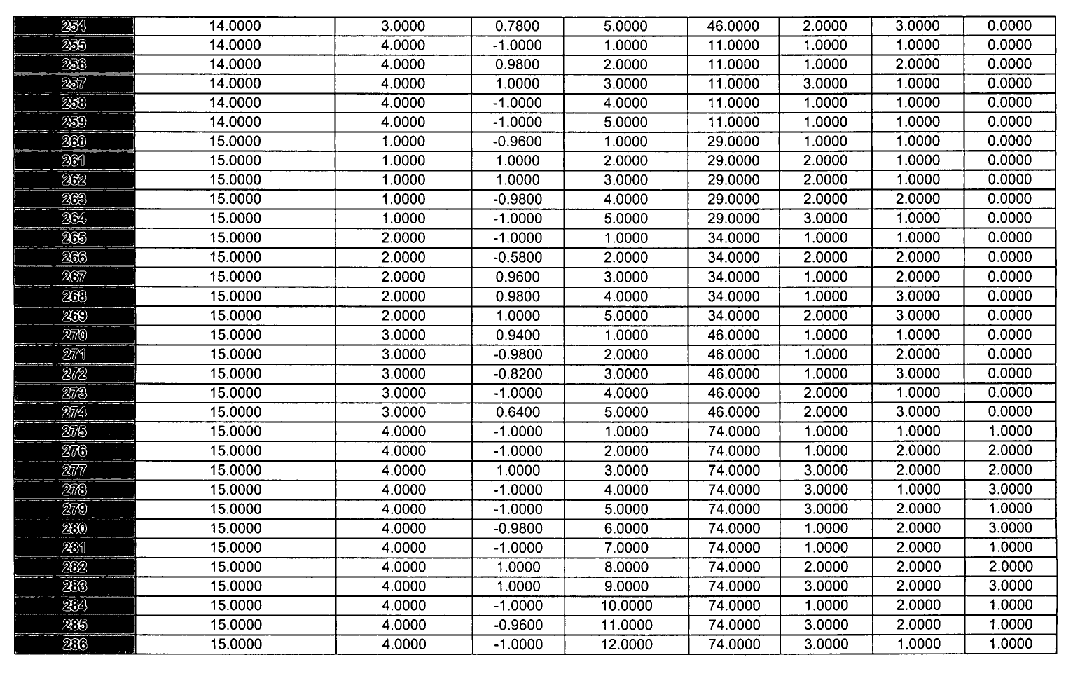

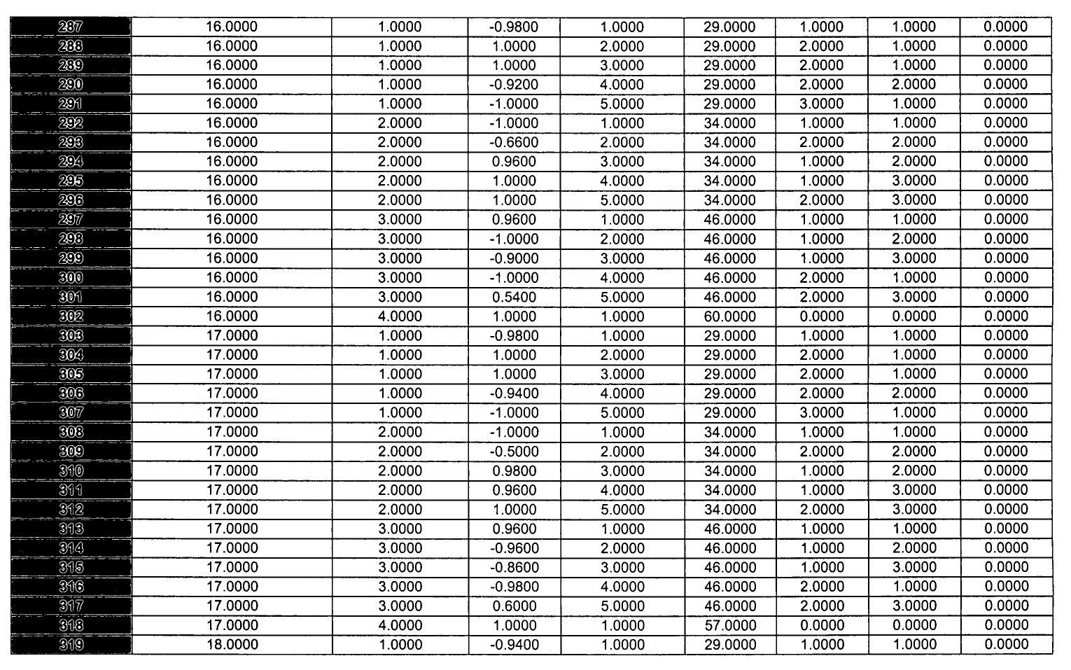

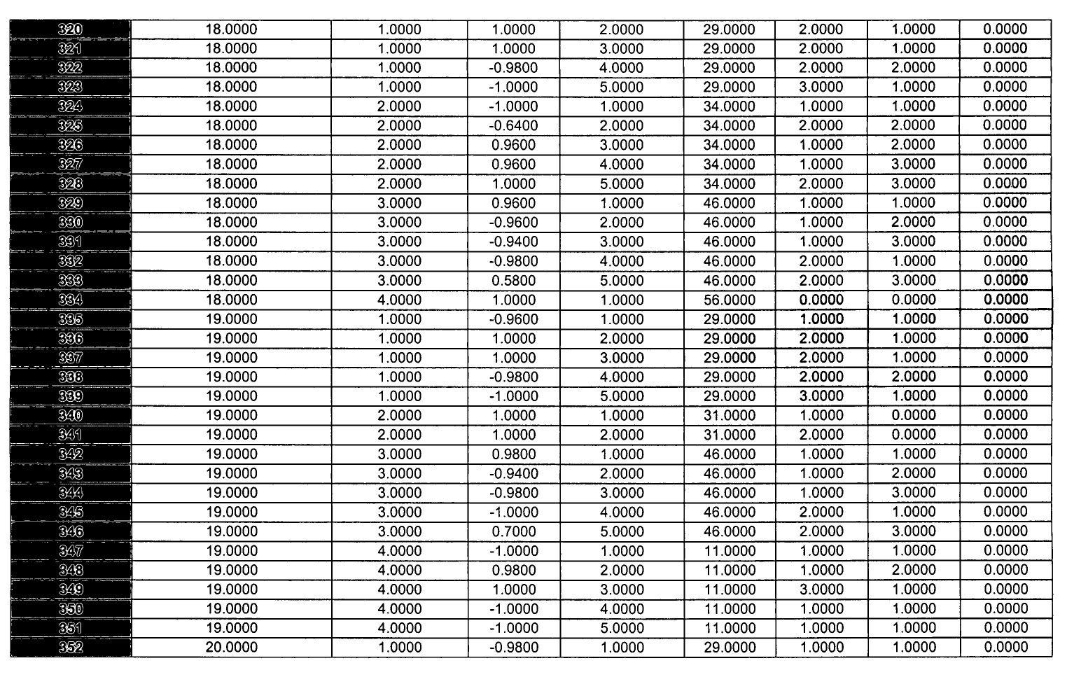

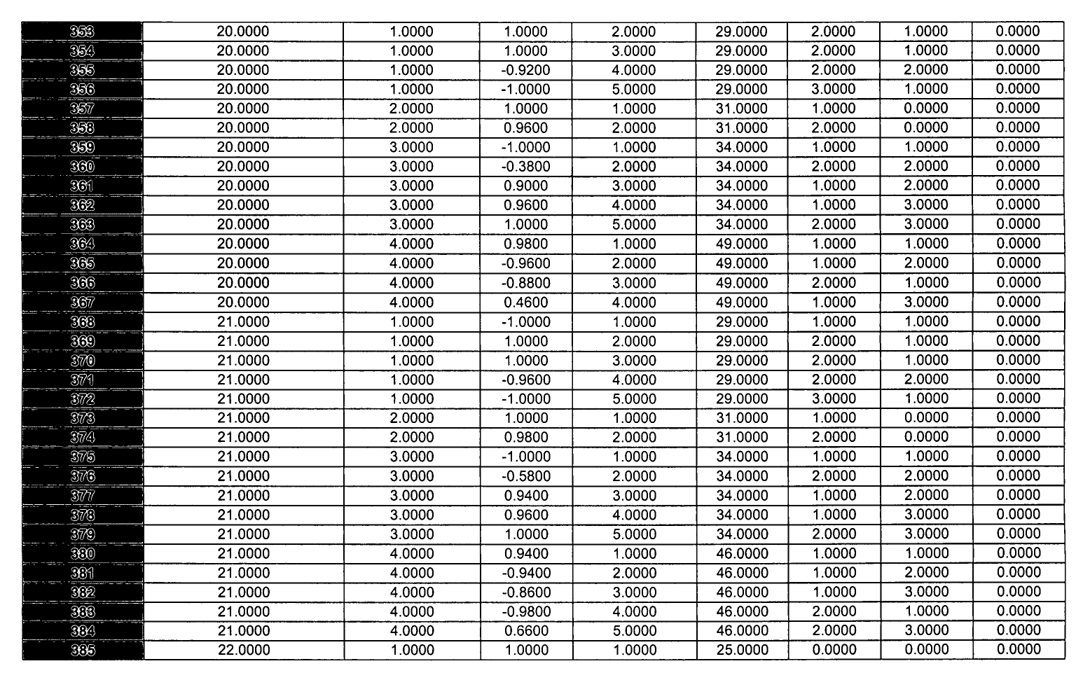

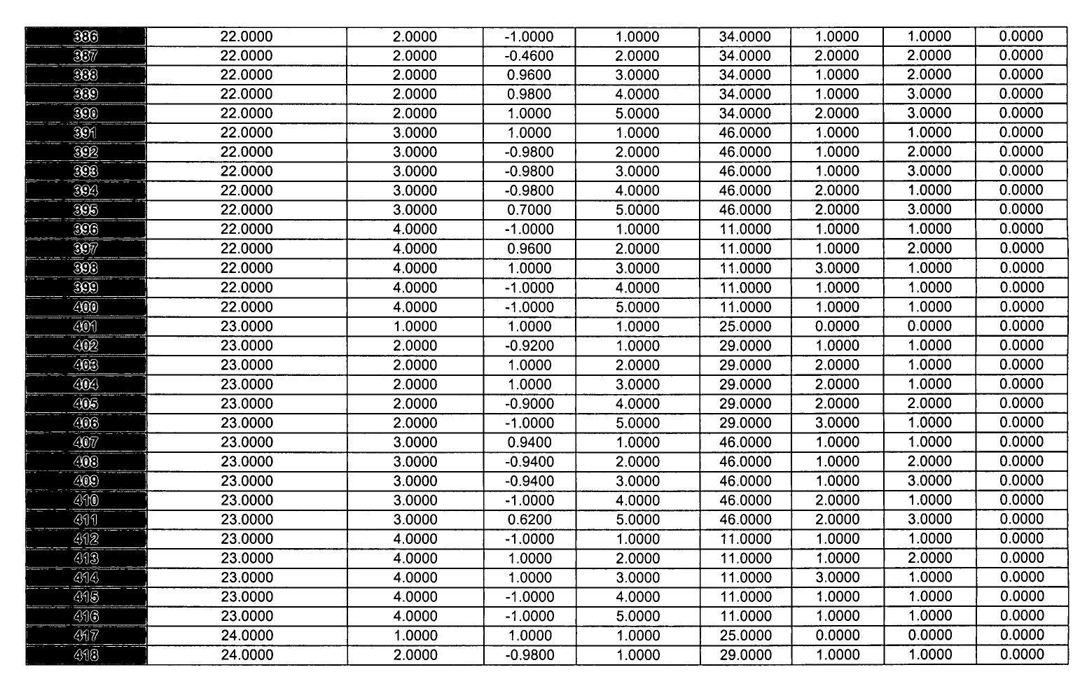

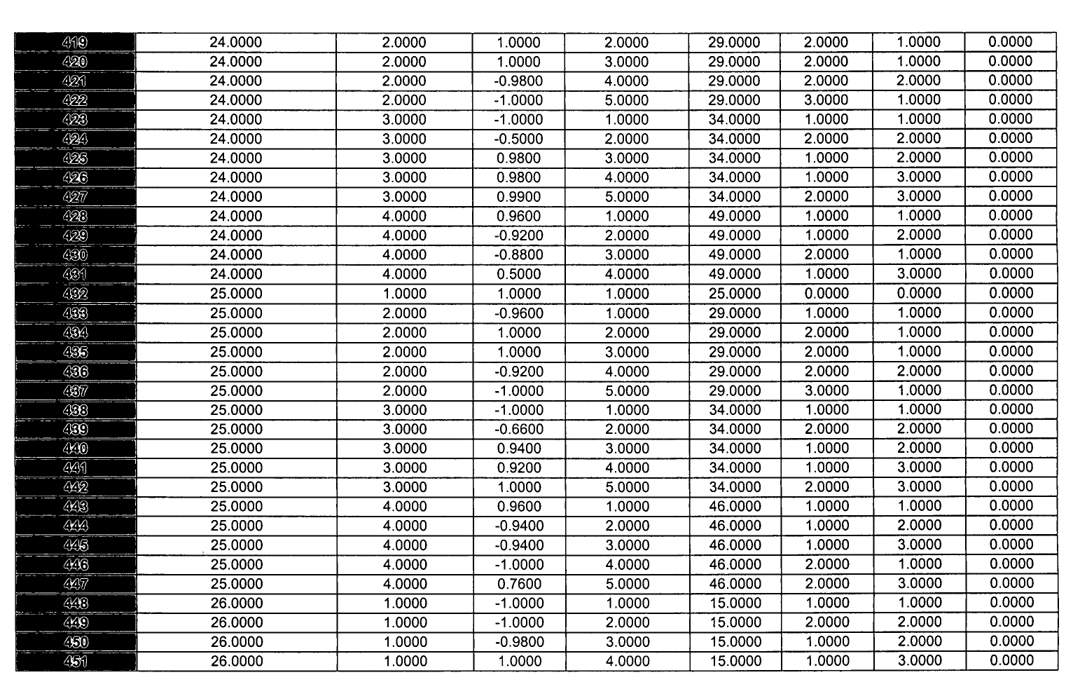

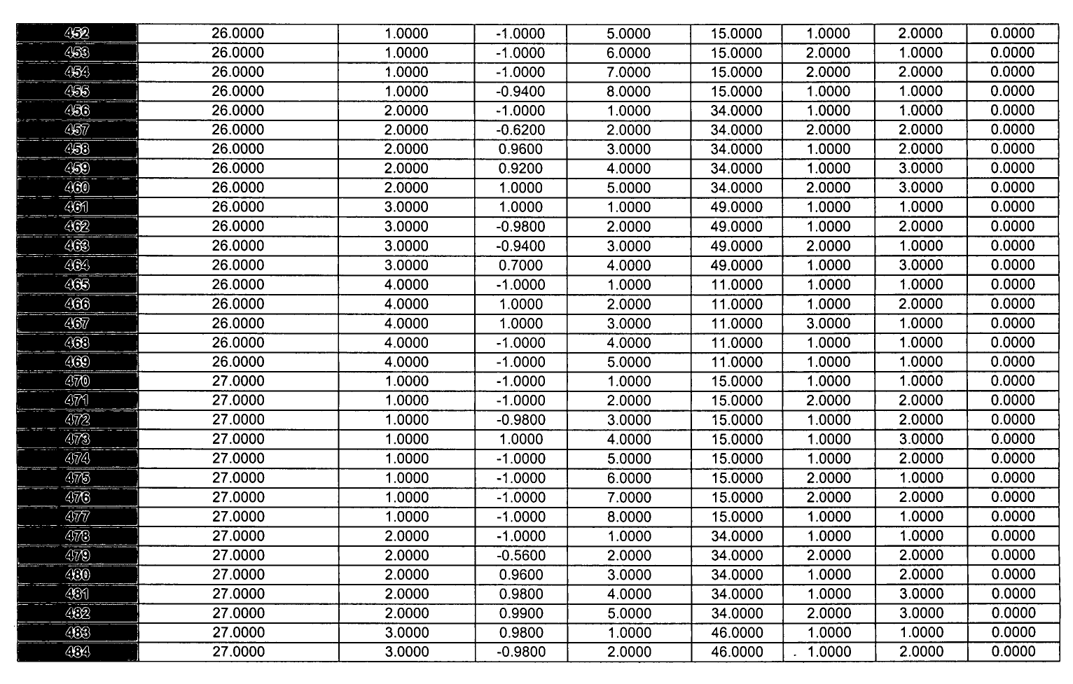

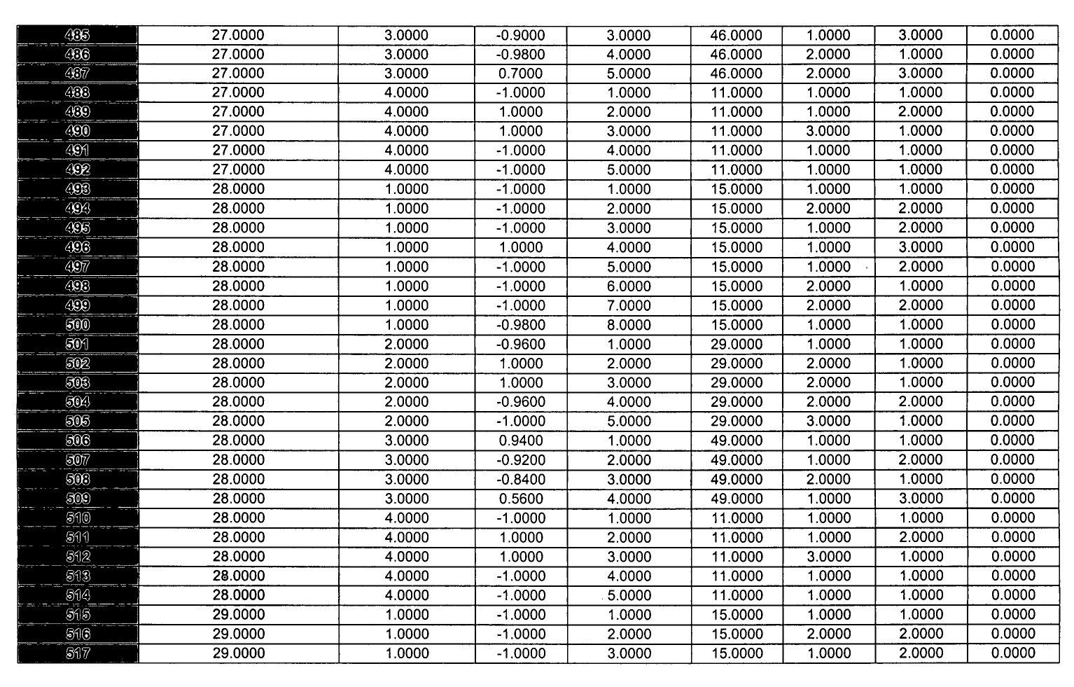

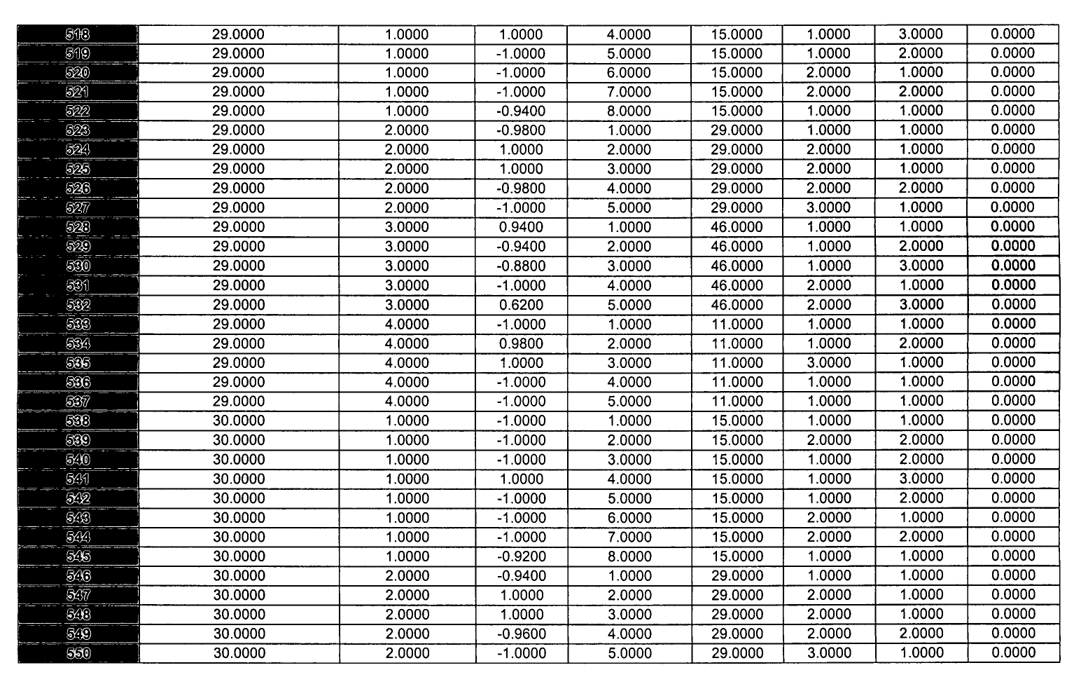

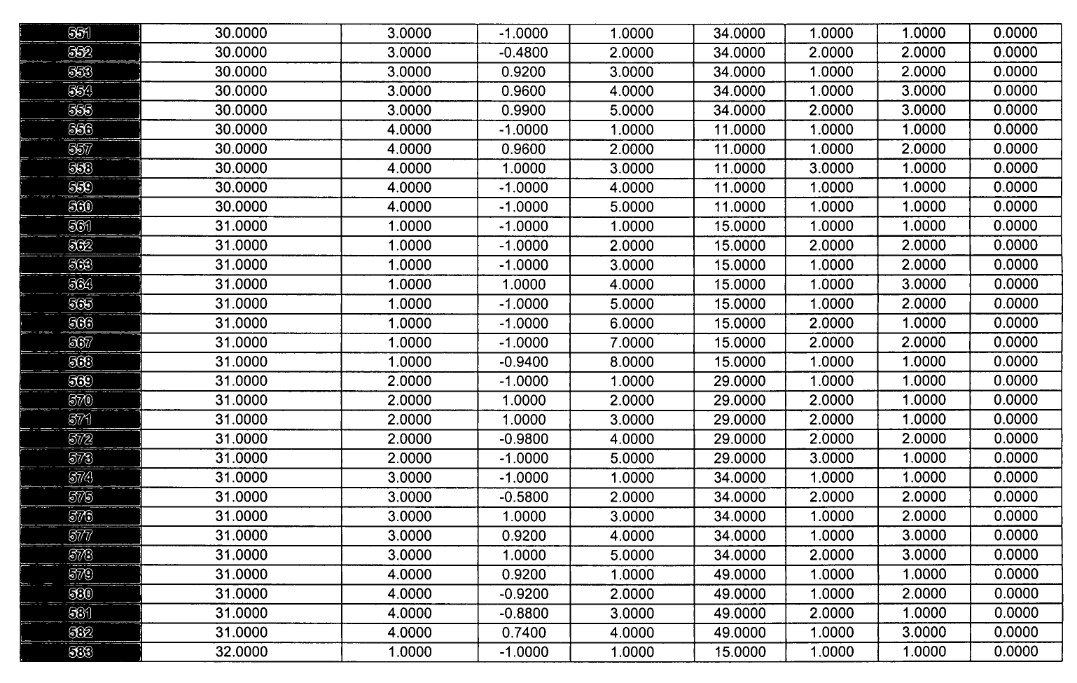

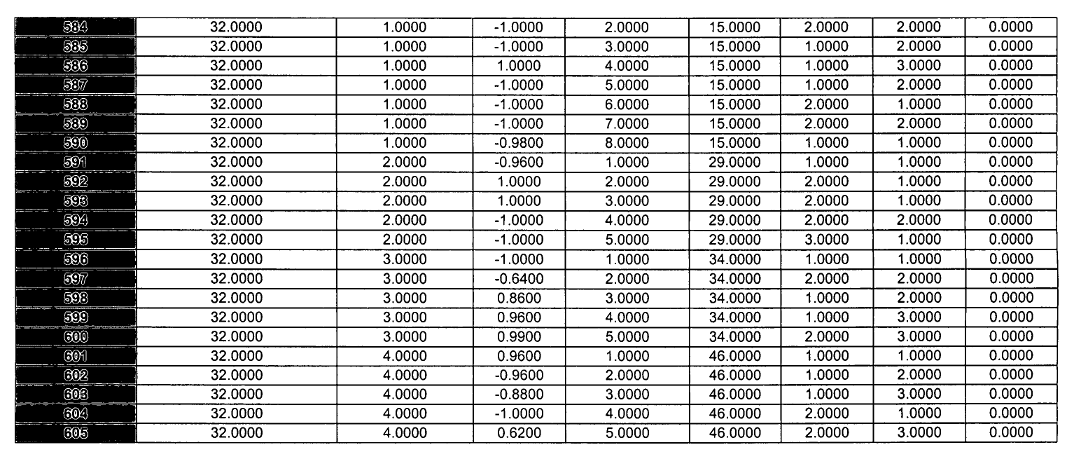

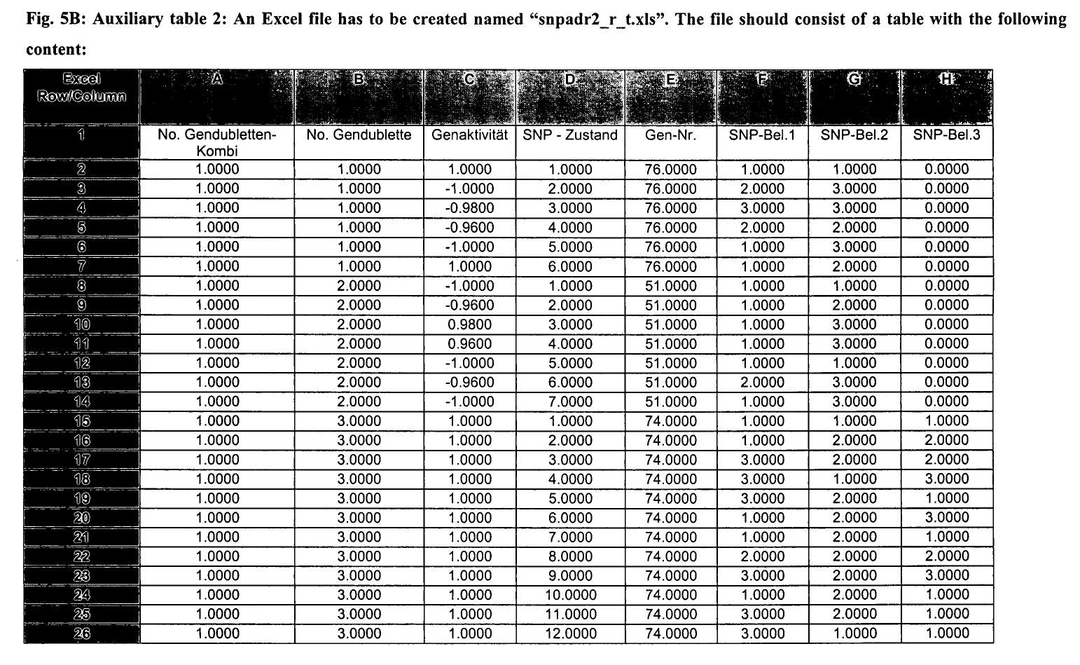

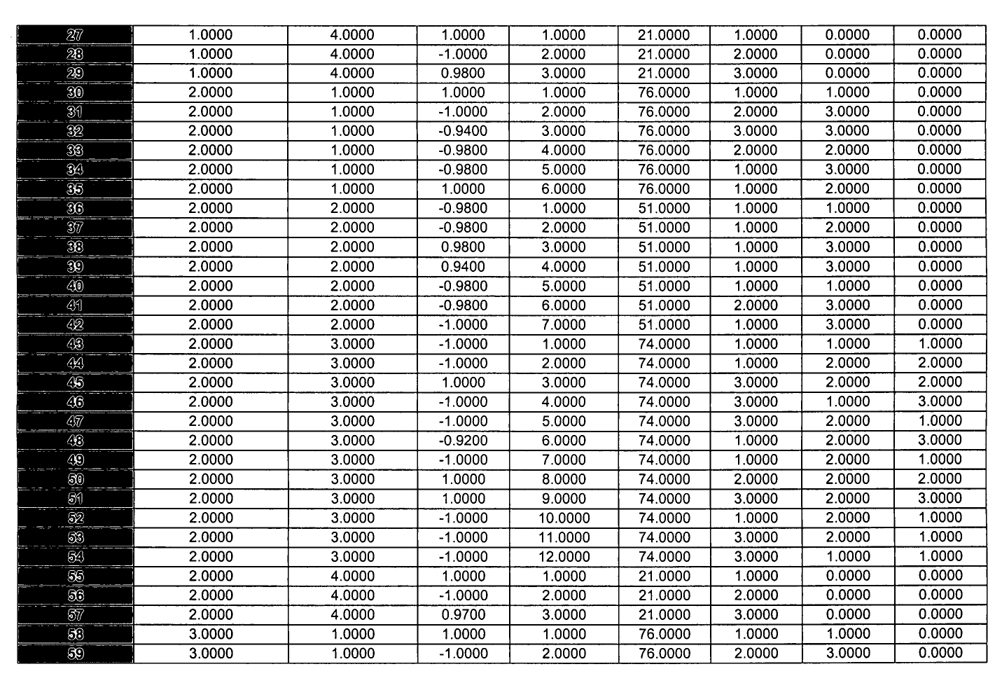

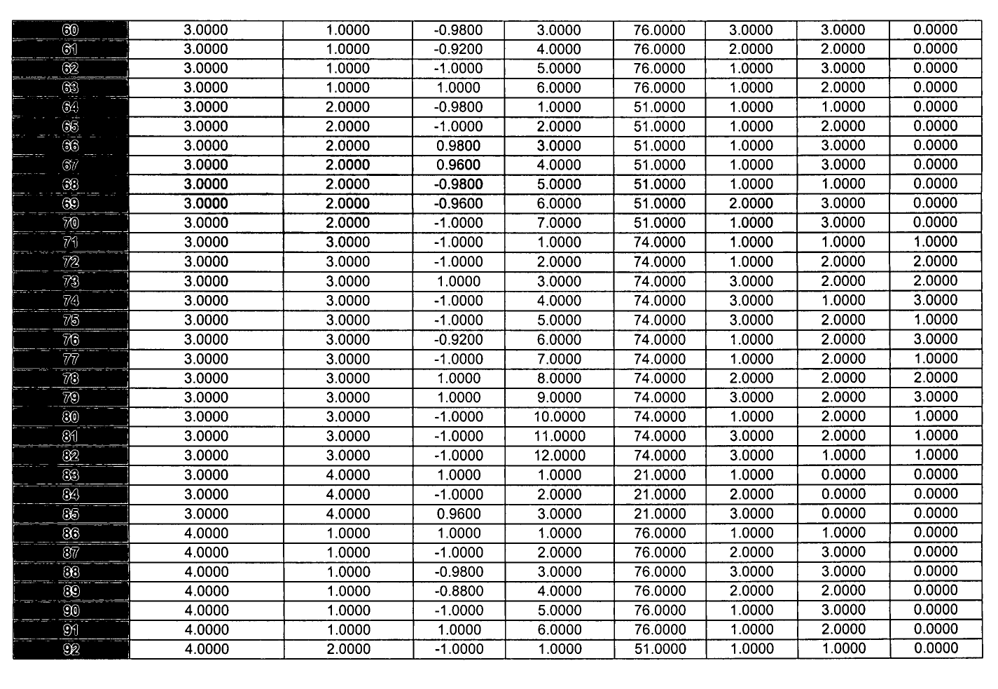

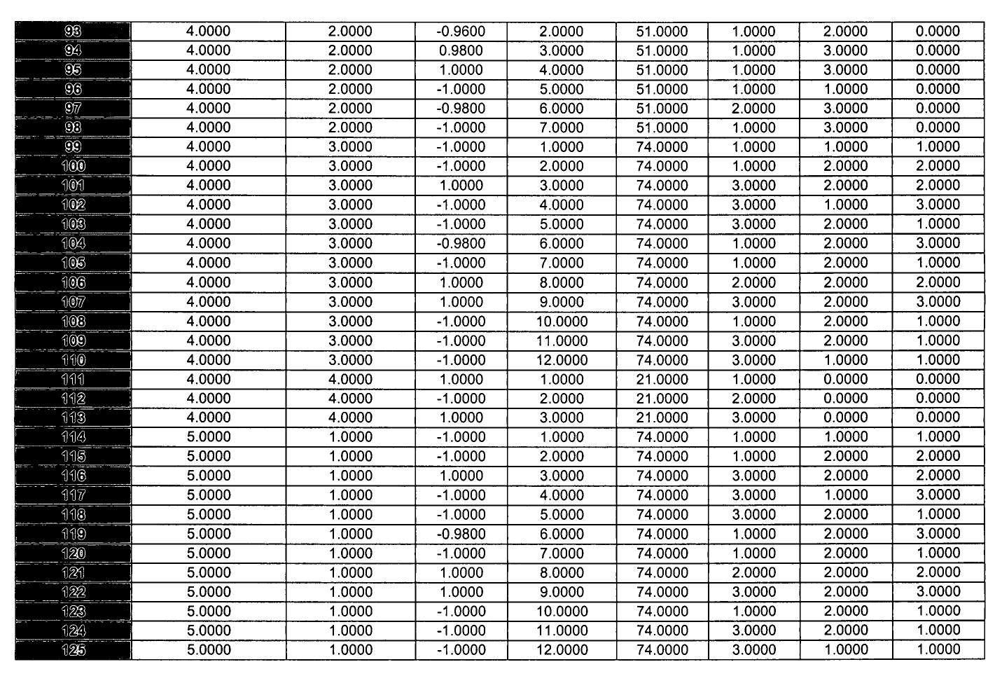

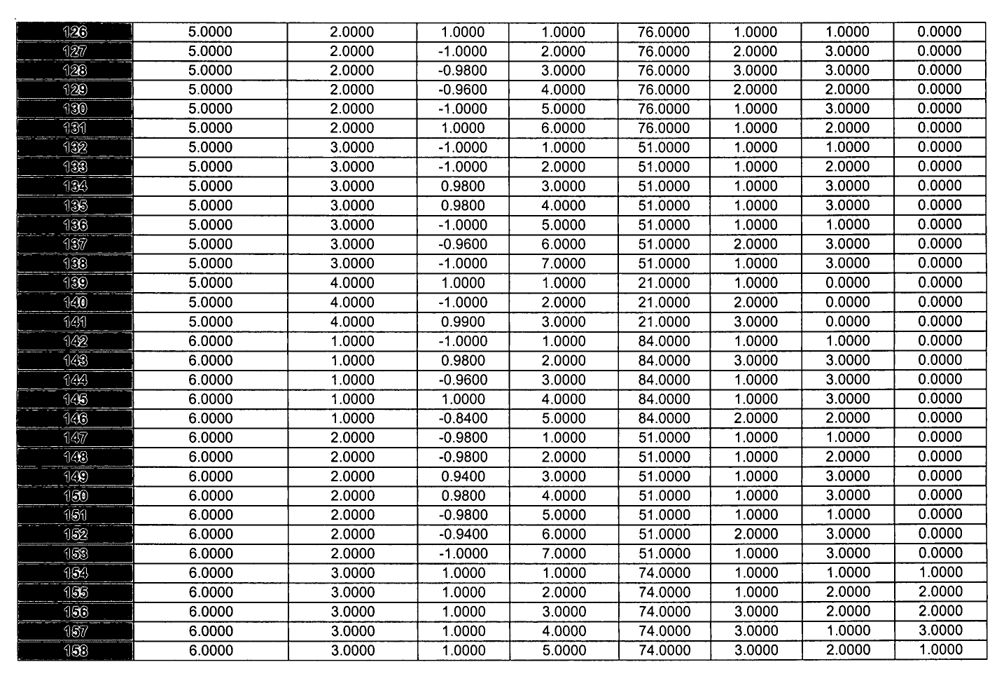

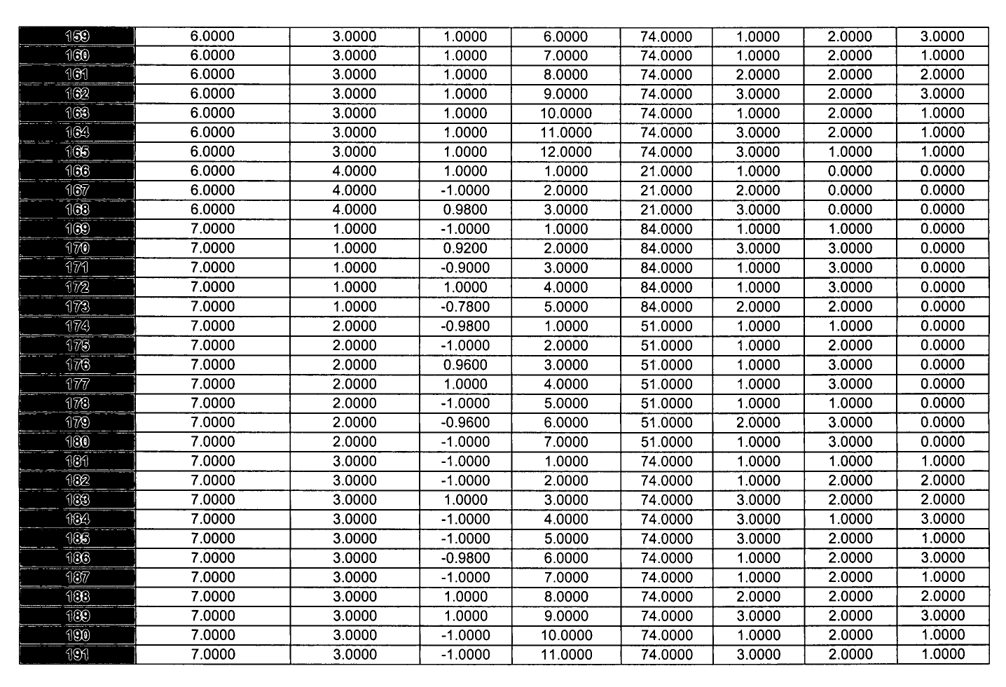

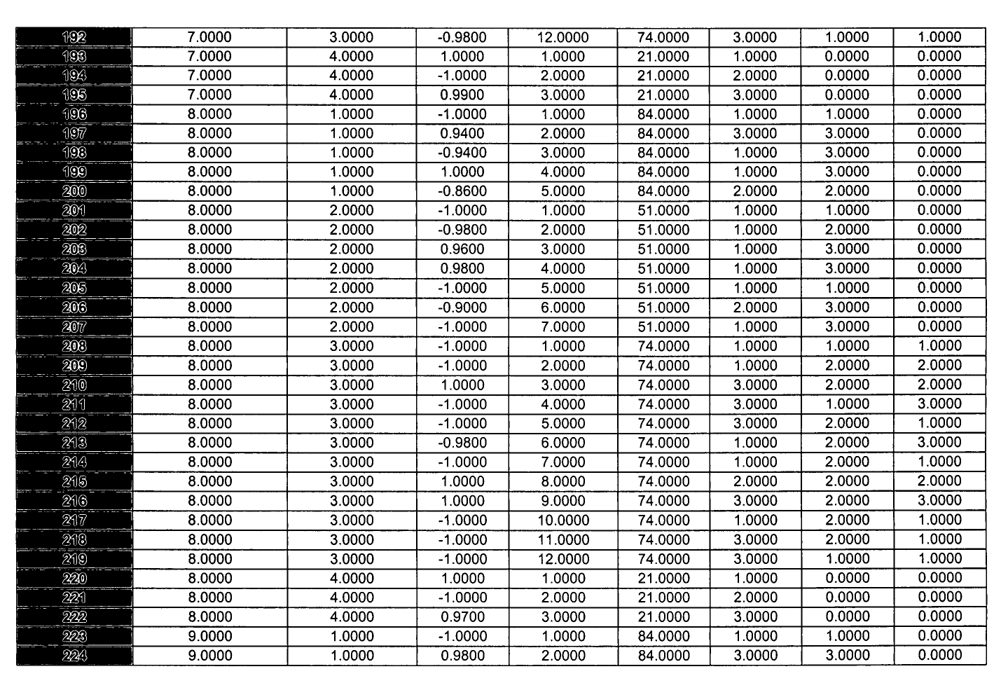

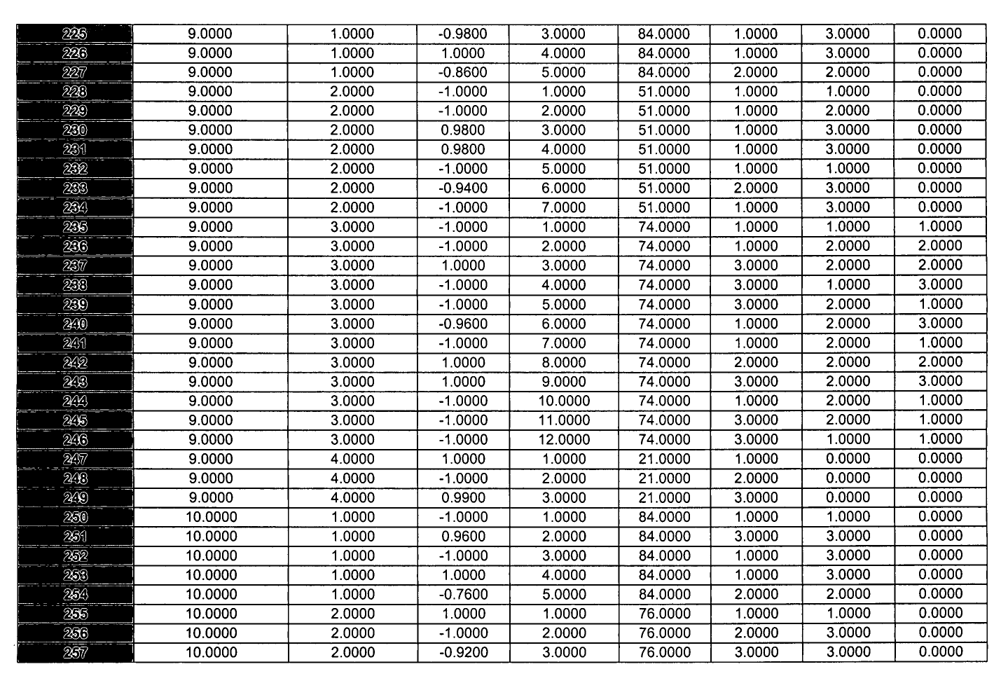

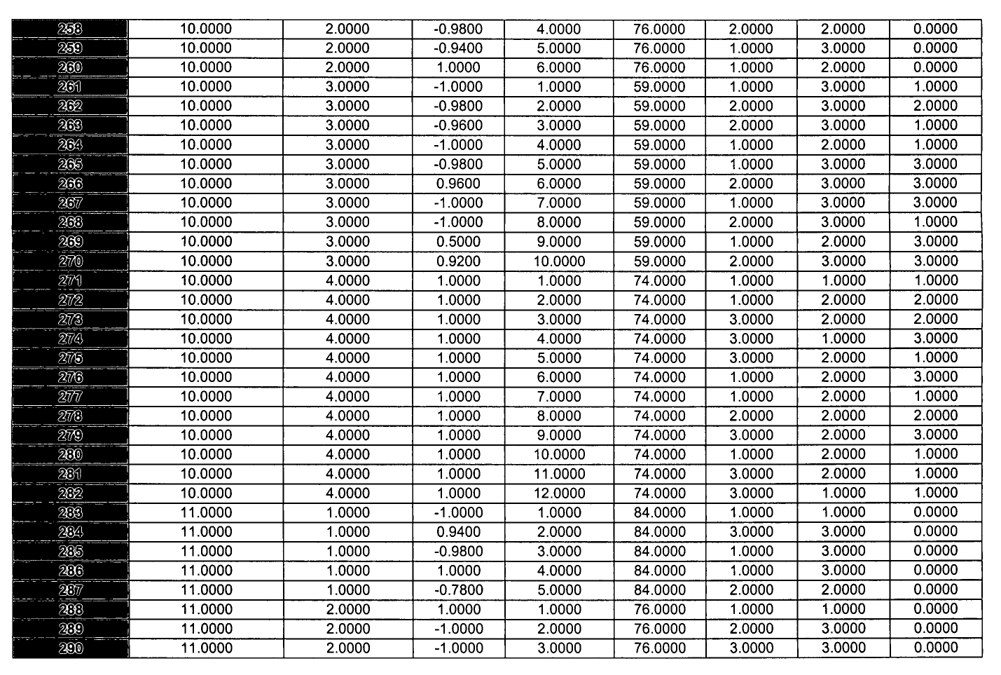

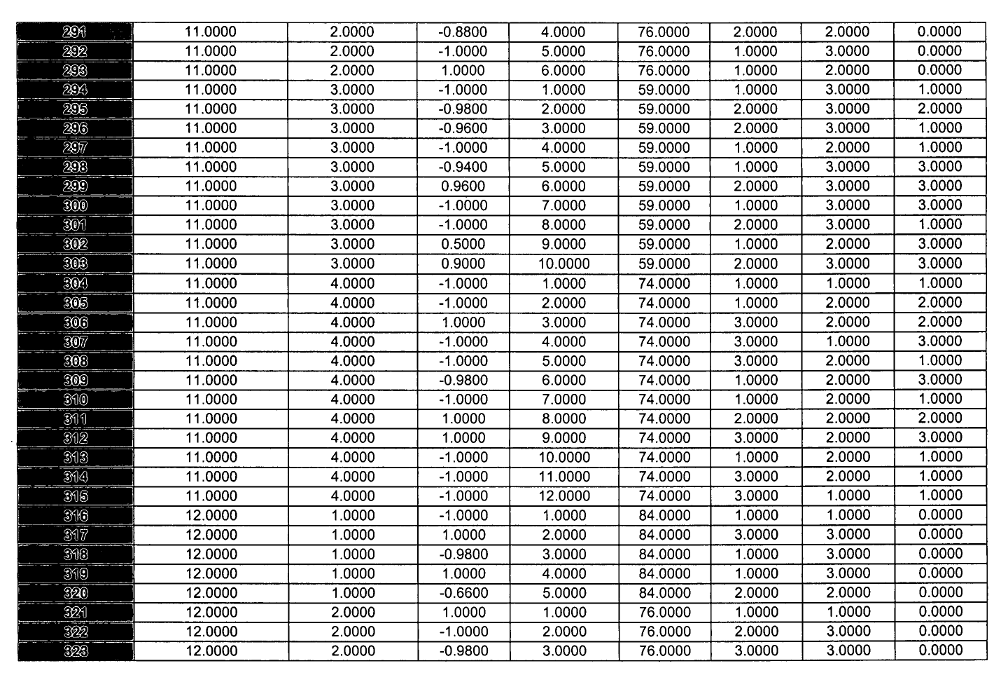

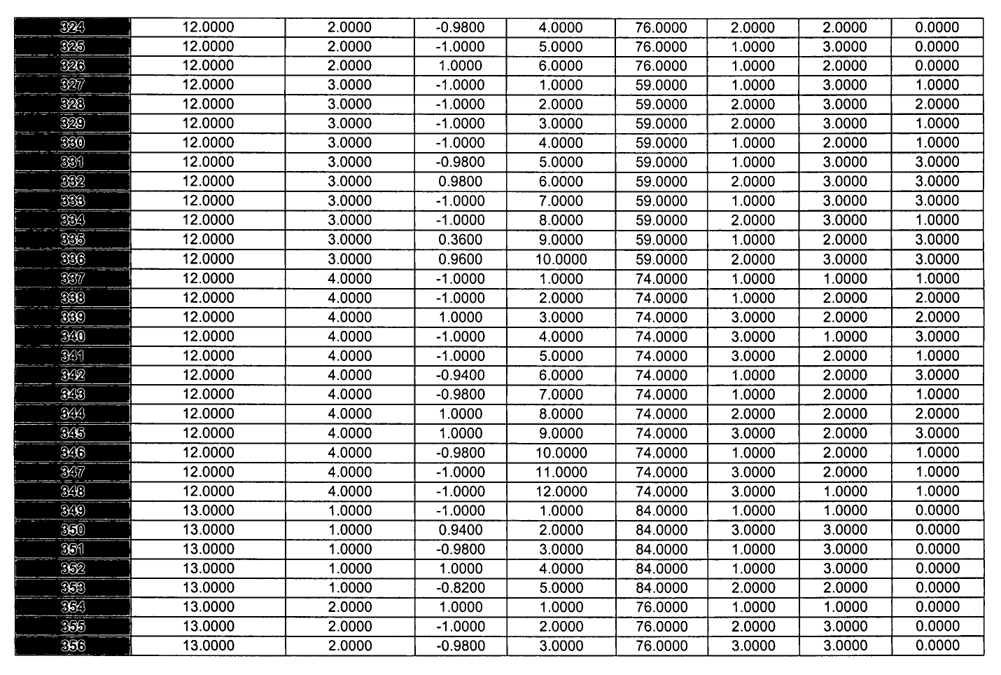

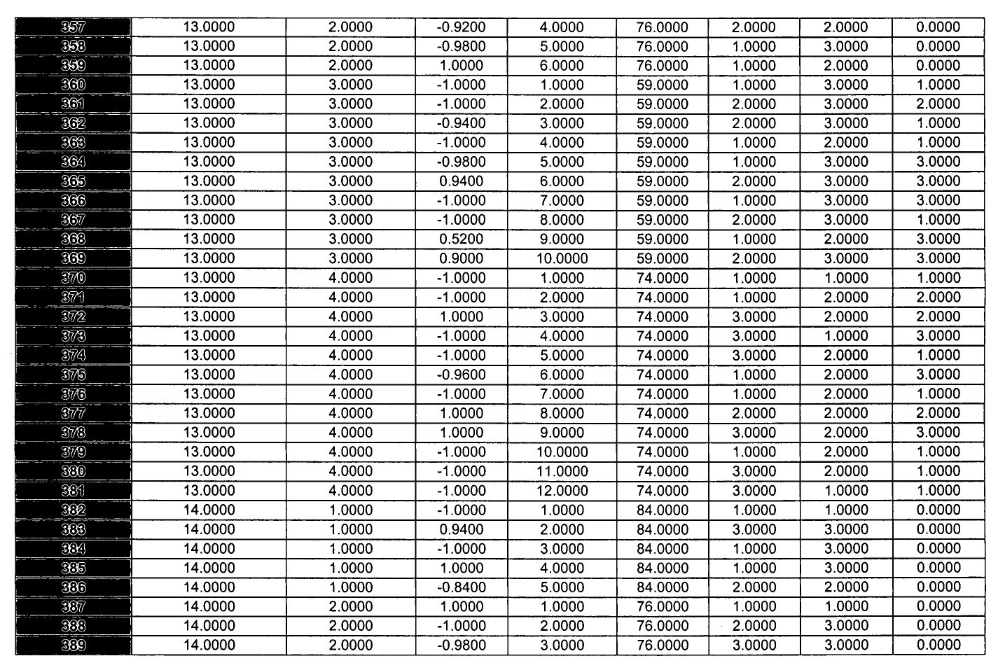

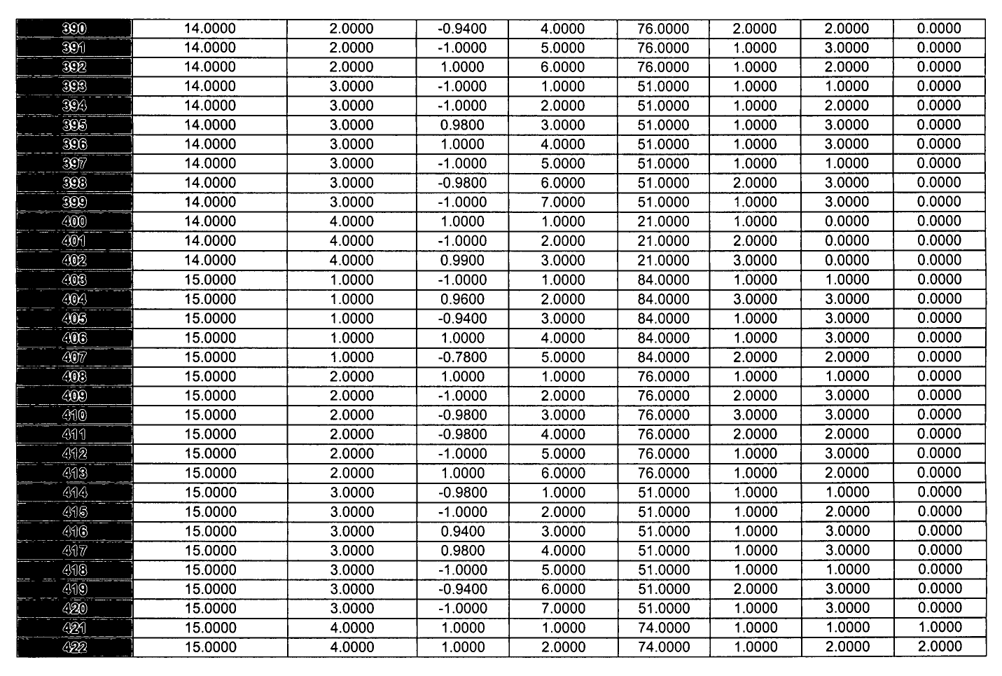

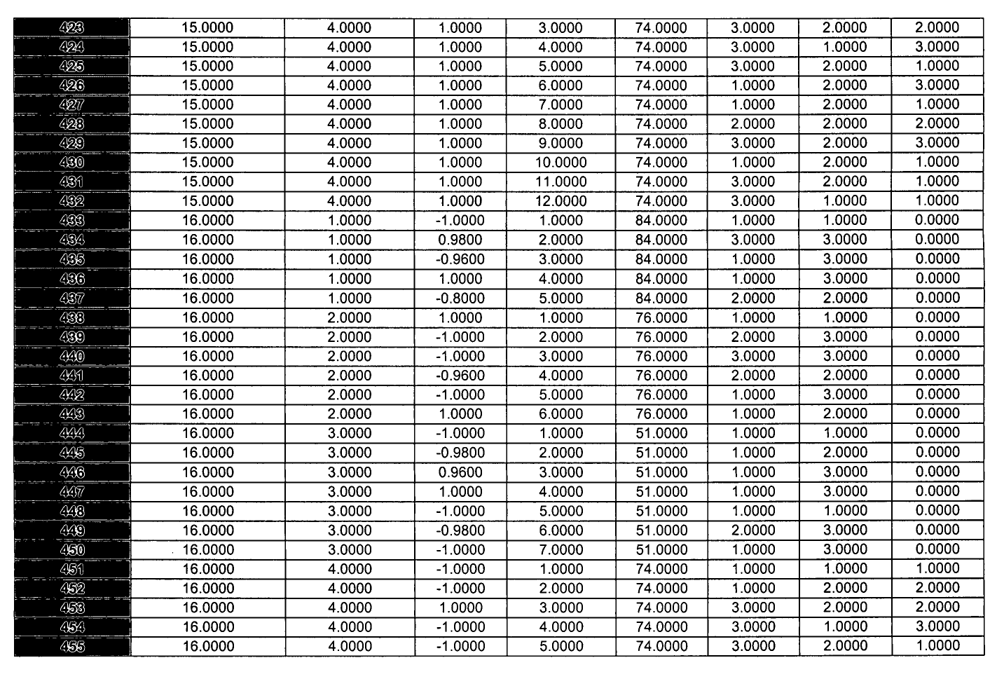

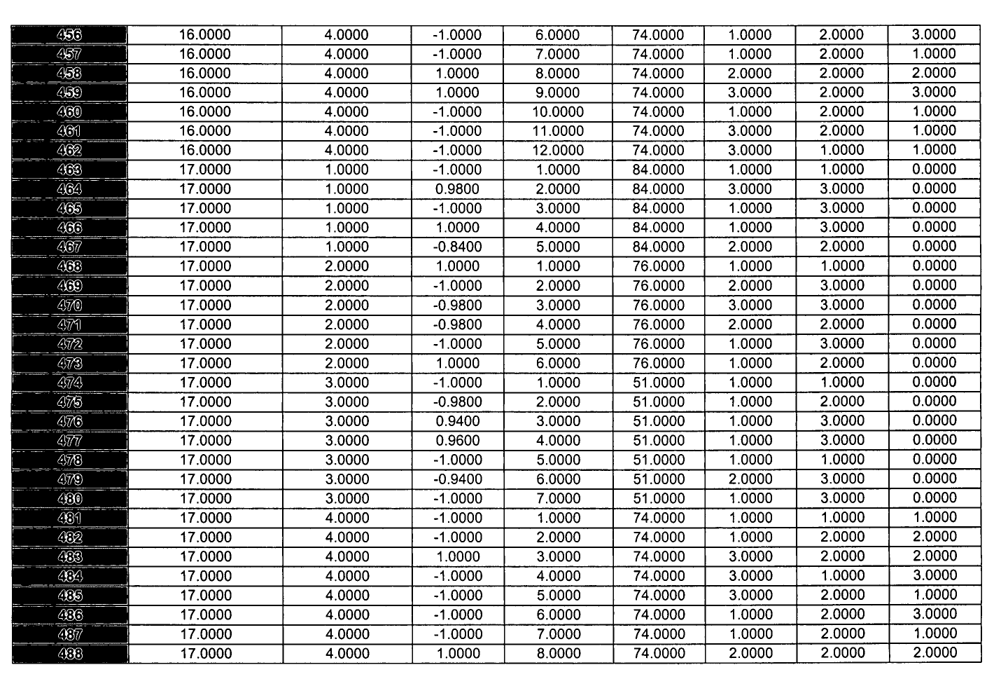

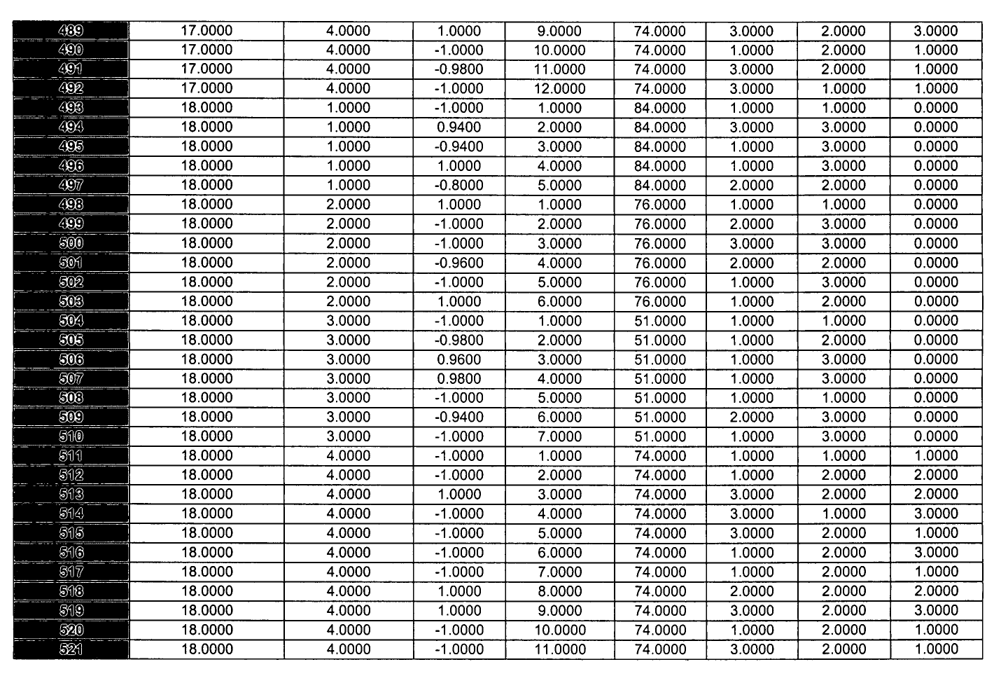

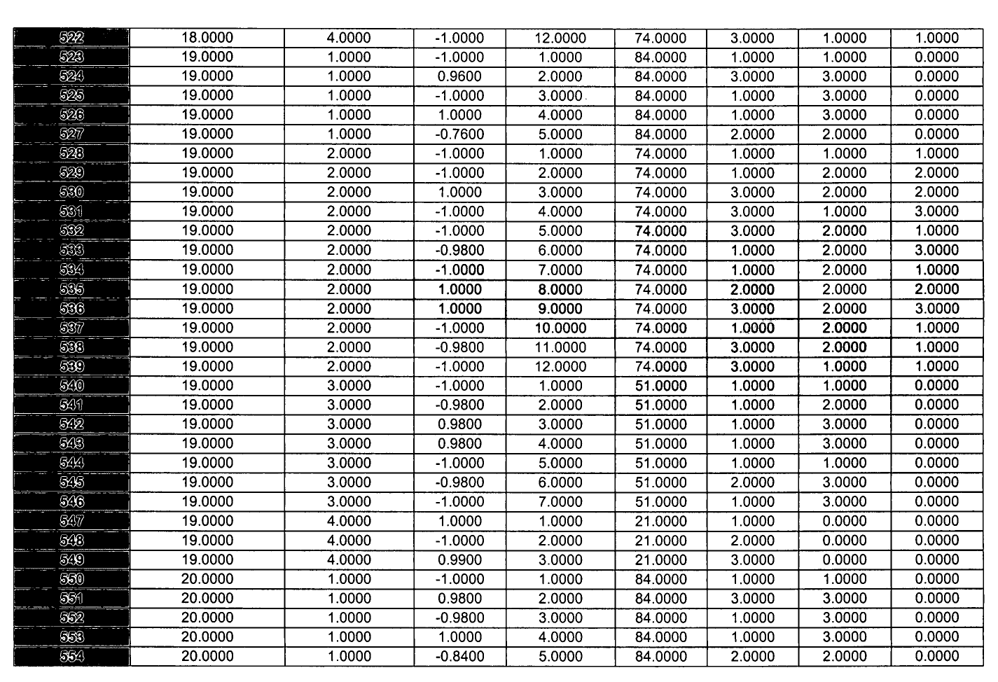

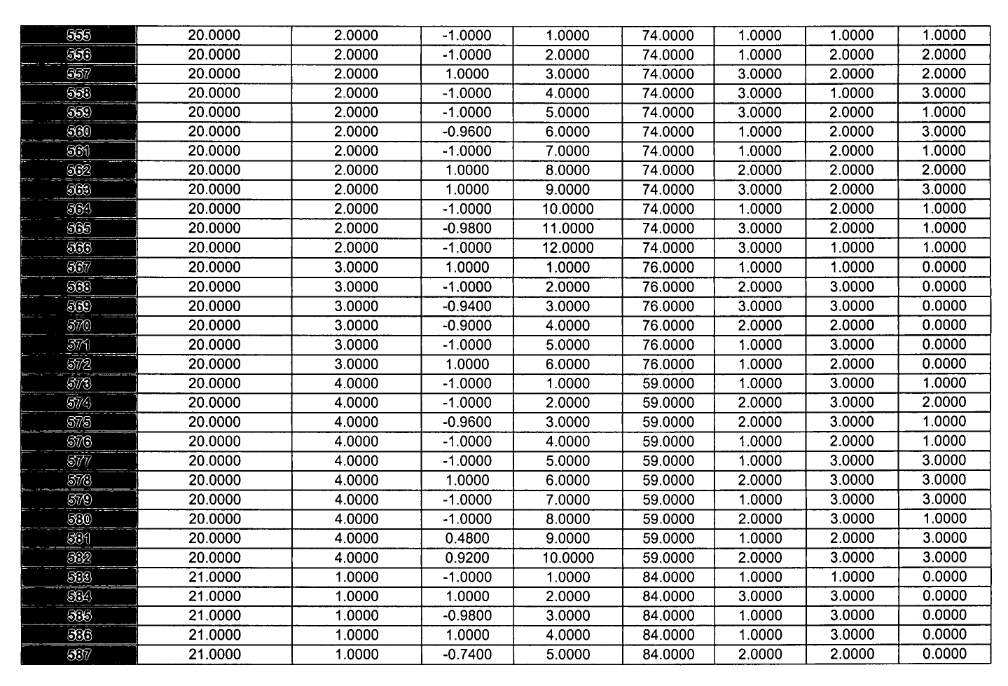

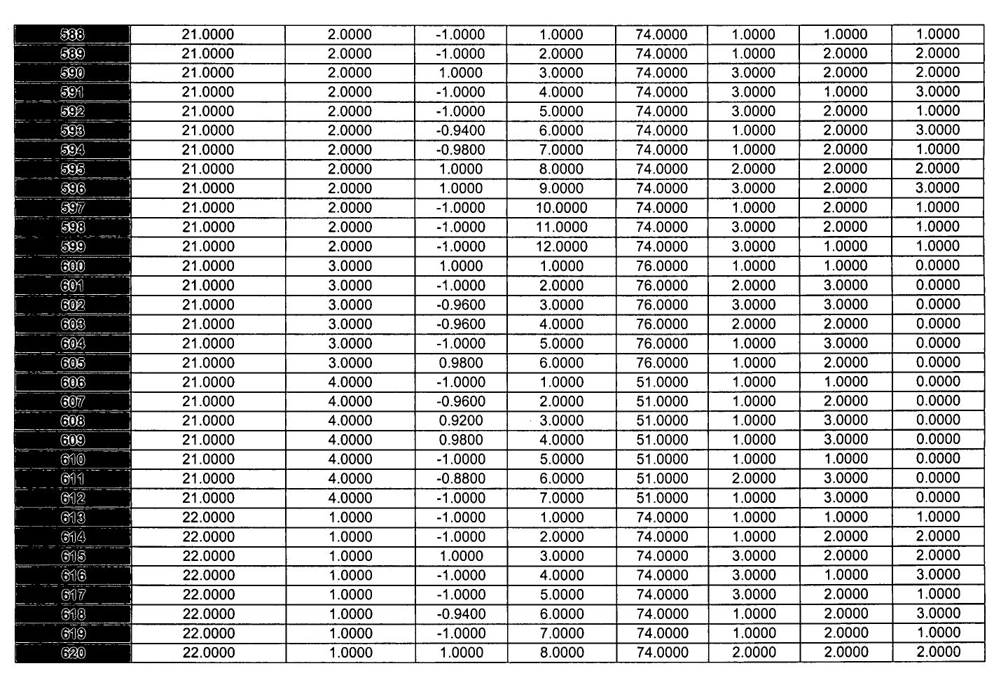

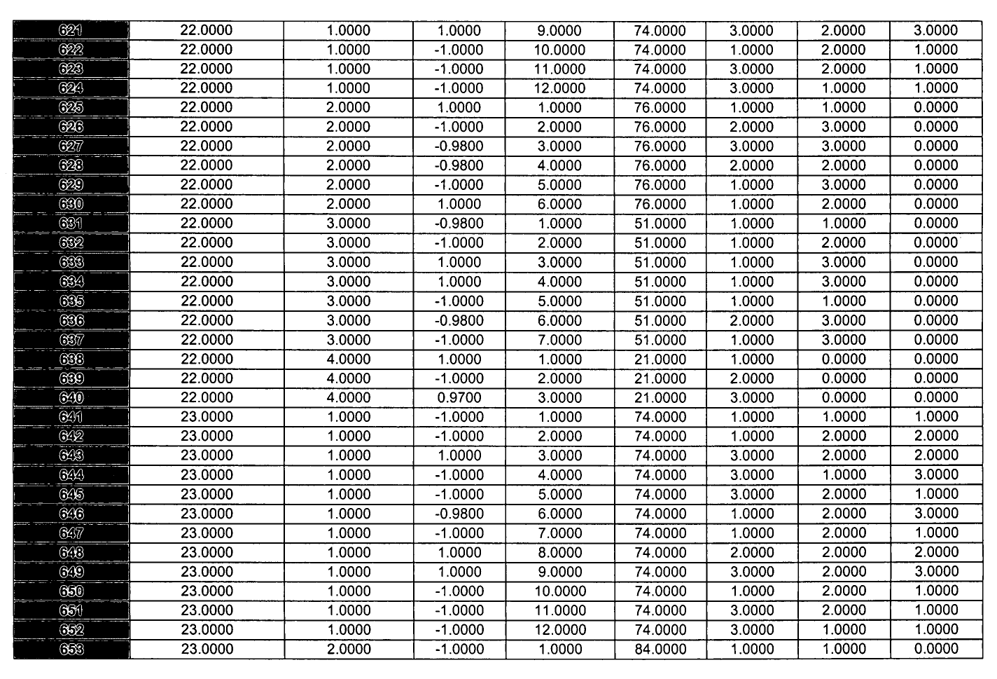

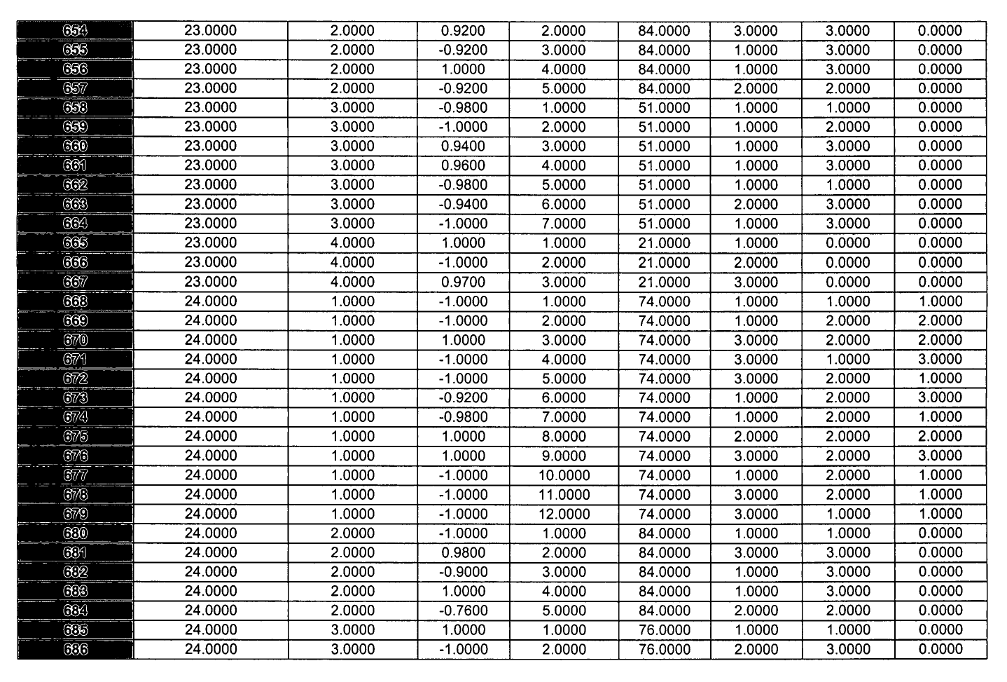

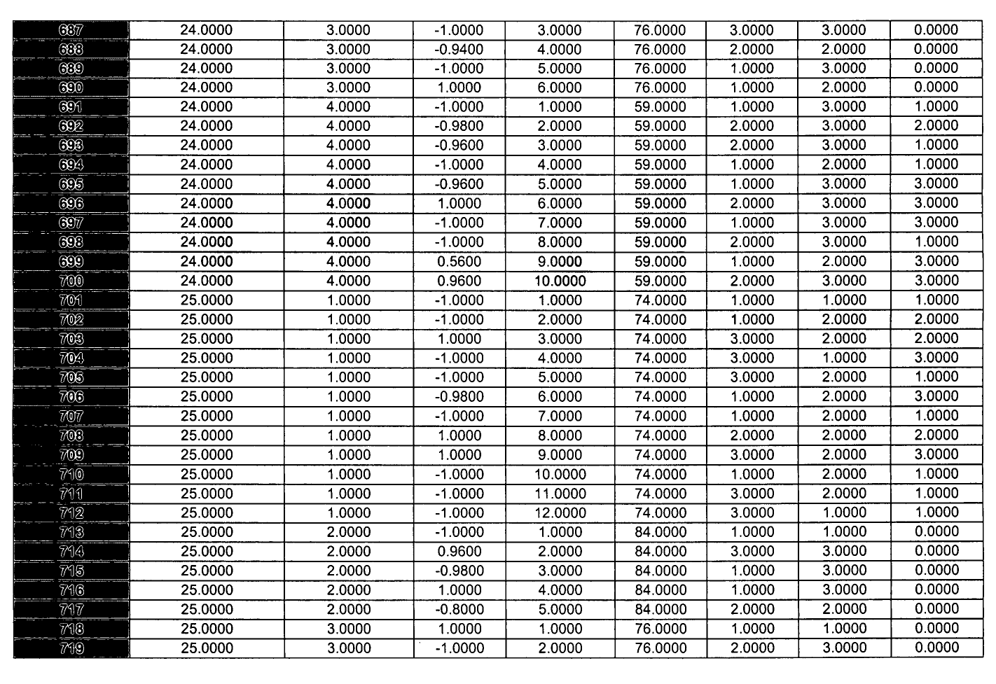

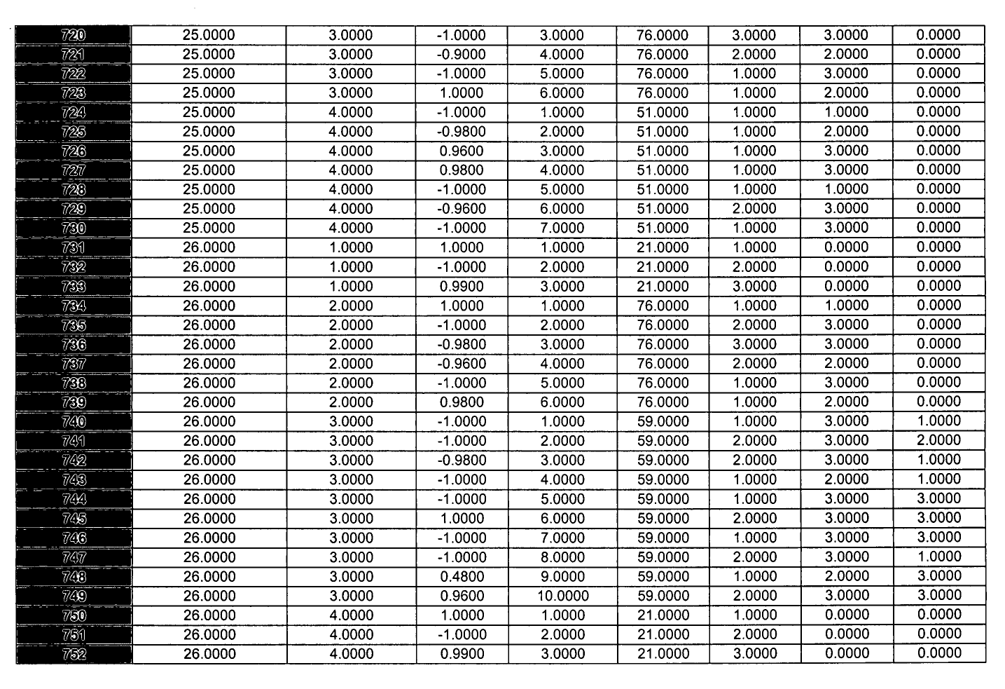

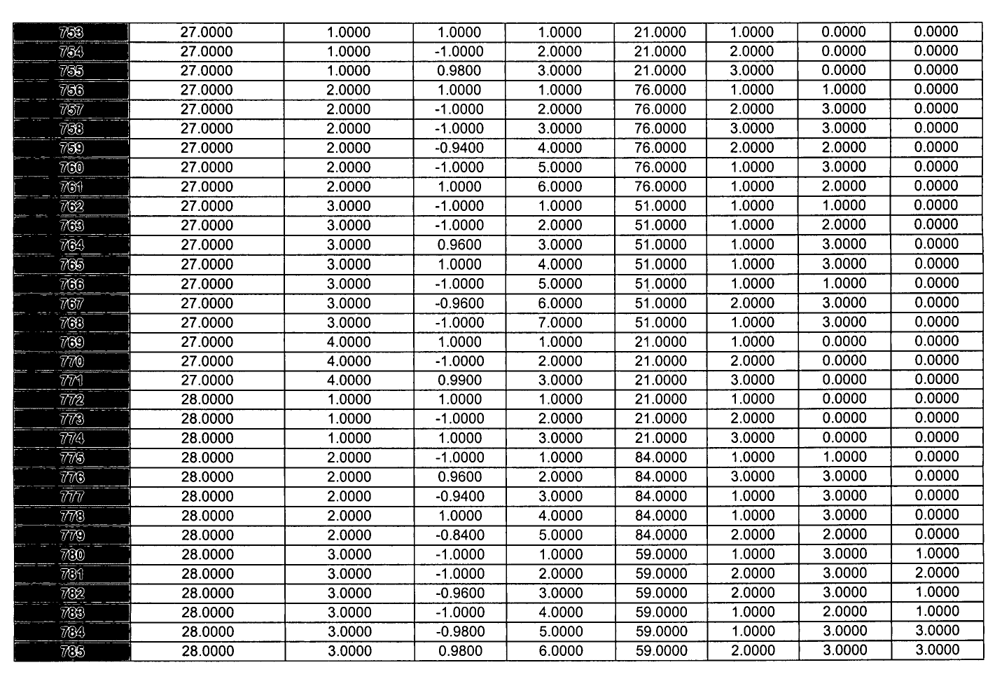

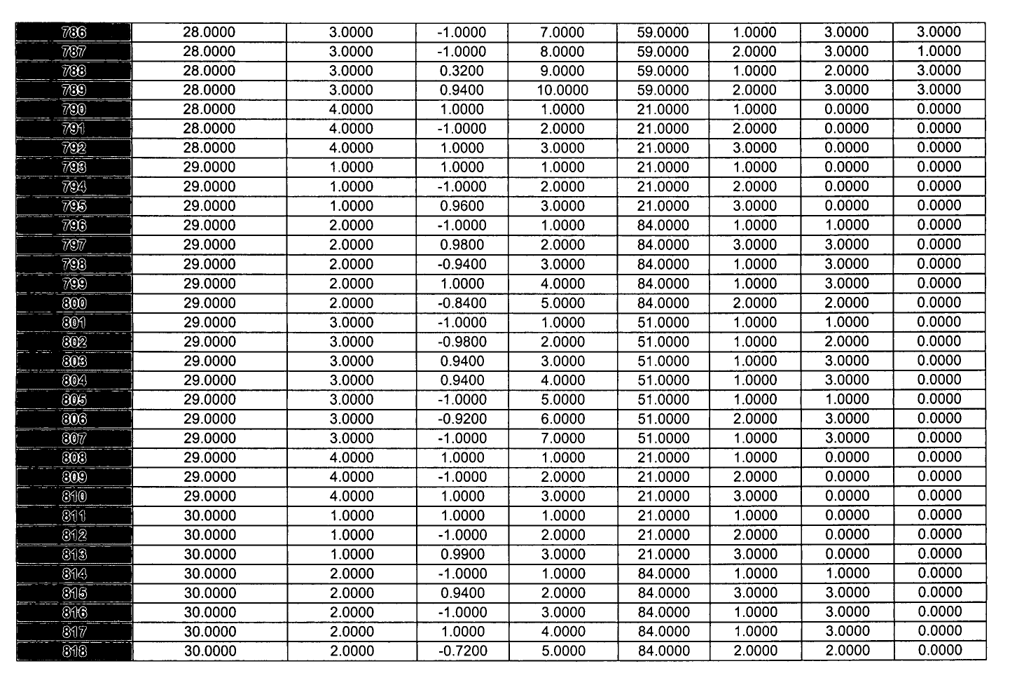

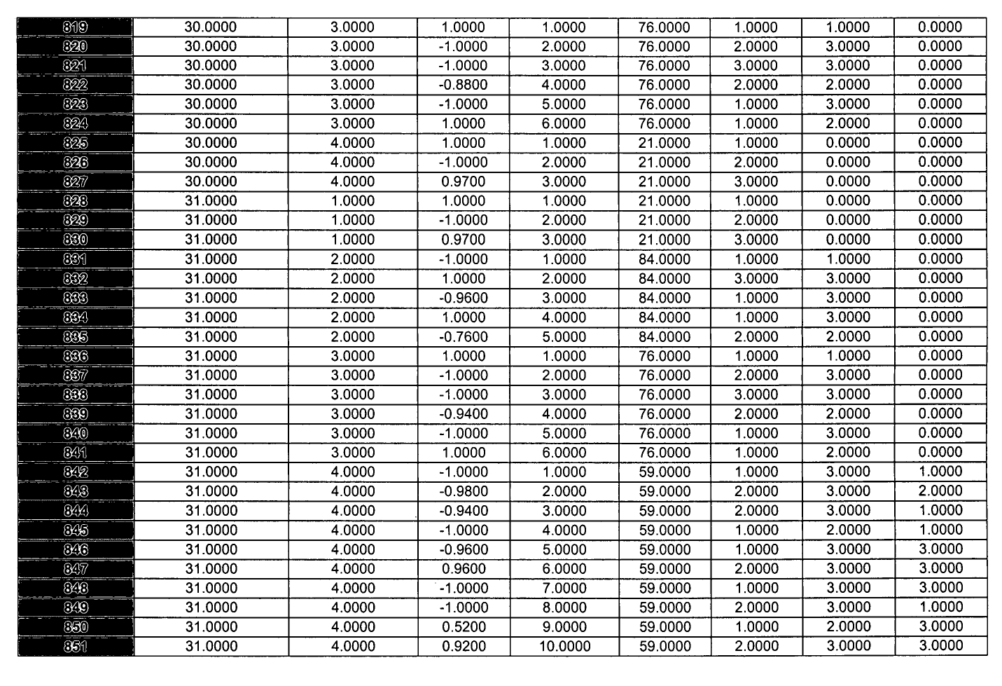

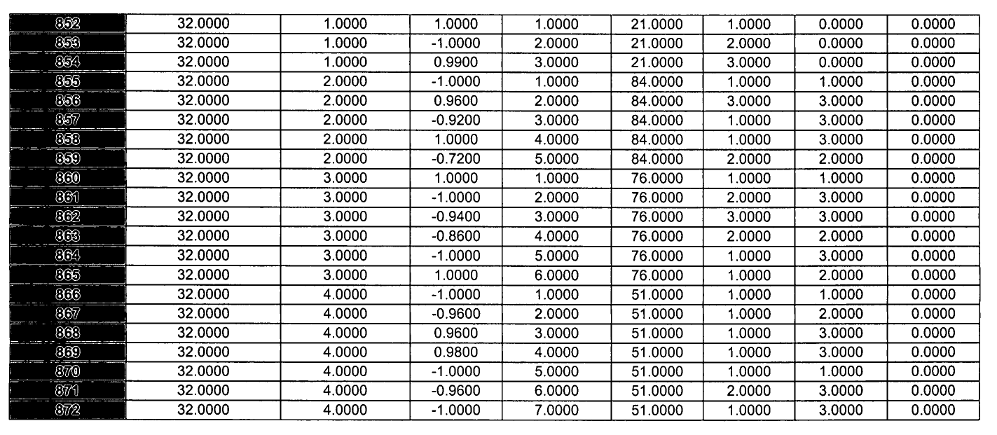

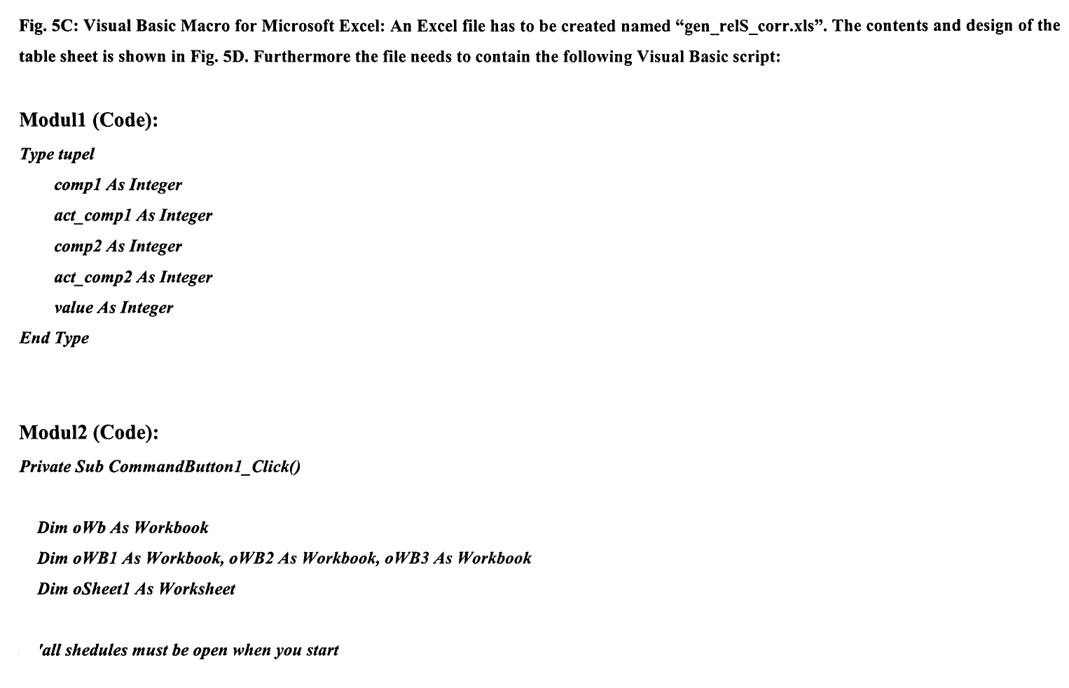

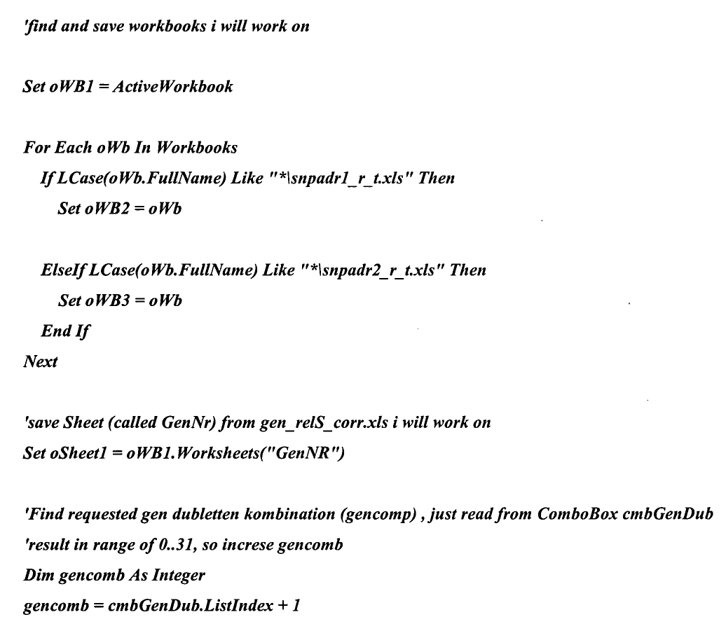

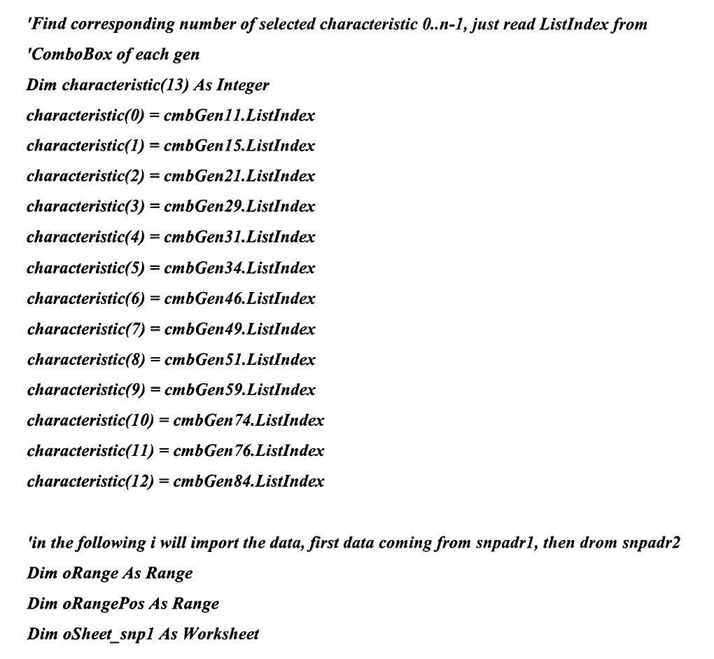

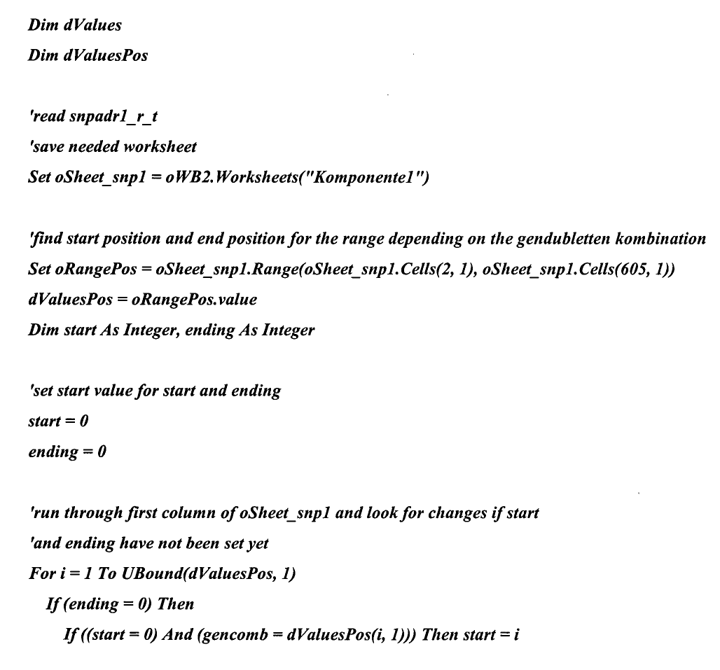

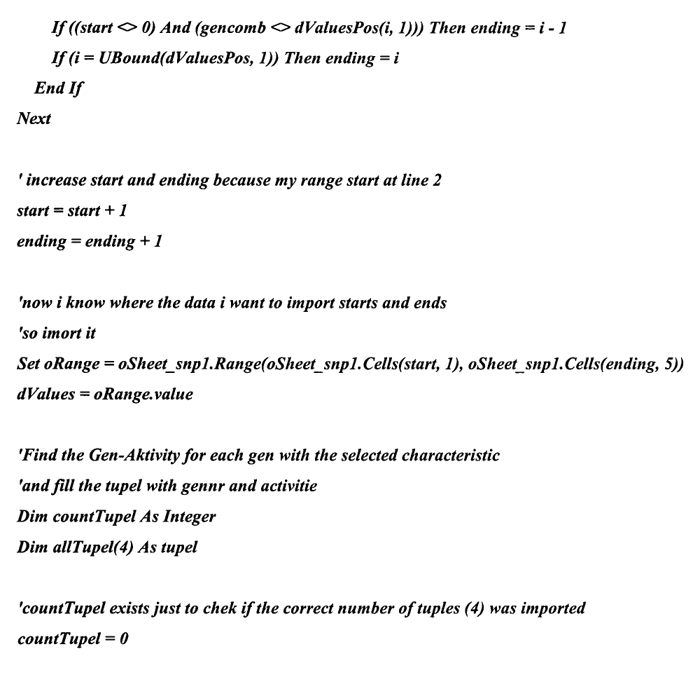

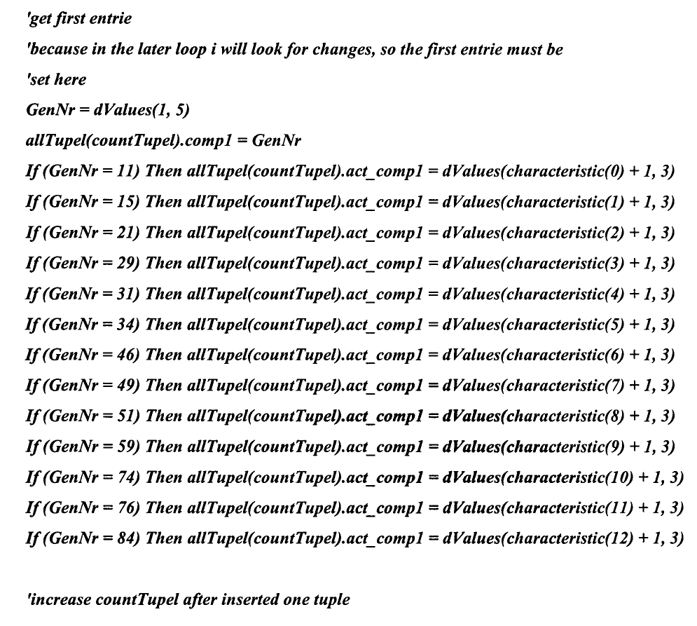

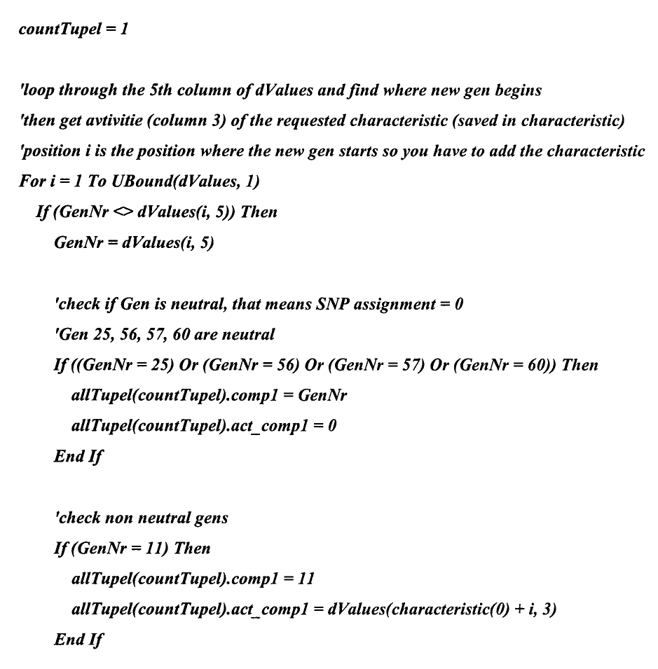

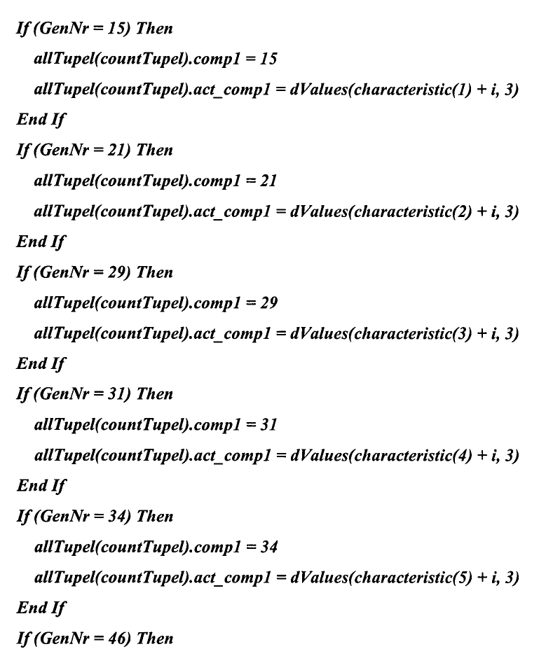

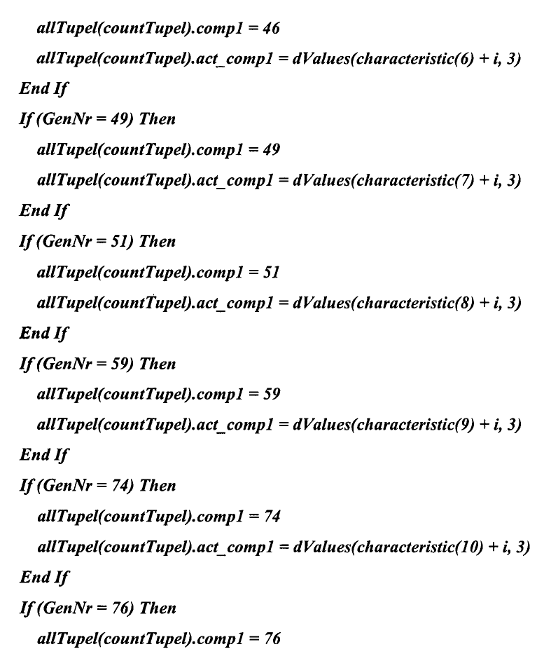

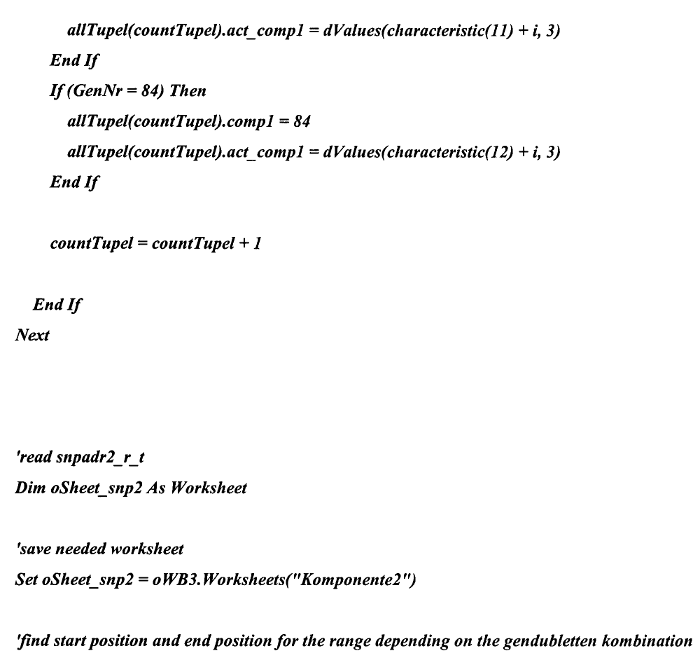

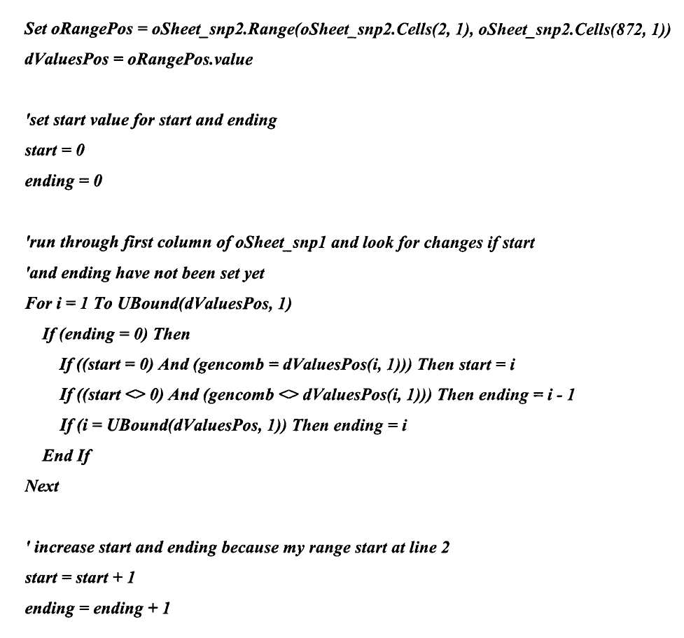

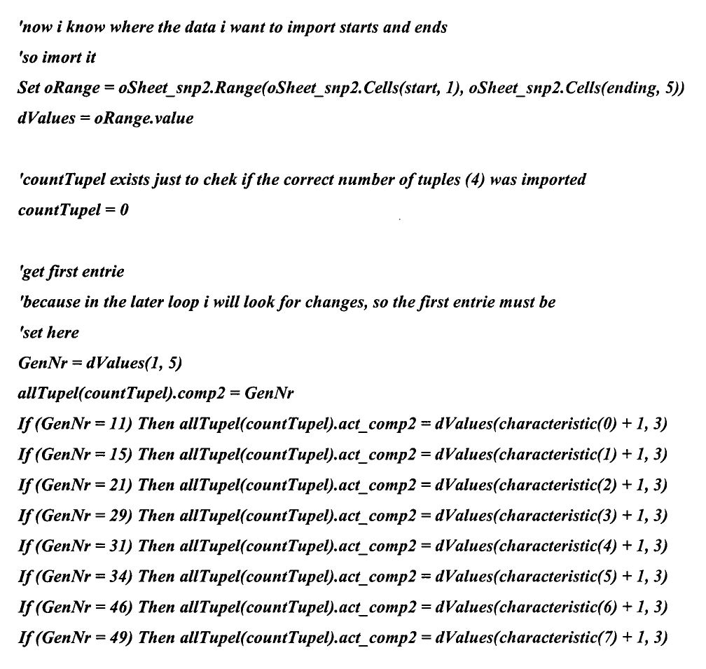

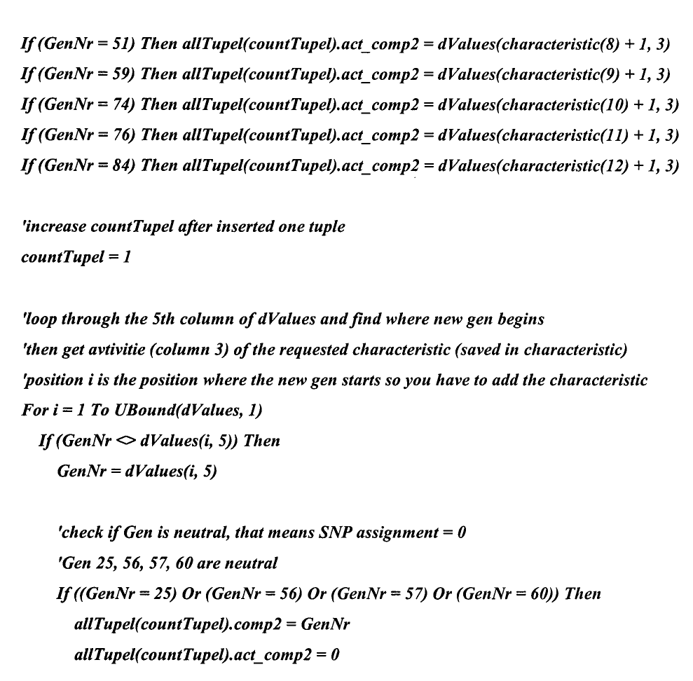

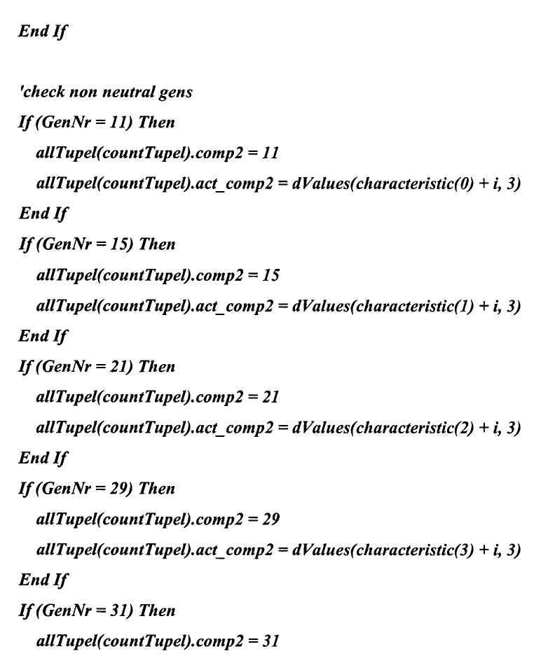

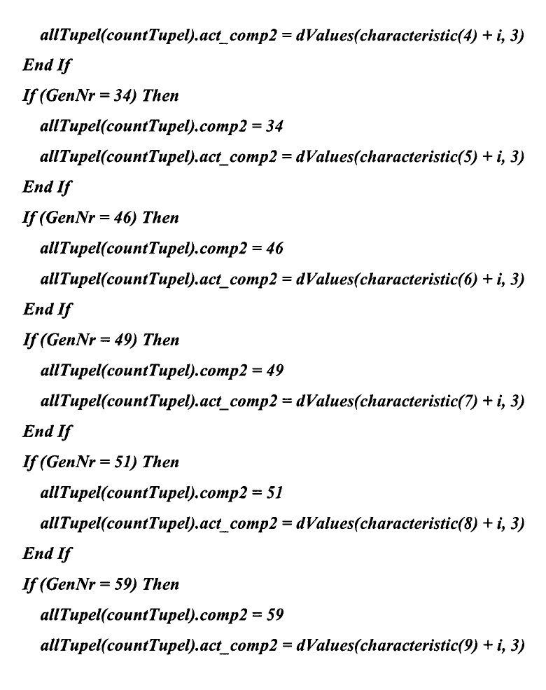

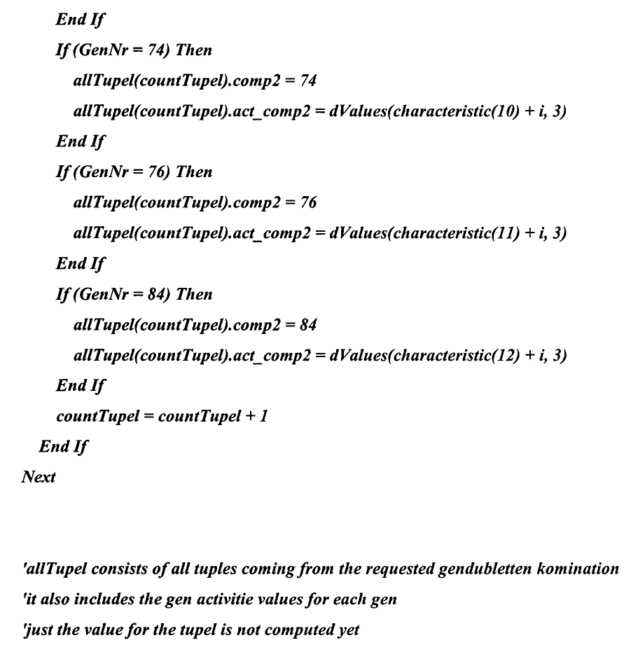

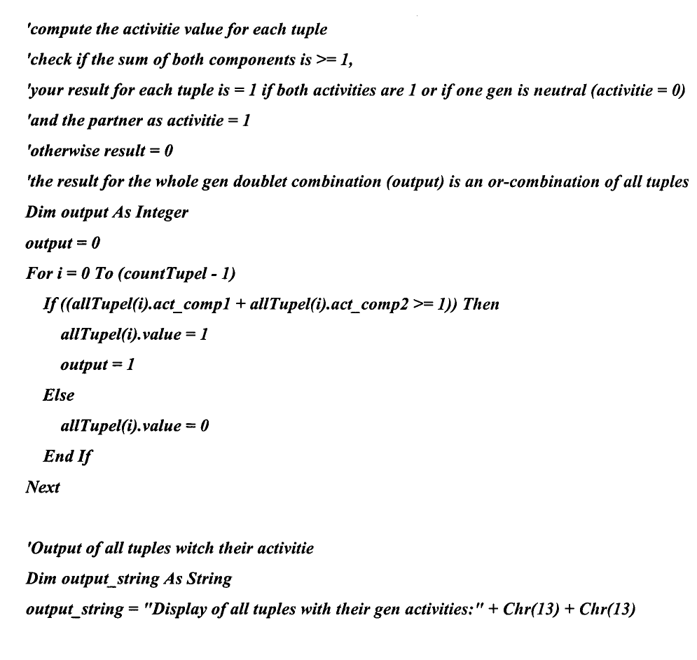

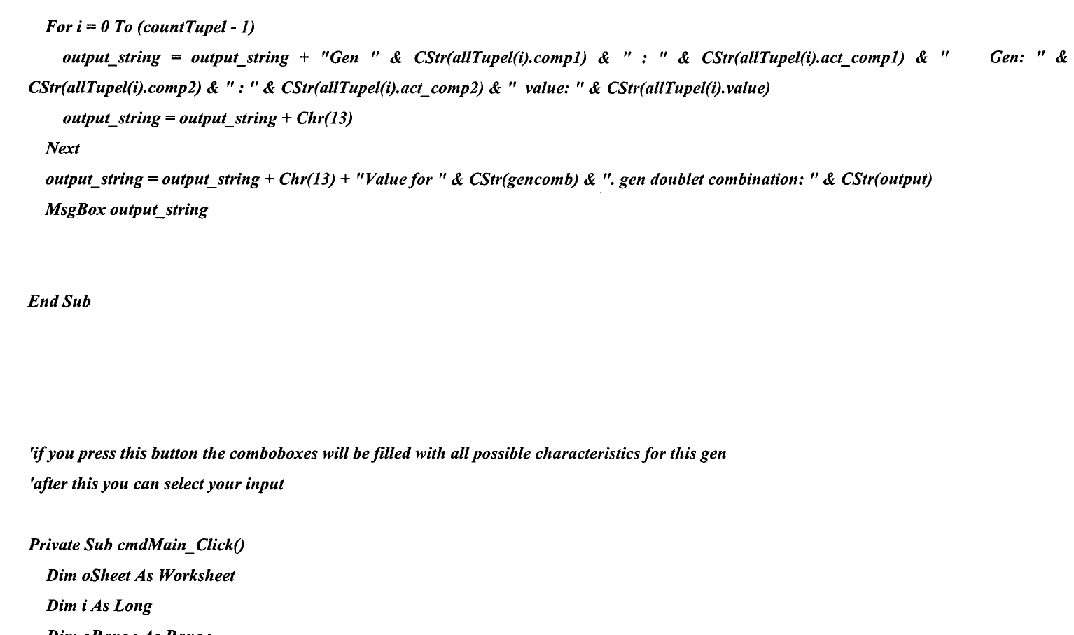

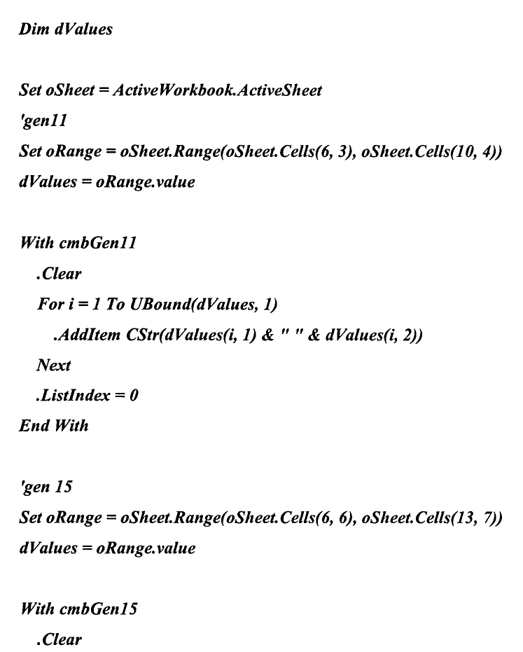

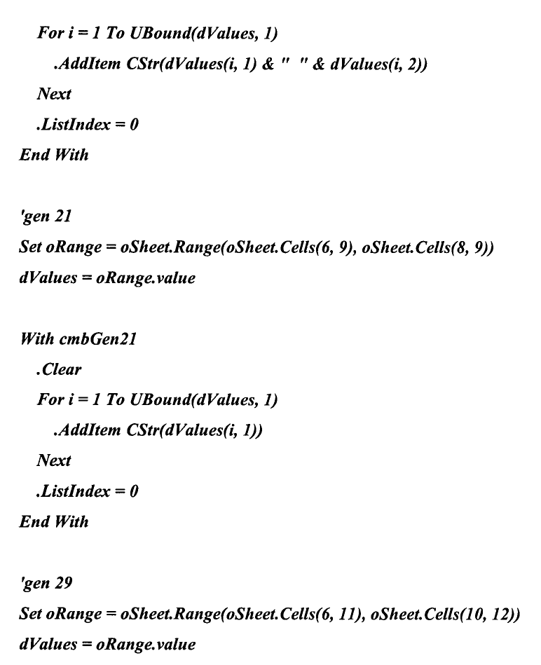

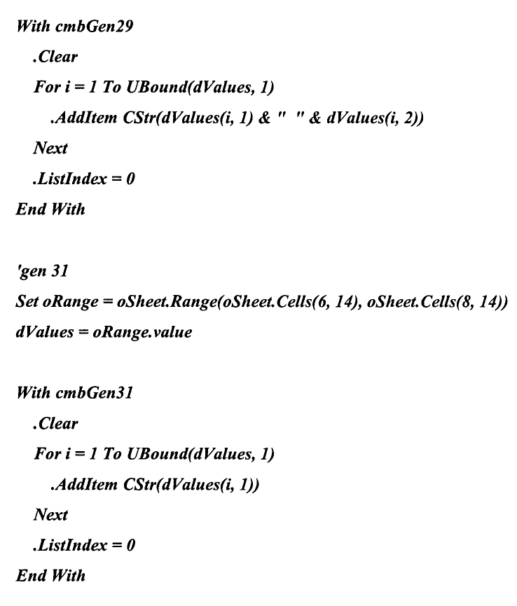

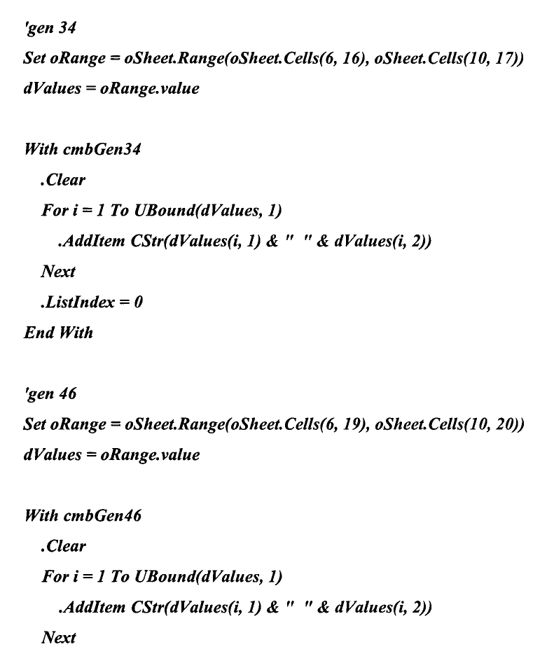

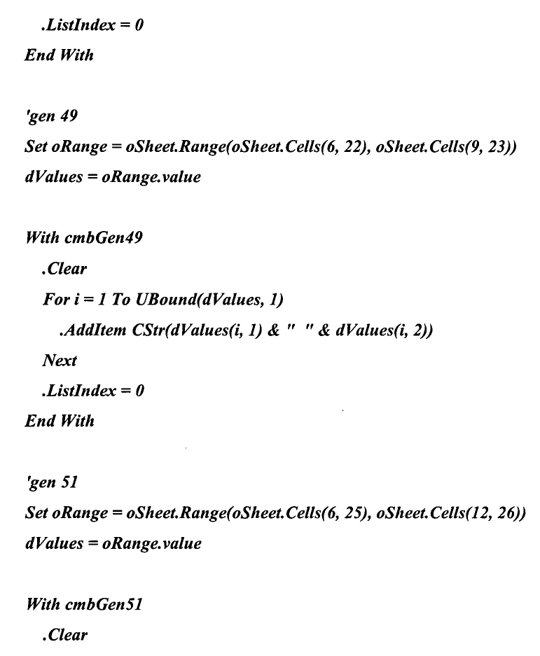

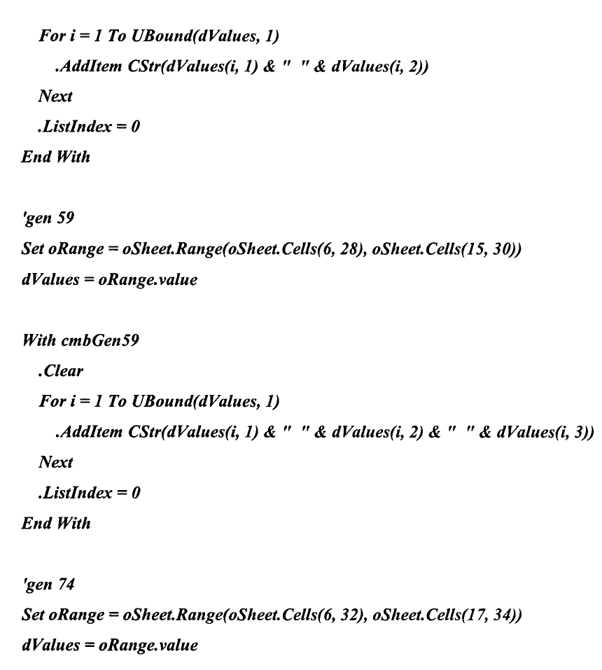

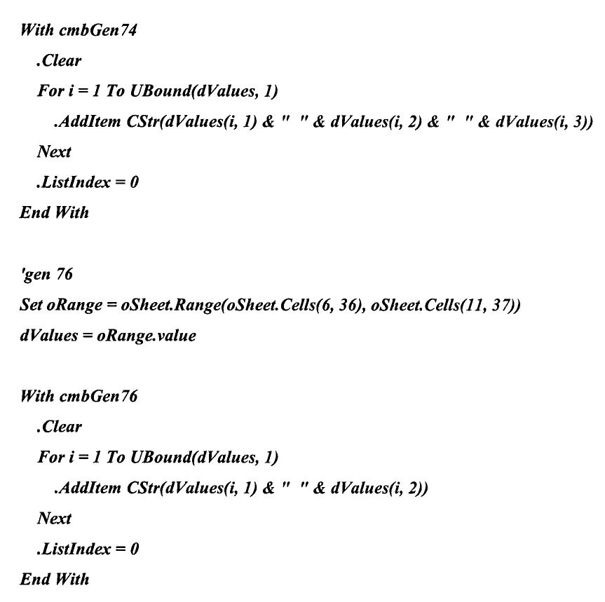

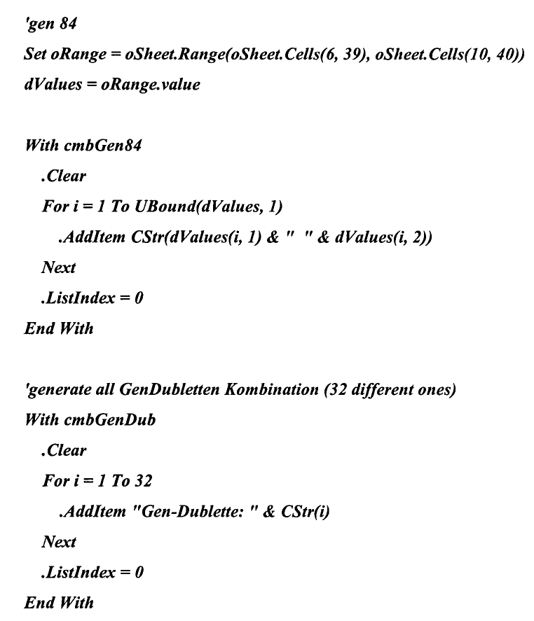

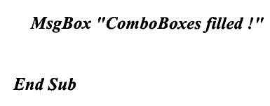

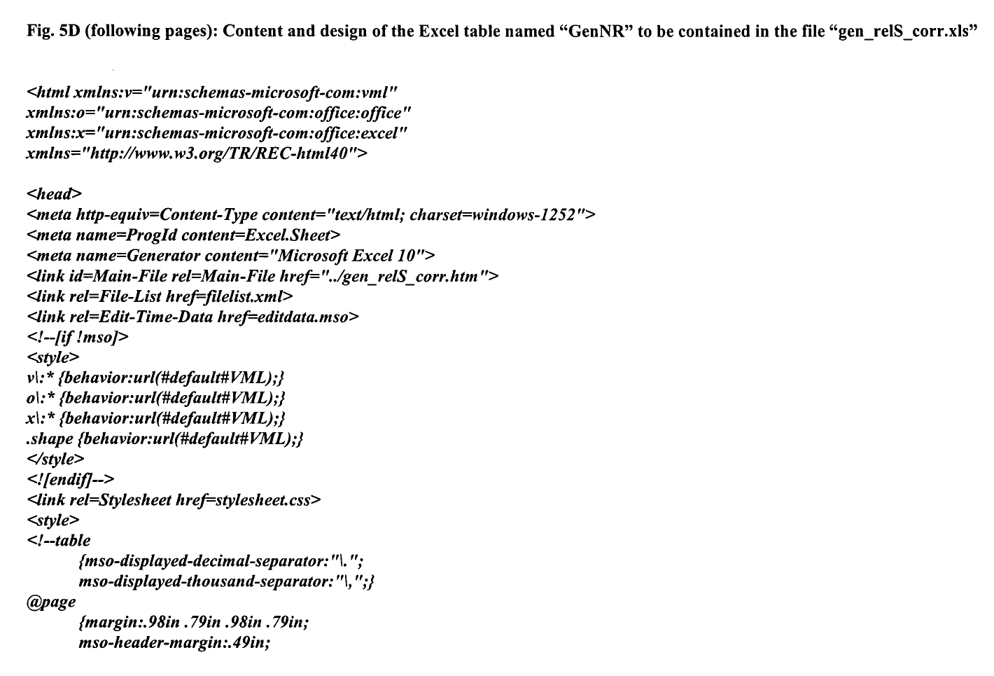

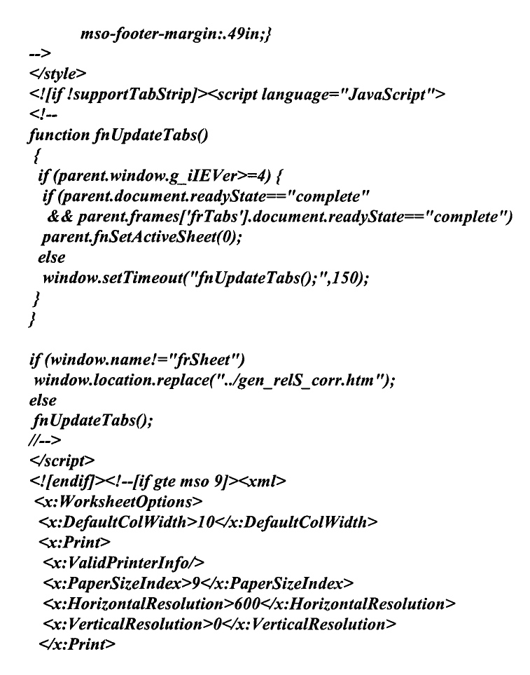

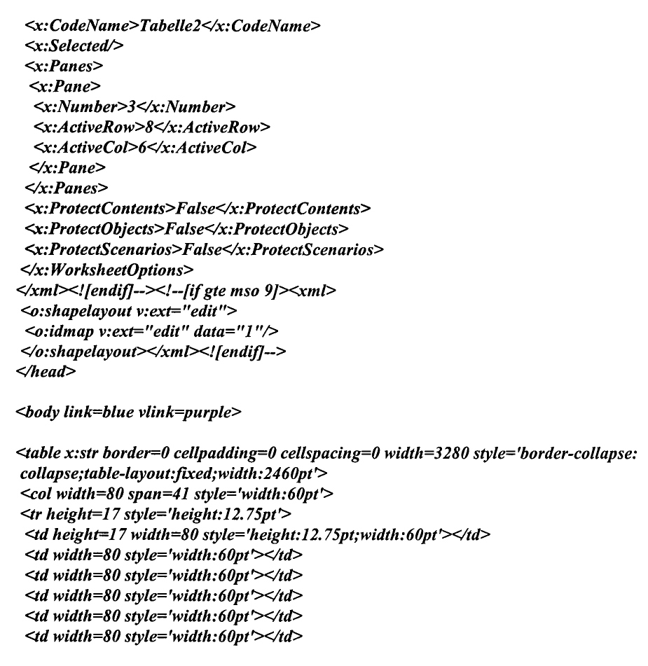

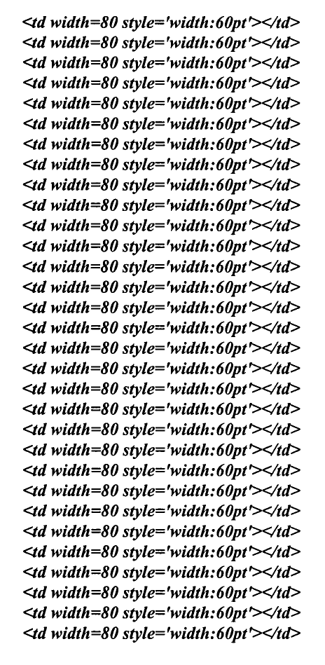

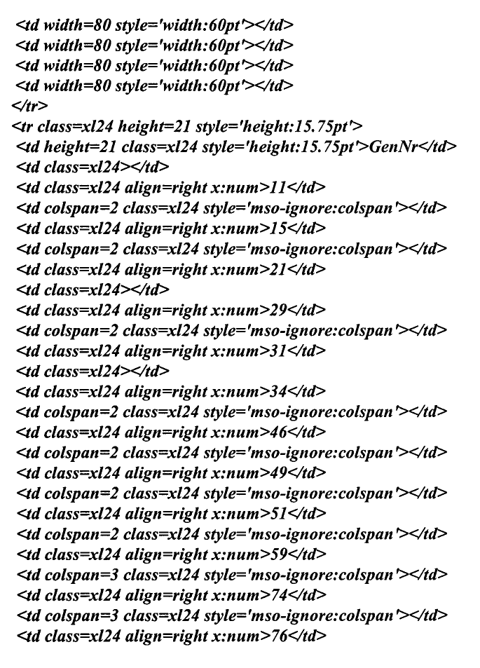

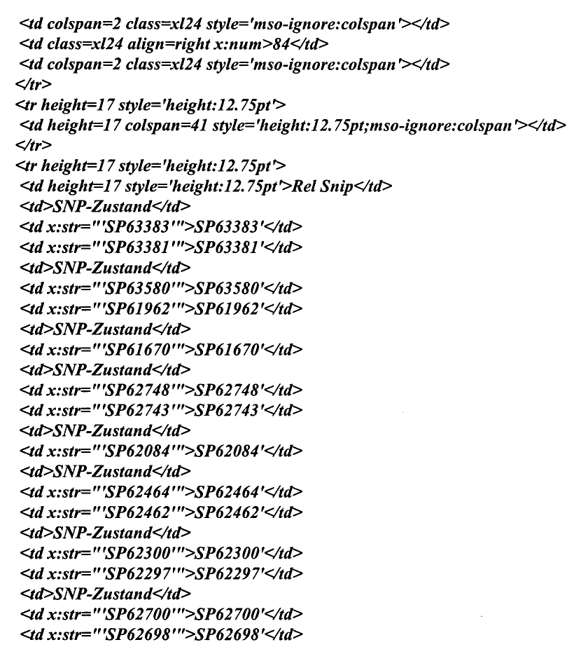

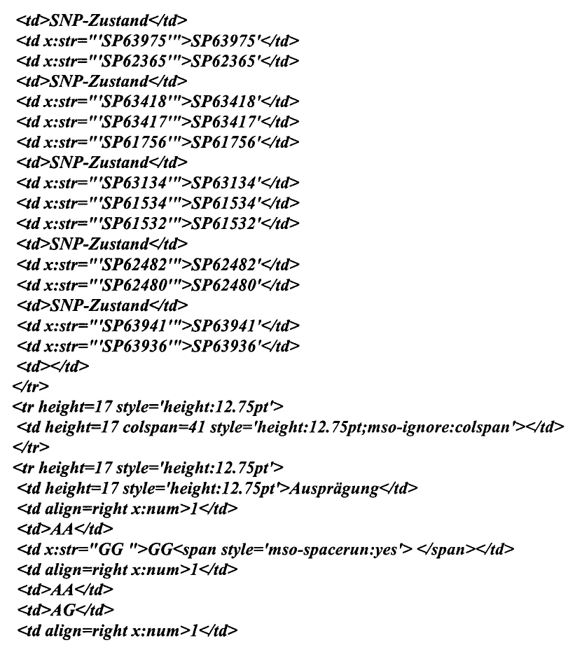

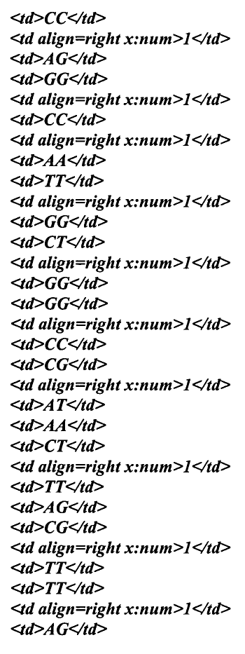

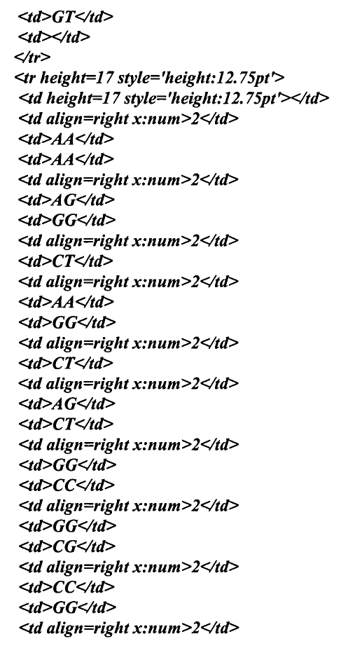

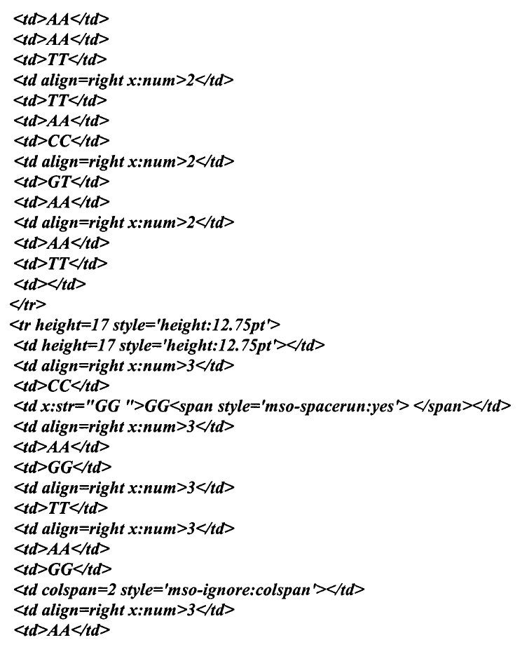

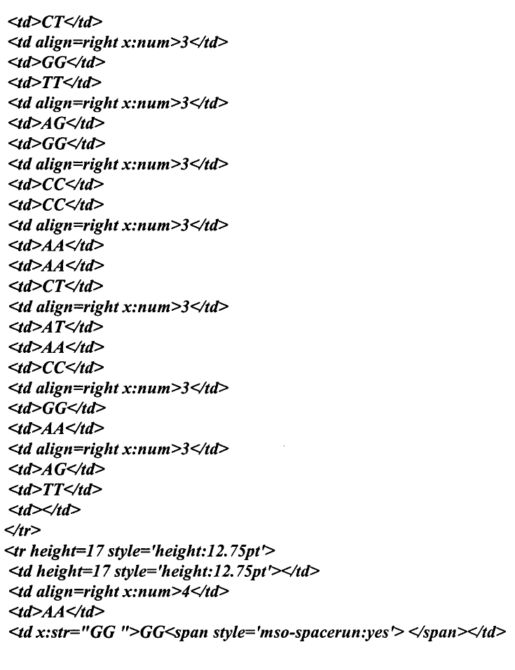

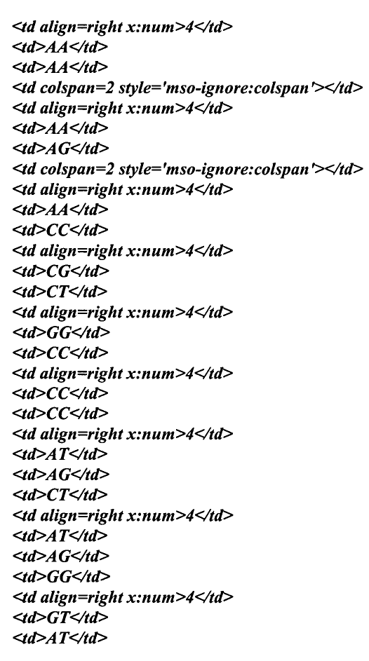

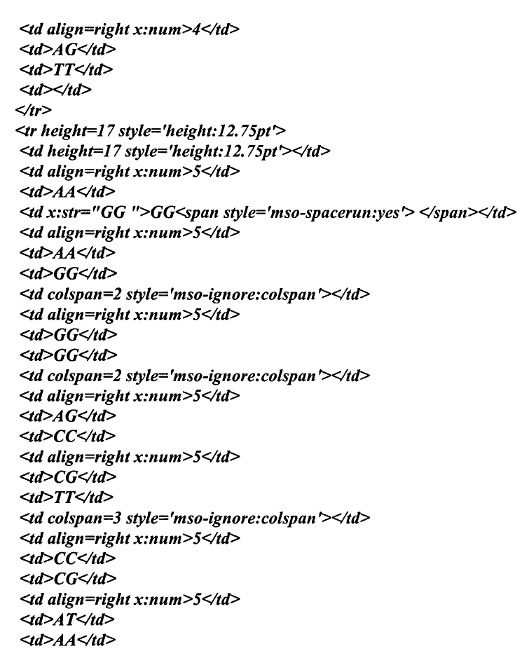

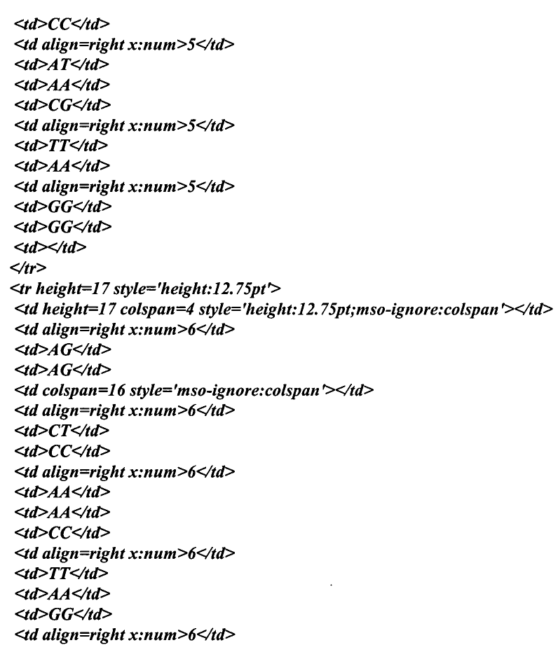

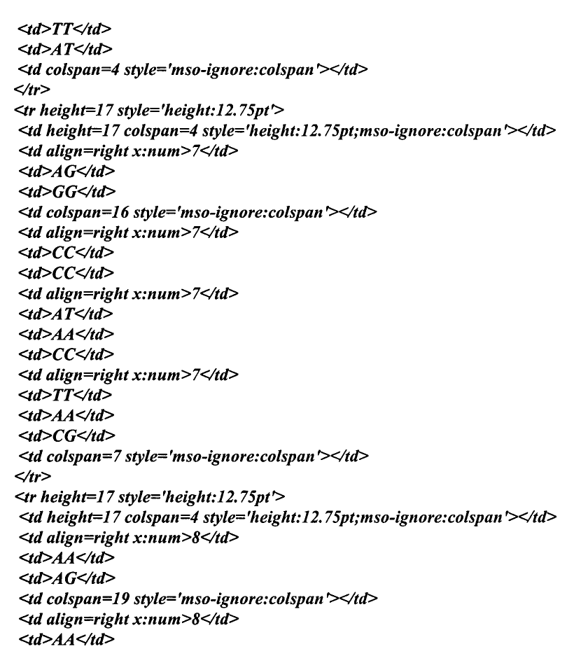

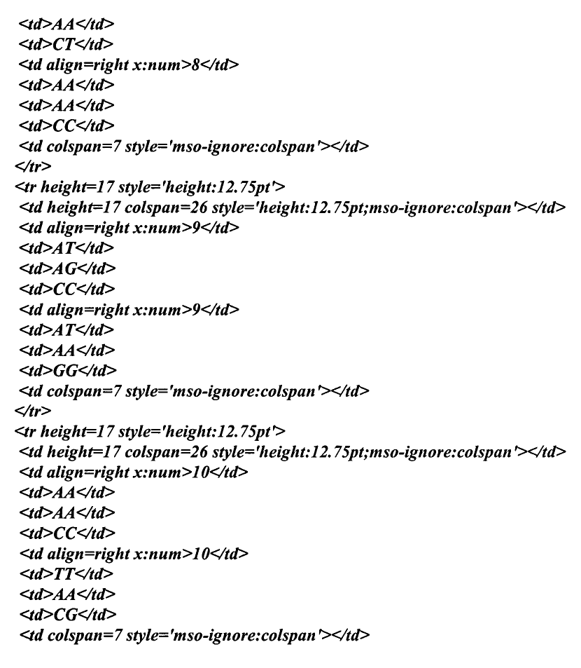

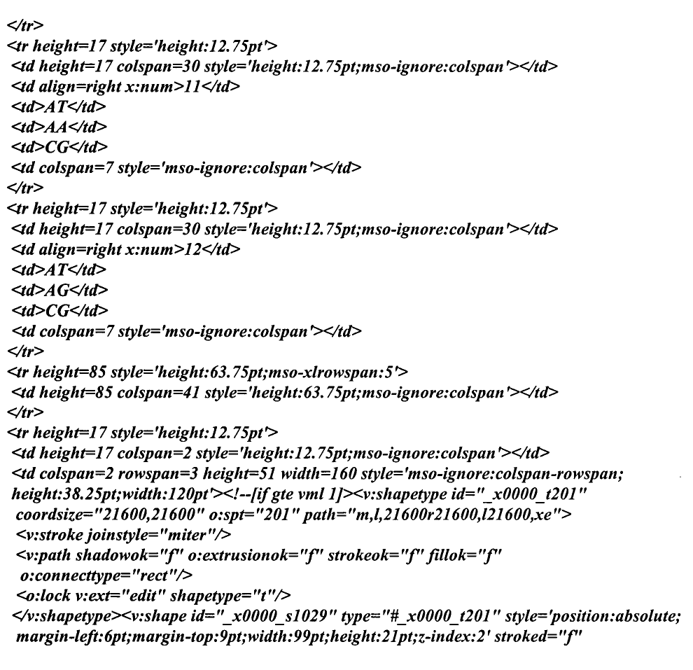

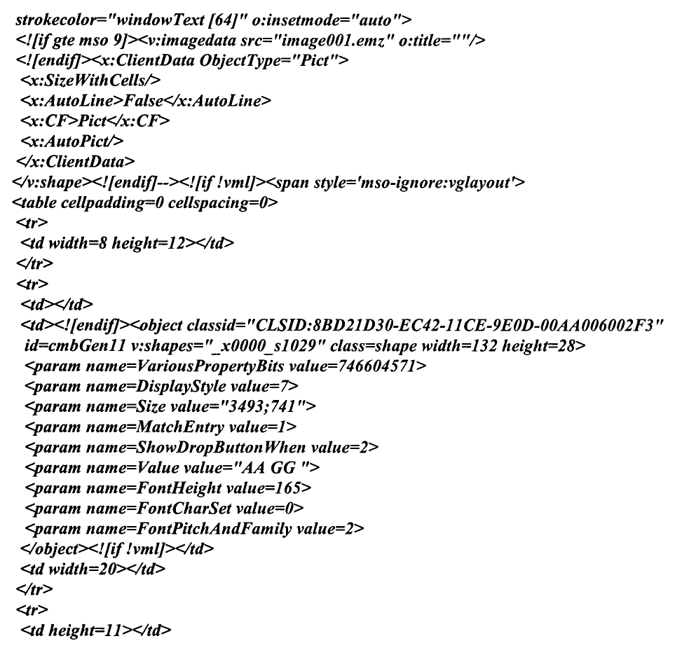

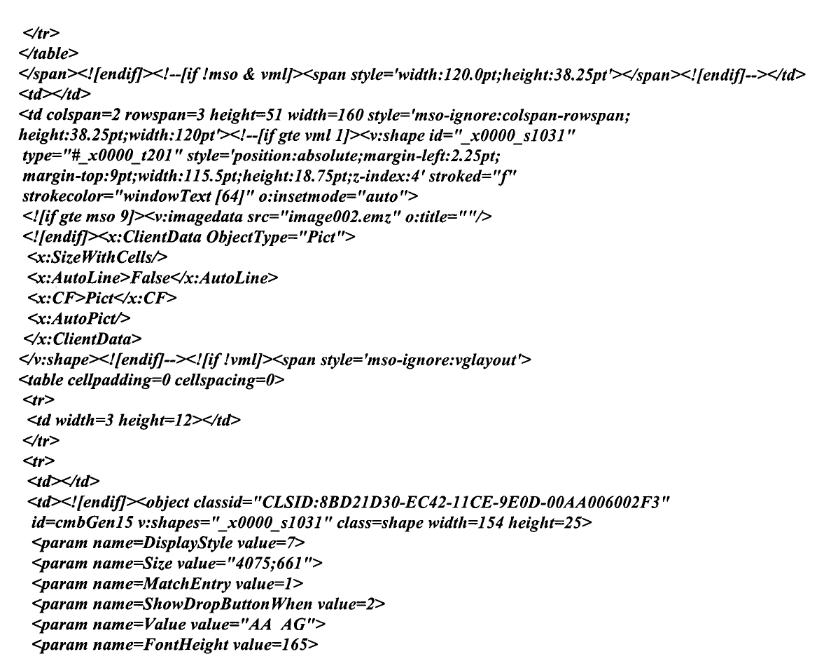

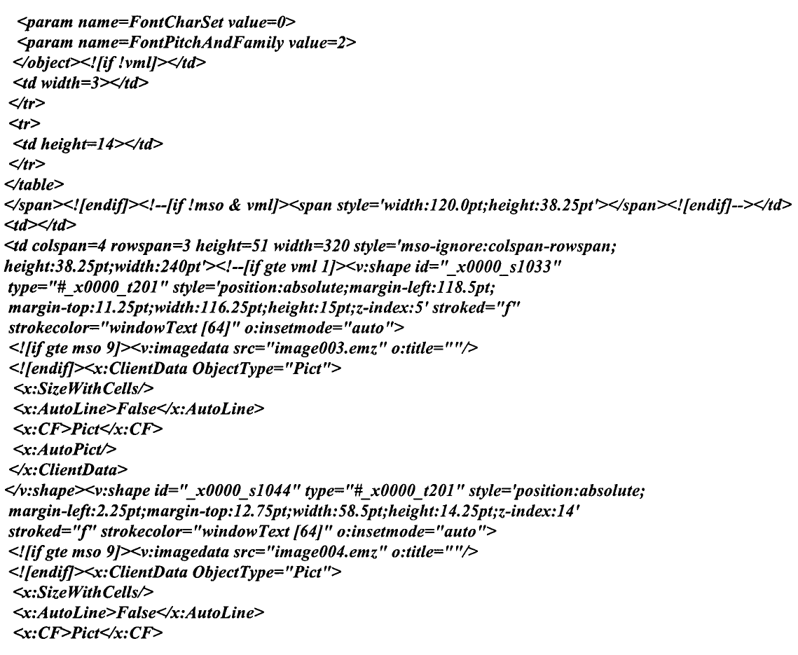

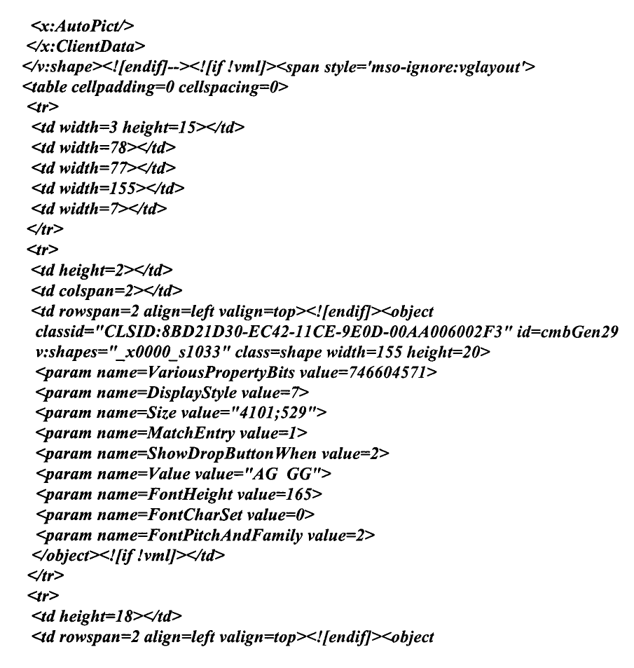

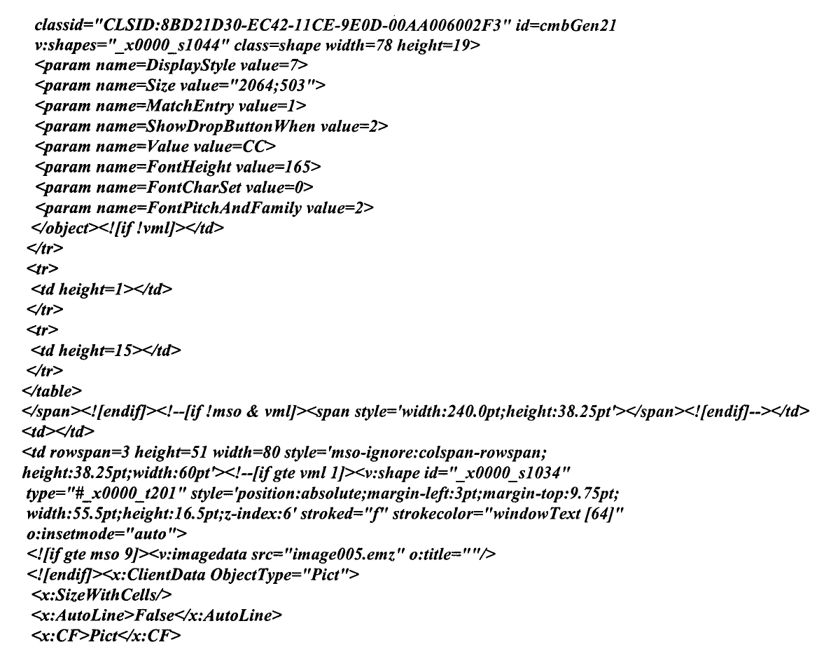

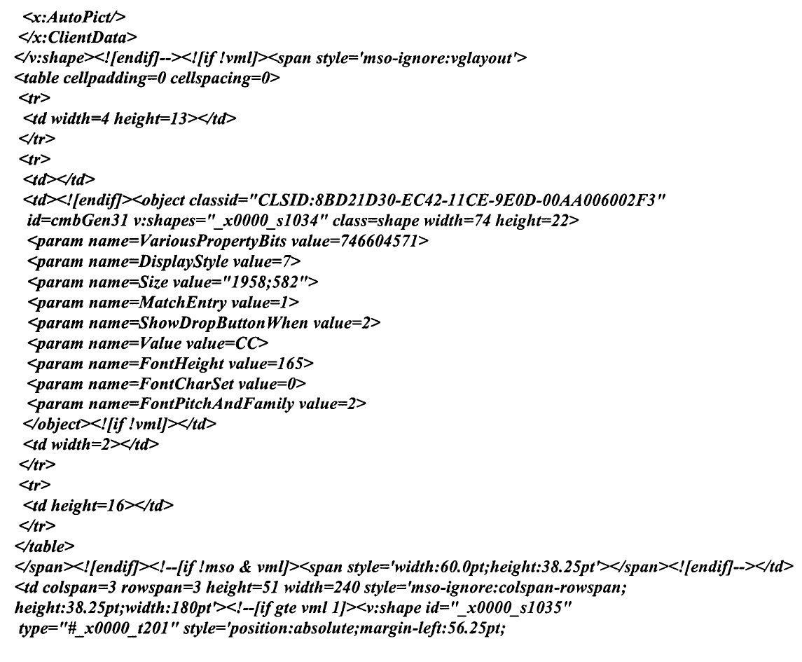

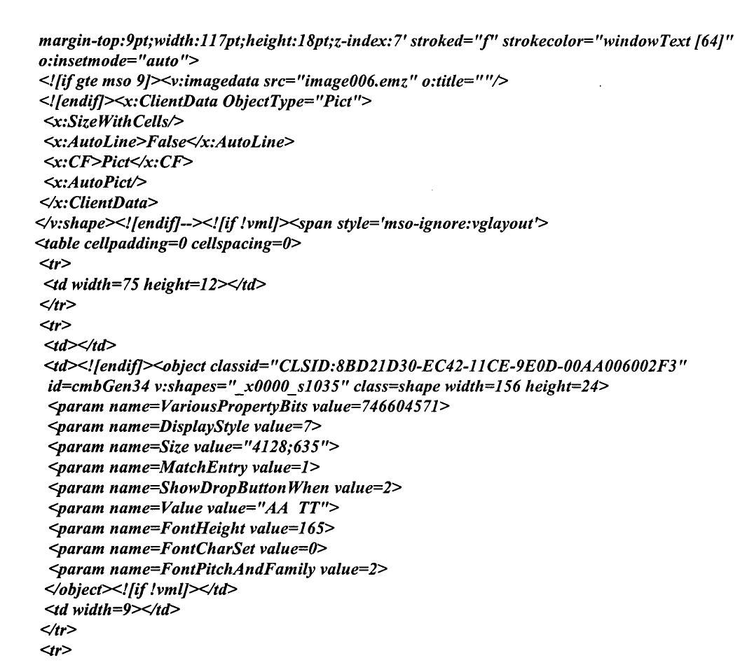

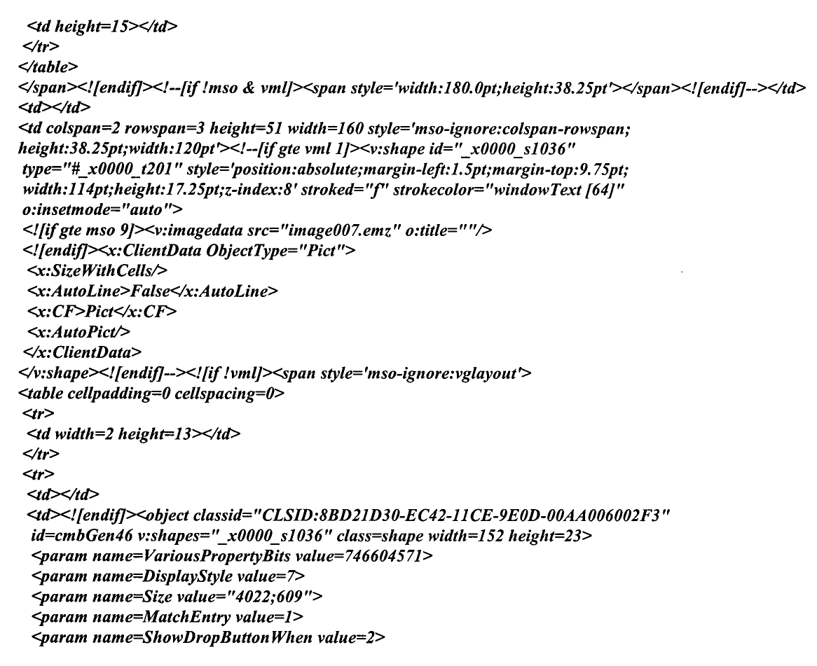

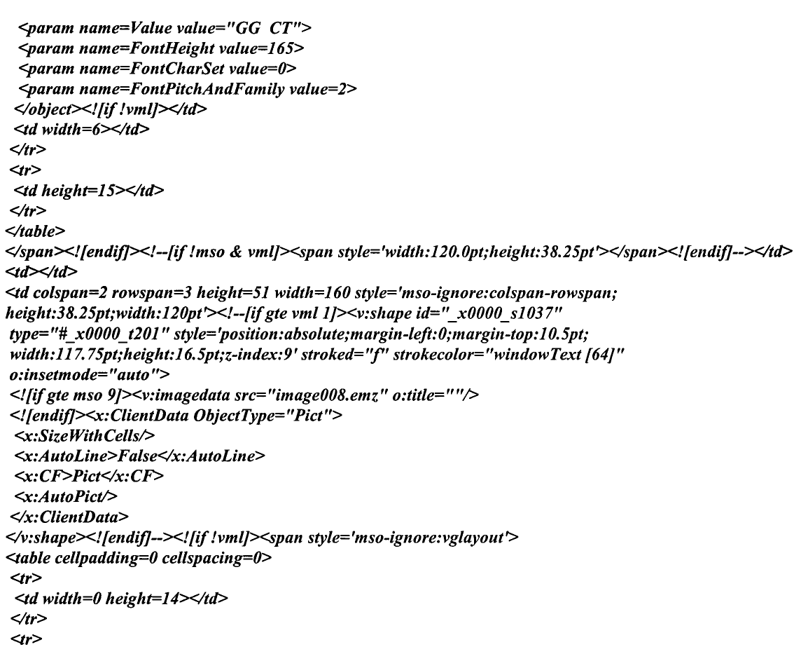

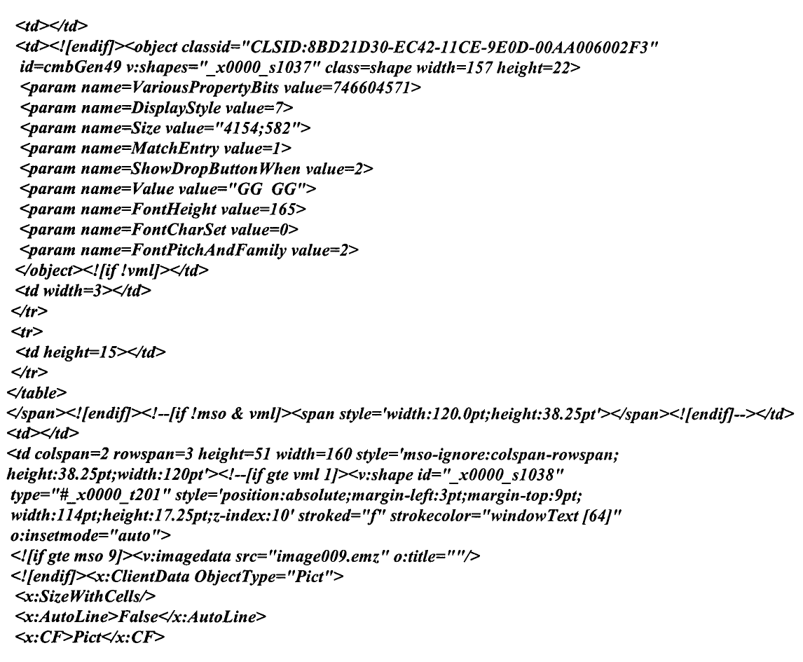

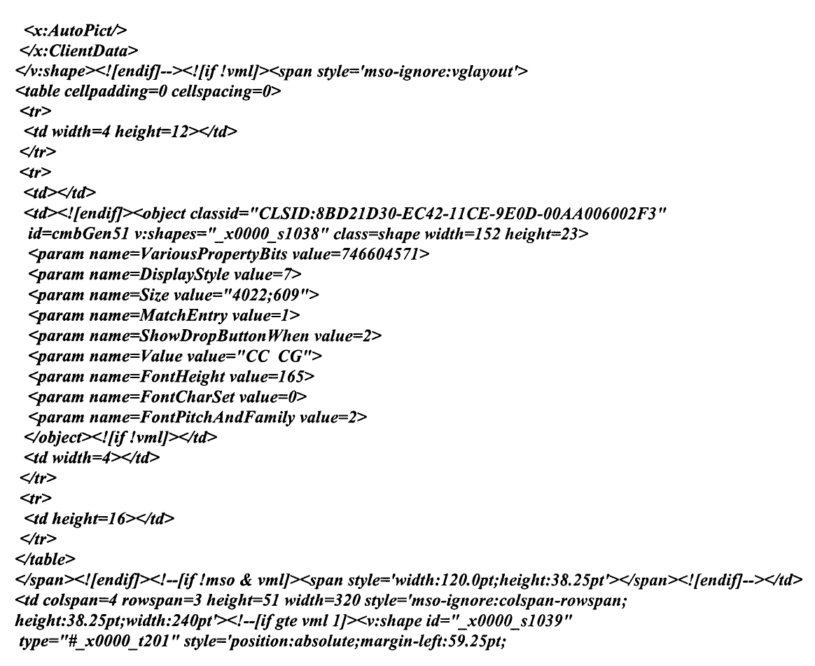

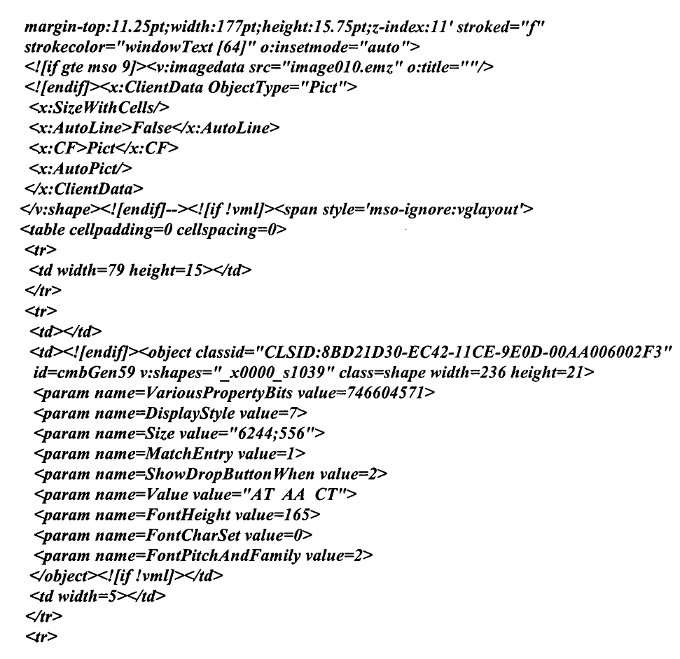

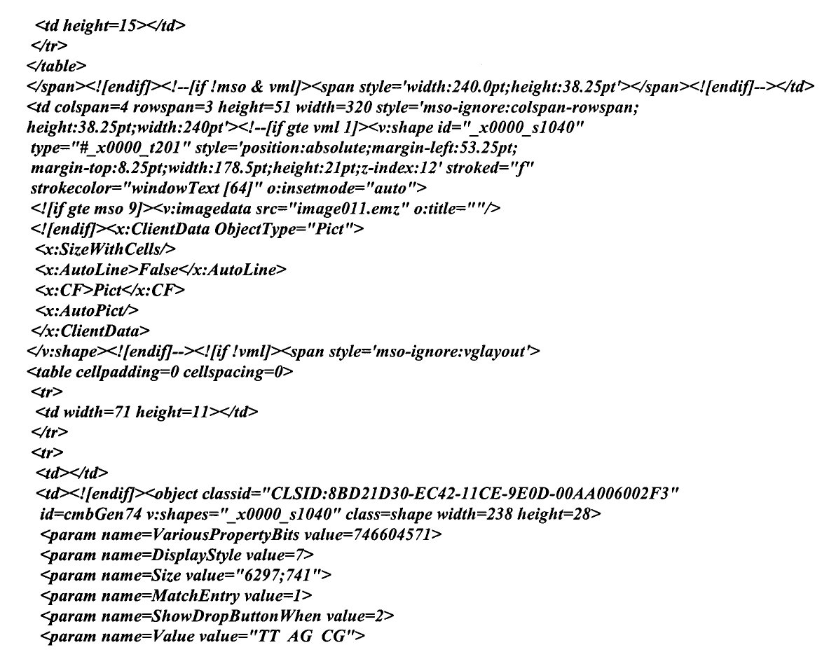

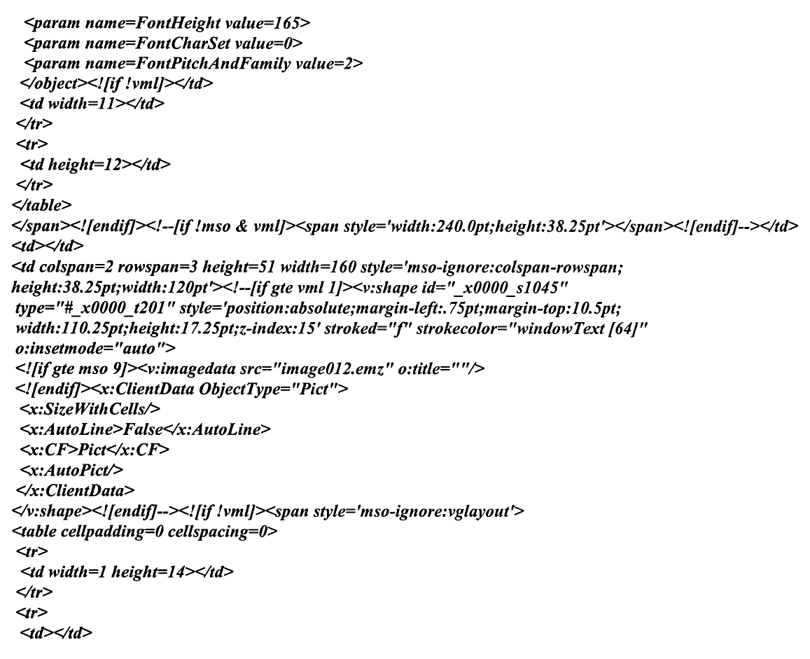

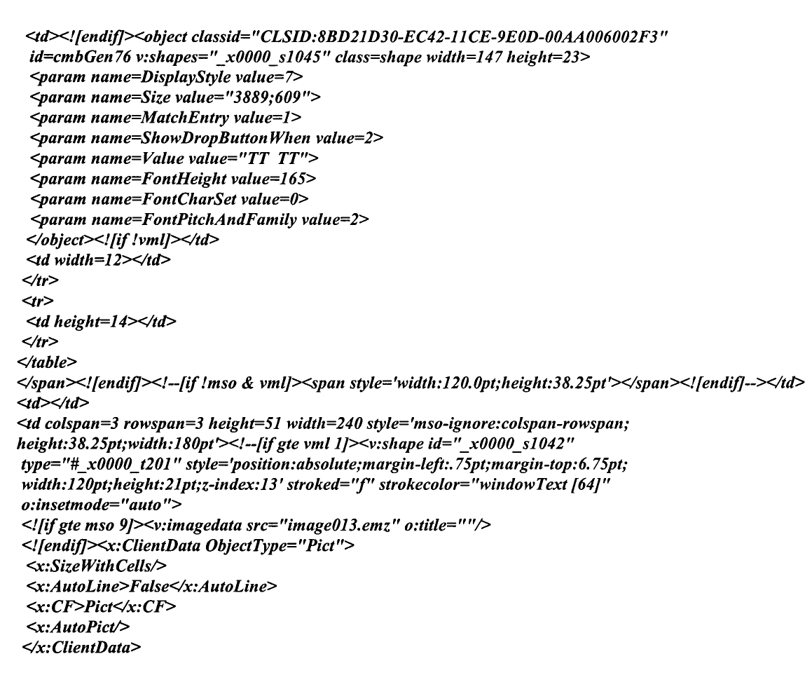

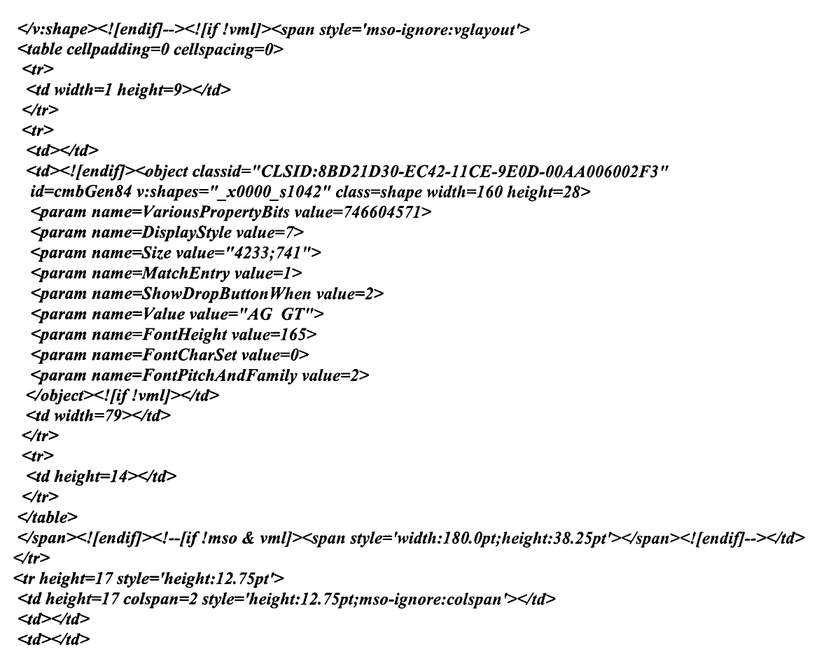

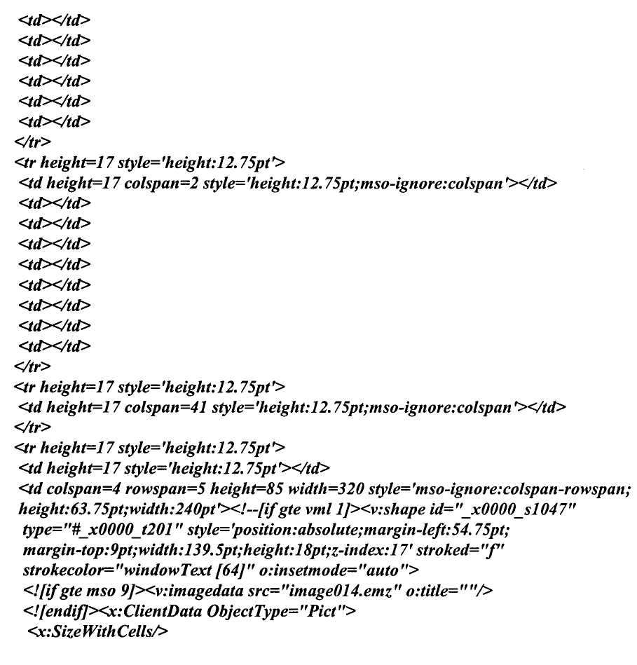

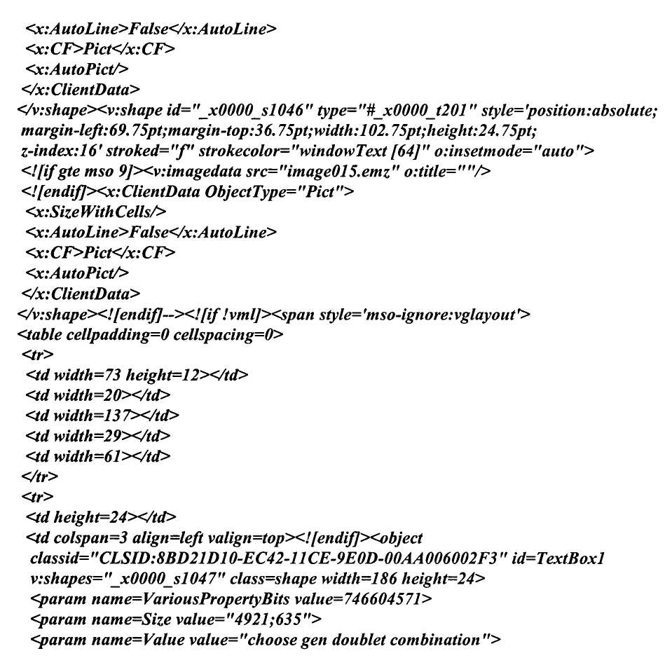

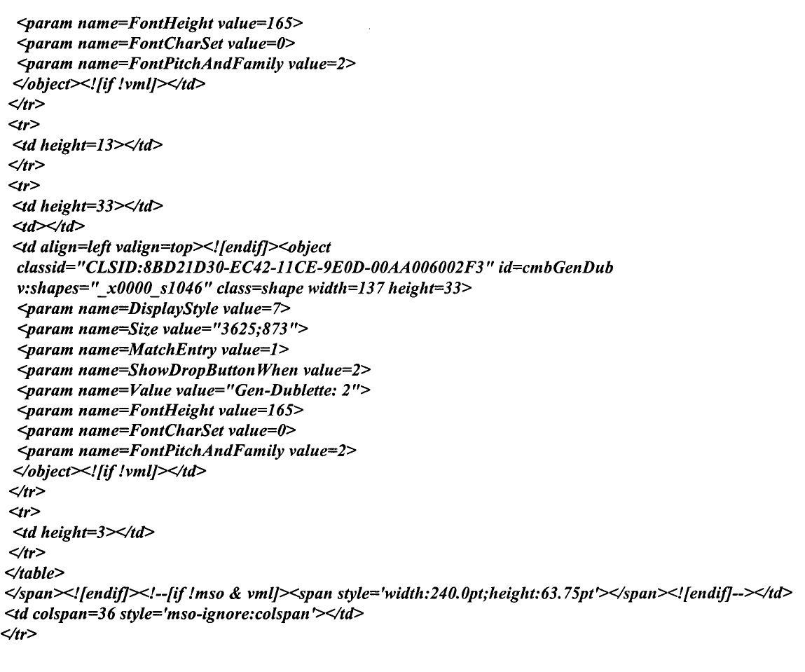

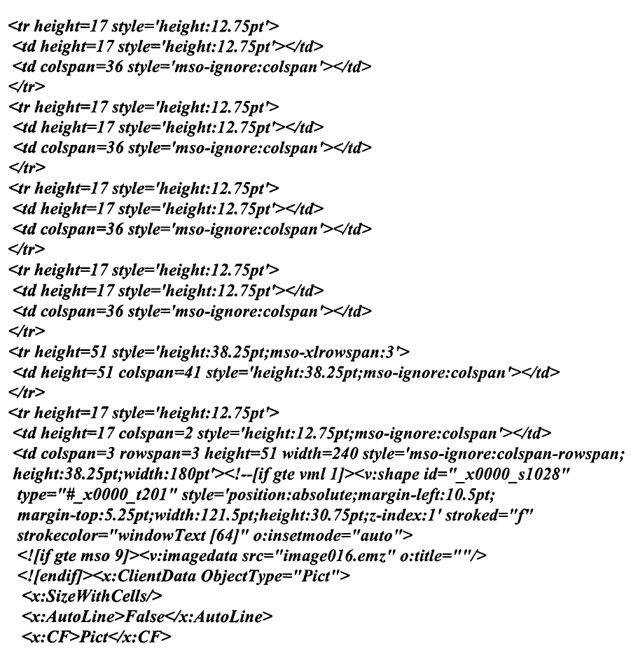

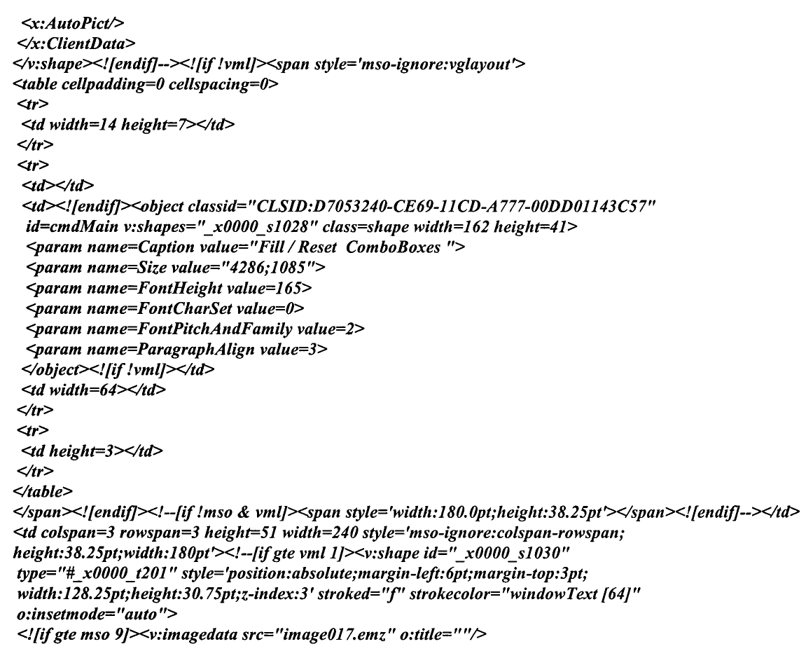

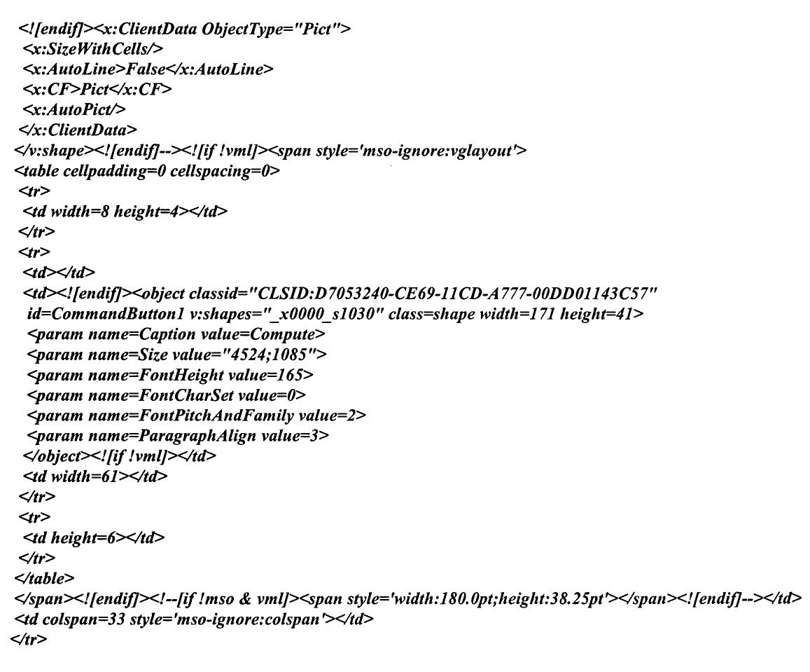

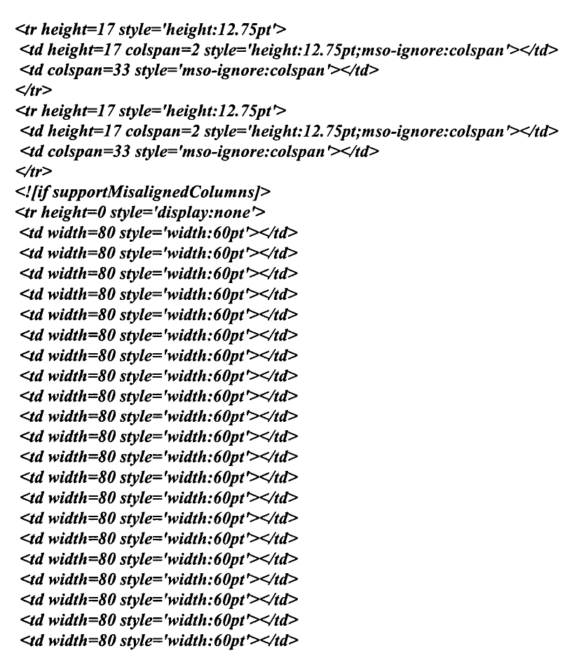

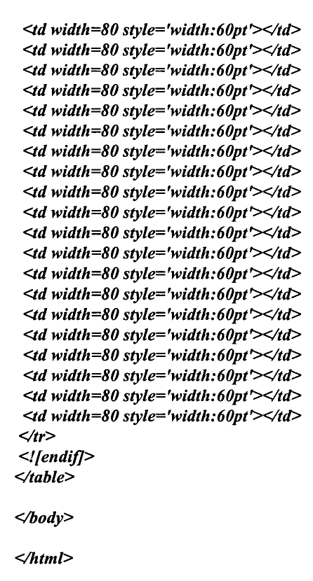

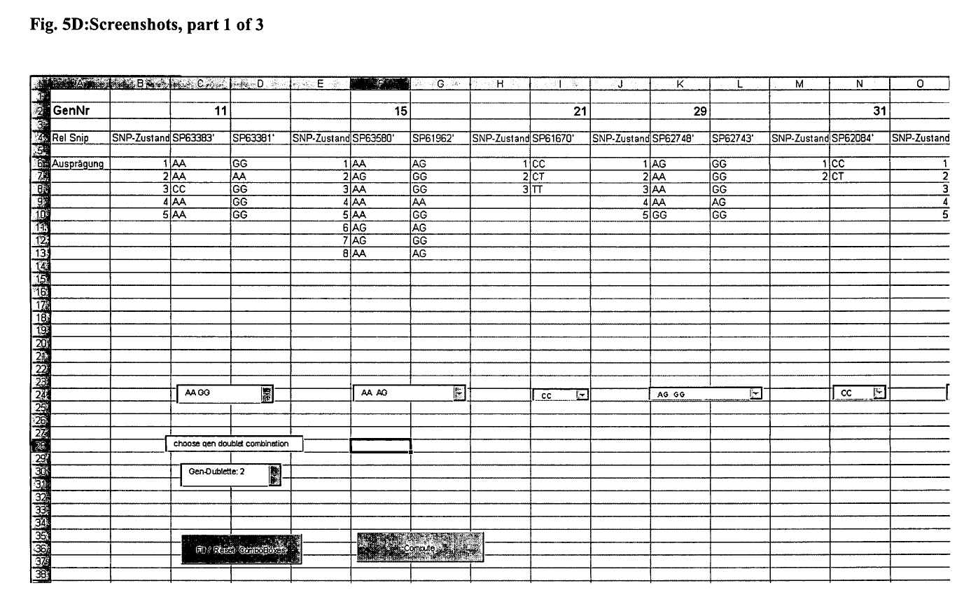

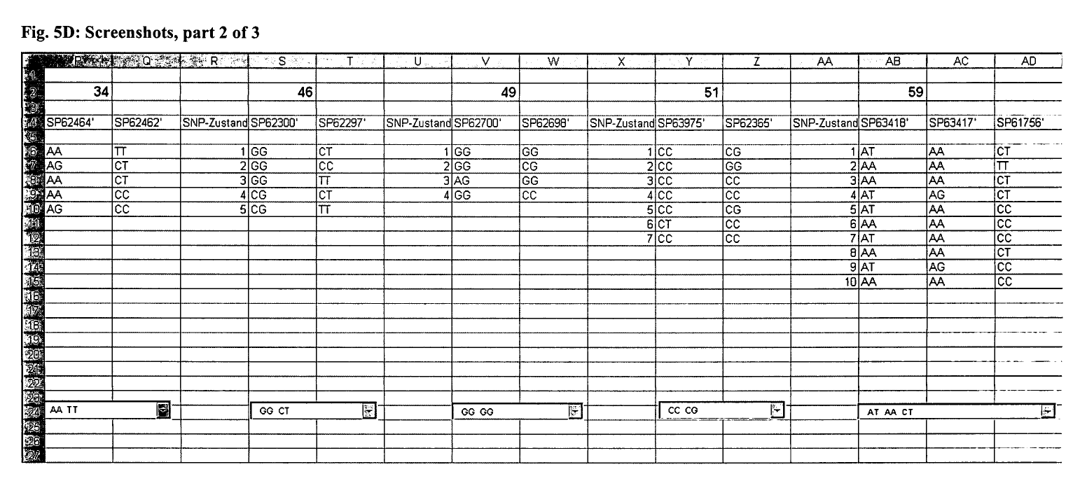

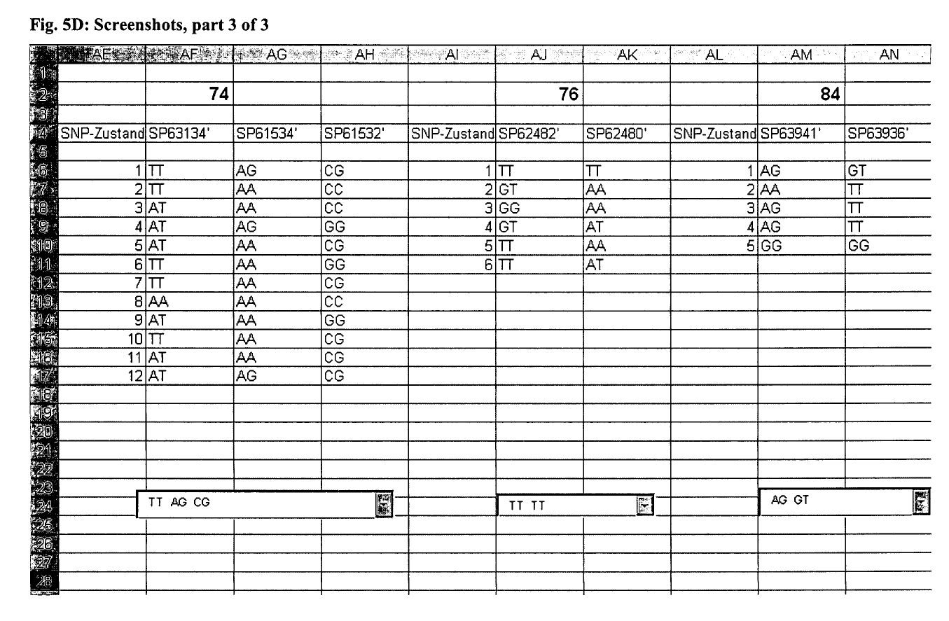

To identify the individual risk for statin-induced adverse drug reactions the following step have to be taken (all measurements are performed BEFORE onset of statin therapy):
1. Measurement of the following parameters in patient blood: Creatine kinase serum activity (CK), LDL serum level, HDL serum level, cholesterol serum level (CHOL), alkaline phosphatase serum activity (AP).
2. Determination of the SNPs as disclosed in FIG. 1 and the sequence listing.
3. Follow decision tree as disclosed in FIG. 3: If the patient cannot be assigned to either CASE or CONTROL, continue with step 4.
4. For class prediction of the remaining individuals, a computer program has been written: Use of the program is described in FIG. 4, the program itself and necessary auxiliary tables are disclosed in FIG. 5A-D (the program was implemented as a Visual Basic Script with Microsoft Excel 2002, Microsoft Corporation, Redmont, Wash., USA). Using this tool, all remaining individuals can be classified into either CASE or CONTROL.

### Sequences:

The sequence section contains all SADR SNPs and adjacent genomic sequences. The position of the polymorphisms that were used for the association studies (‘StatinSNP’) is indicated. Sometimes additional variations are found in the surrounding genomic sequence, that are marked by it's respective IUPAC code. Although those surrounding SNPs were not explicitly analyzed, they likely exhibit a similar association to a phenotype as the StatinSNP (due to linkage disequilibrium, Reich D. E. et al. Nature 411, 199-204, 2001). The SNPs of the invention are listed in FIG. 1 and the sequence listing.

1. Method for predicting drug response in a patient comprising the steps of
(i) classification of said patient to one of several classes of patients using clinical parameters of said patient,
(ii) predicting drug response of said patient from class specific genomic markers.

2. Method of claim 1, wherein the drug response is an adverse drug reaction.

3. Method of claim 1, wherein said genomic markers are a set of SNPs.

4. Method of claim 1 wherein said drug response is adverse drug reaction in statin therapy.

5. Method of claim 1, wherein said clinical parameters are selected from the group consisting of
(i) gender
(ii) creatine kinase serum activity
(iii) LDL serum level
(iv) HDL serum level
(v) cholesterol serum level
(vi) alkaline phosphatase serum activity.

6. Method of claim 1, wherein said drug response is adverse drug reaction in statin therapy and said class specific genomic markers is selected from the group consisting of SEQ ID NO:1-SEQ ID NO:35.

7. Method of claim 5 wherein said adverse drug reactions are myopathies and/or rhabdomyelosis and/or elevated creatine kinase levels.

8. Method of claim 6, comprising the steps of
i) determining the creatine kinase serum activity of a patient, wherein a patient having a creatine kinase serum activity of >80 is defined as being prone to statin adverse drug reactions; and wherein
ii) for the remaining patients the LDL serum level, HDL serum level, cholesterin serum level and/or alkaline phosphatase serum level is determined, wherein a patient showing
a) an LDL level of <=171 and having the SNPs as defined by SEQ ID NO: 31-33, and/or
b) an HDL level of <=59 and having the SNPs as defined by SEQ ID NO: 34-35, and/or
c) an cholesterol serum level of <=266 and having the SNPs as defined by SEQ ID NO: 31-33, and/or
d) an alkaline phosphatase serum activity of >=103 and having the SNPs as defined by SEQ ID NO: 9-11, and/or
e) an alkaline phosphatase serum activity of >=103 and having the SNPs as defined by SEQ ID NO: 6,
is defined as being prone to statin adverse drug reactions; and wherein
iii) remaining patients are screened for the presence of
SNPs as defined by SEQ ID NO: 1-35, wherein a patient showing the SNPs as defined by SEQ ID NO: 1-35 is defined as being prone to statin adverse drug reactions.

9. Method of claim 8, comprising the steps of
i) determining the creatine kinase serum activity of a patient, wherein a patient having a creatine kinase serum activity of >70 is defined as being prone to statin adverse drug reactions; and wherein
ii) for the remaining patients the LDL serum level, HDL serum level, cholesterin serum level and/or alkaline phosphatase serum level is determined, wherein a patient showing
a) an LDL level of <=190 and having the SNPs as defined by SEQ ID NO: 31-33, and/or
b) an HDL level of <=70 and having the SNPs as defined by SEQ ID NO: 34-35, and/or
c) an cholesterol serum level of <=290 and having the SNPs as defined by SEQ ID NO: 31-33, and/or
d) an alkaline phosphatase serum activity of >=90 and having the SNPs as defined by SEQ ID NO: 9-11, and/or
e) an alkaline phosphatase serum activity of >=90 and having the SNPs as defined by SEQ ID NO: 6,
is defined as being prone to statin adverse drug reactions; and wherein
iii) remaining patients are screened for the presence of
SNPs as defined by SEQ ID NO: 1-35, wherein a patient showing the SNPs as defined by SEQ ID NO: 1-35 is defined as being prone to statin adverse drug reactions.

10. Method of selecting a drug for a patient having hypercholisterinaemia, wherein statin drug response is predicted, and statin therapy or an alternative therapy is selected based on the outcome of the prediction, wherein the method of claim 8 is used for statin drug response prediction, and patient still remaining after step iii) of claim 8 are given statin therapy and patients defined as being prone to statin drug response should be assigned by the treating physician to an alternative therapy.

11. Kit, suitable for performing a method according to claim 1.

12. Method according to claim 8, wherein the single nucleotide polymorphisms as defined by SEQ ID NOs: 1-35 are detected on nucleotide basis.

13. Method according to claim 8, wherein the polymorphisms as defined by SEQ ID NOs: 1-35 are defined on polypeptide or protein basis.

14. Method according to claim 12, wherein at least one polymorphism-specific antibody specific for a polymorphism as defined by SEQ ID NOs: 1-35 is used.

15. Polymorphism-specific antibody, characterised in that the antibody is specific for a polymorphism selected from the group of polymorphisms as defined by SEQ ID NOs: 1-35.
